(19) United States
(12) Patent Application Publication
(10) Pub. No.: US 2007/0299113 A1
(43) Pub. Date: Dec. 27, 2007

(54) Metabotropic glutamate receptor modulators

(75) Inventors: Ivars Kalvinsh, Salaspils (LV); Larisa Kaulina, Riga (LV); Valerjans Kauss, Riga (LV); Ronalds Zemribo, Jurmala (LV); Wojciech Danysz, Nidderau (DE); Markus Henrich, Munzenberg (DE); Christopher Graham Raphael Parsons, Nidderau (DE); Tanja Weil, Frankfurt am Main (DE); Elina Erdmane, Riga (LV)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: MERZ PHARMA GMBH & CO. KGAA, FRANKFURT AM MAIN (DE)

(21) Appl. No.: 11/821,492

(22) Filed: Jun. 22, 2007

Related U.S. Application Data
(60) Provisional application No. 60/816,171, filed on Jun. 23, 2006.

(51) Int. Cl. A61K 31/4439; A61K 31/43; C07D 498/04

(52) U.S. Cl. 514/338; 514/366; 546/270.1; 548/154

(57) ABSTRACT

The invention relates to imidazothiazole derivatives as well as their pharmaceutically acceptable salts. The invention further relates to a process for the preparation of such compounds. The compounds of the invention are group I mGluR modulators and are therefore useful for the control and prevention of various disorders, including acute and/or chronic neurological disorders.

### FIELD OF THE INVENTION

The present invention is concerned with novel metabotropic glutamate receptor (mGluR) modulators, methods for their synthesis and the treatment and/or prevention of various diseases and disorders, including neurological disorders, by administration of such substances.

### BACKGROUND OF THE INVENTION

Neuronal stimuli are transmitted by the central nervous system (CNS) through the interaction of a neurotransmitter released by a neuron, which neurotransmitter has a specific effect on a neuroreceptor of another neuron.

L-glutamic acid is considered to be the major excitatory neurotransmitter in the mammalian CNS, consequently playing a critical role in a large number of physiological processes. Glutamate-dependent stimulus receptors are divided into two main groups. The first group comprises ligand-controlled ion channels whereas the second comprises metabotropic glutamate receptors (mGluR). Metabotropic glutamate receptors are a subfamily of G-protein-coupled receptors (GPCR). There is increasing evidence for a peripheral role of both ionotropic and metabotropic glutamate receptors outside of the CNS e.g., in chronic pain states.

At present, eight different members of these mGluRs are known. On the basis of structural parameters such as sequence homology, the second messenger system utilized by these receptors and their different affinity to low-molecular weight compounds, these eight receptors can be divided into three groups: mGluR1 and mGluR5 belong to Group I which couple to phospholipase C and their activation leads to intracellular calcium-ion mobilization. Both mGluR2 and mGluR3 belong to Group II and mGluR4, mGluR6, mGluR7 and mGluR8 belong to Group III, which couple to adenyl cyclase with their activation causing a reduction in second messenger cAMP and as such a dampening of the neuronal activity.

Group I mGluR modulators have been shown to modulate the effects of the presynaptically released neurotransmitter glutamate via postsynaptic mechanisms. Moreover, as these modulators can be both positive and/or negative Group I mGluR modulators, such modulators may increase or inhibit the effects of these metabotropic receptors. Since a variety of pathophysiological processes and disease states affecting the CNS are thought to be related to abnormal glutamate neurotransmission and Group I mGluRs are shown to be expressed in several areas of the CNS, modulators of these receptors could be therapeutically beneficial in the treatment of CNS diseases.

Therefore, group I mGluR modulators may be administered to provide neuroprotection in acute and chronic pathological conditions such as: AIDS-related dementia, Alzheimer's disease, Creutzfeld-Jakob's syndrome, bovine spongiform encephalopathy (BSE) or other prion related infections, diseases involving mitochondrial dysfunction, diseases involving β-amyloid and/or tauopathy such as Down's syndrome, hepatic encephalopathy, Huntington's disease, motor neuron diseases such as amyotrophic lateral sclerosis (ALS), multiple sclerosis (MS), olivoponto-cerebellar atrophy, post-operative cognitive deficit (POCD), lupus disease, neuronal ceroid lipofuscinosis, neurodegenerative cerebellar ataxias, Parkinson's disease, Parkinson's dementia, mild cognitive impairment, dementia pugilisitca, vascular and frontal lobe dementia, cognitive impairment, eye injuries, eye diseases, eye disorders, glaucoma, retinopathy, macular degeneration, head and brain and spinal cord injuries/trauma, hypoglycaemia, hypoxia (e.g. perinatal), ischaemia (e.g. resulting from cardiac arrest, stroke, bypass operations or transplants), convulsions, epilepsy, myoclonic epilepsy, epileptic convulsions, temporal lobe epilepsy, inner ear insult (e.g. in tinnitus, sound or drug-induced), tinnitus, sound or drug-induced tinnitus, L-dopa-induced and tardive dyskinesias, L-dopa-induced dyskinesia in Parkinson's disease therapy, chorea, athetosis, stereotypy, ballism, Tic disorder, torticollis spasmodicus, blepharospasm, focal and generalized dystonia, nystagmus, hereditary cerebellar taxias, corticobasale degeneration, tremor, and essential tremor.

Other indications in this context include a symptomatological effect on the following conditions: abuse and addiction (e.g., nicotine, alcohol, opiate, cocaine, amphetamine), obesity, anxiety and panic disorders, attention deficit hyperactivity disorder (ADHD), attention deficit syndrome, restless leg syndrome, hyperactivity in children, autism, convulsions/epilepsy, dementia (e.g. in Alzheimer's disease, Korsakoff syndrome, vascular dementia, HIV infections), major depressive disorder or depression (including that resulting from Borna virus infection) and bipolar manic-depressive disorder, drug tolerance (e.g. to opioids), movement disorders, dystonia, dyskinesia (e.g. L-Dopa-induced, tardive dyskinesia or in Huntington's disease), fragile-X syndrome, Huntington's chorea, irritable bowel syndrome (IBS), migraine, multiple sclerosis, muscle spasms, pain, chronic pain, acute pain, inflammatory pain, neuropathic pain, diabetic neuropathic pain (DNP), cancer pain, pain related to rheumatic arthritis, allodynia, hyperalgesia, nociceptive pain, post traumatic stress disorder, schizophrenia (positive, cognitive and negative symptoms), spasticity, Tourette's syndrome, urinary incontinence and vomiting, pruritic conditions, pruritis, sleep disorders, micturition disorders, neuromuscular disorder in the lower urinary tract, gastroesophageal reflux disease (GERD), lower esophageal sphincter (LES) disease, functional gastrointestinal disorders, dyspepsia, regurgitation, respiratory tract infection, bulimia nervosa, chronic laryngitis, asthma (e.g. reflux-related asthma), lung disease, eating disorders, obesity and obesity-related disorders, binge eating disorder, agoraphobia, generalized anxiety disorder, obsessive-compulsive disorder, panic disorder, posttraumatic stress disorder, social phobia, substance-induced anxiety disorder, delusional disorder, schizoaffective disorder, schizophreniform disorder, substance-induced psychotic disorder, delirium, or for cognitive enhancement and/or neuroprotection.

Moreover, Group I mGluR modulators may also be therapeutically beneficial in the treatment of various disorders including migration of tumor cells, invasion of tumor cells, adhesion of tumor cells, toxicity of tumor cells, growth of tumor cells, glioma and other tumours, cancer, oral cancer, squamous cell carcinoma (SCC), oral squamous cell carcinoma (SSC), neoplasia, hyperplasia, dysplasia, cancer, carcinoma, sarcoma, oral cancer, squamous cell carcinoma (SCC), oral squamous cell carcinoma (SCC), lung cancer, lung adenocarcinoma, breast cancer, prostate cancer, gastric cancer, liver cancer, colon cancer, colorectal carcinoma, brain tumor, tumor of a nerve tissue, malignant glioma, astroglioma, neuroglioma, neuroblastoma, glioblastoma, medulloblastoma, cancer of skin cells, melanoma, malignant melanoma, epithelial neoplasm, lymphoma, myeloma, Hodgkin's disease, Burkett's lymphoma, leukemia, and thymoma.

Yet further indications for Group I mGluR modulators include those indications wherein a particular condition does not necessarily exist but wherein a particular physiological parameter may be improved through administration of the instant compounds, for example cognitive enhancement.

Positive modulators may be particularly useful in the treatment of positive and negative symptoms in schizophrenia and cognitive deficits in various forms of dementia and mild cognitive impairment.

Among the Group I mGluR modulators, those that exhibit a modulatory effect on mGluR5 receptors and thus may affect conditions or diseases associated with the function of those mGluR5 receptors are of particular interest. In addition to the utility of mGluR5 modulators in preventing and/or treating the conditions and/or diseases mentioned above, mGluR5 positive modulators or agonists may be particularly useful for activation of mGluR5 receptors and thereby for preventing and/or treating conditions or diseases which are alleviated by a mGluR5 receptor agonist. mGluR5 modulators and especially mGluR5 positive modulators or agonists may be particularly useful for preventing and/or treating addiction, neuropathic pain, L-dopa-induced and tardive dyskinesias, ALS, fragile-X syndrome, Parkinson's disease, anxiety disorders, epilepsy, positive and/or negative symptoms of schizophrenia, cognitive impairment, or for cognitive enhancement and neuroprotection.

Moreover, mGluR modulators may have activity when administered in combination with other substances exhibiting neurological effects via different mechanisms. Simultaneous administration of Group I mGluR modulators and NMDA receptor antagonists has also been shown to provide neuroprotection in animal models (Zieminska et al. Acta Neurobiol. Exp., 2006, 66, 301-309; Zieminska et al. Neurochemistry International, 2003, 43, 481-492; and Zieminska et al. Neurochemistry International, 2006, 48, 491-497). Moreover, with respect to the specific compounds studied, the combined therapy exhibited a greater neuroprotective effect than monotherapy with either an mGluR modulator or an NMDA receptor antagonist.

### THE PRESENT INVENTION

We have determined that certain imidazothiazole derivatives are Group I mGluR modulators. Therefore, these substances may be therapeutically beneficial in the treatment of conditions which involve abnormal glutamate neurotransmission or in which modulation of Group I mGluR receptors results in therapeutic benefit. These substances are preferably administered in the form of a pharmaceutical composition, wherein they are present together with one or more pharmaceutically acceptable diluents, carriers, or excipients.

### OBJECTS OF THE INVENTION

It is an object of the present invention to provide novel pharmaceutical compounds which are Group I mGluR modulators and pharmaceutical compositions thereof. It is a further object of the invention to provide a novel method of treating, eliminating, alleviating, palliating, or ameliorating undesirable disorders, including CNS disorders, which involve abnormal glutamate neurotransmission by employing a compound of the invention or a pharmaceutical composition containing the same. An additional object of the invention is the provision of a process for producing the imidazothiazole derivatives.

An additional object of the invention is to provide a novel composition comprising a Group I mGluR modulator and an NMDA receptor antagonist to provide neuroprotection.

Yet additional objects will become apparent hereinafter, and still further objects will be apparent to one skilled in the art.

### SUMMARY OF THE INVENTION

What we therefore believe to be comprised by our invention may be summarized inter alia in the following words:

Compounds of Formula I

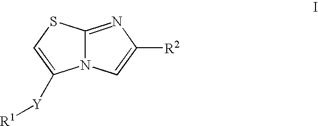

wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroarylC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6a} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;

and optical isomers, polymorphs and pharmaceutically-acceptable acid and base addition salts, hydrates, and solvates thereof;
wherein the term “C₁₋₆alkyl” represents straight or branched chain alkyl groups, examples of such alkyl groups include methyl, ethyl, n-propyl, 2-propyl, n-butyl, 2-butyl, iso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl, and 3-ethylbutyl; the term “C₂₋₆alkenyl” represents straight or branched chain alkenyl groups; the term “cycloC₃₋₁₂alkyl” represents monocyclic, bicyclic or tricyclic alkyl groups including cyclopropyl, cyclobutyl, cyclopentyl, cyclohexyl, bicyclo[2.2.1]heptyl and adamantanyl, wherein the cyclic alkyl group is optionally substituted by one or more substituents, which may be the same or different, selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl” represents phenyl or naphthyl, wherein the phenyl or naphthyl group is optionally substituted by one or more substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-C₁₋₆alkylamino, C₁₋₆alkylcarbonylamino, C₁₋₆alkylsulfonylamino, pyrrolidinyl, piperidinyl, morpholinyl, and piperazinyl or optionally substituted by C₁₋₆alkylenedioxy; the term “heteroaryl” represents an aromatic 5-6 membered ring comprising one to four heteroatoms selected from oxygen, sulfur and nitrogen or a bicyclic ring system having one 5-6 membered ring comprising one to four heteroatoms selected from oxygen, sulfur and nitrogen fused with a benzene ring or a 5-6 membered ring comprising one to four heteroatoms selected from oxygen, sulfur and nitrogen, wherein the heteroaryl is optionally substituted by one or more substituents, which may be the same or different selected independently from halogen, trifluoromethyl, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, pyrrolidinyl, piperidinyl, morpholinyl, pyridyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyl, benzoxazolyl, benzothiazolyl, quinolyl and isoquinolyl; the term “heterocyclyl” represents a saturated or unsaturated non-aromatic 3 to 12 membered ring comprising one to four heteroatoms selected from oxygen, sulfur and nitrogen or a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more substituents selected independently from a halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, pyrrolidinyl, piperidinyl, morpholinyl, pyridyl, and aryl; examples of such heterocyclyl groups include piperidinyl, morpholinyl and piperazinyl; and the term “halogen” represents fluorine, chlorine, bromine and iodine.

Such a compound of Formula I wherein Y represents a single bond, R¹ represents aryl or heteroaryl, and R² represents C₁₋₆alkyl or cycloC₃₋₁₂alkyl.

Such a compound of Formula I wherein R² represents adamantyl.

Such a compound of Formula I wherein
R² represents branched C₁₋₆alkyl, including 2-propyl, 2-butyl, iso-butyl, tert-butyl, 2-pentyl, 3-pentyl, iso-pentyl, 2-methylbutyl, tert-amyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl or 3-ethylbutyl.

Such a compound of Formula I wherein
R² represents branched C₁₋₆alkyl, including 2-propyl, 2-butyl, iso-butyl, tert-butyl, 2-pentyl, 3-pentyl, iso-pentyl, 2-methylbutyl, tert-amyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl or 3-ethylbutyl; Y represents a single bond; and
R¹ represents aryl, including phenyl, optionally substituted by one or more substituents selected independently from C₁₋₆alkyl, including methyl, C₁₋₆alkoxy, including methoxy, halogen, including bromine, C₁₋₆alkoxycarbonyl, including methoxycarbonyl.

Such a compound of Formula I wherein Y represents a single bond, R¹ represents aryl, including phenyl optionally substituted by one or more substituents selected from C₁₋₆alkyl, including methyl, C₁₋₆alkoxy, including methoxy, halogen, including bromine, C₁₋₆alkoxycarbonyl, including methoxycarbonyl, and R² represents Z-R^{6a}, wherein Z represents CR⁷R⁸ and R^{6a} represents aryl or cycloC₃₋₁₂alkyl.

Such a compound of Formula I wherein R⁷ and R⁸, which may be the same or different, each independently represent C₁₋₆alkyl, including methyl, and R^{6a} represents phenyl optionally substituted by one or more substituents selected from C₁₋₆alkyl and halogen (including fluorine), or cycloC₃₋₁₂alkyl (including cyclopentyl and cyclohexyl).

Such a compound of Formula I wherein Y represents a single bond, R¹ represents aryl or heteroaryl, and R² represents C(═O)R^{6b} or C(R⁷)(R⁸)—NR¹⁰R¹¹.

Such a compound of Formula I wherein R^{6b} represents C₁₋₆alkyl, including t-butyl, or cycloC₃₋₁₂alkyl, including cyclohexyl.

Such a compound of Formula I wherein R⁷ and R⁸, which may be the same or different, each independently represent hydrogen or C₁₋₆alkyl, including methyl; and R¹⁰ and R¹¹, together with the nitrogen atom to which they are attached form a monocyclic ring, including piperidine, wherein the ring may be optionally substituted by one or more substituents, which may be the same or different, independently selected from C₁₋₆alkyl, C₁₋₆alkoxy, and halogen.

Moreover, a method for treating or preventing a condition or disease associated with abnormal glutamate neurotransmission or a method for modulating Group I mGluR receptors to achieve therapeutic benefit, or a method for enhancing cognition, such method comprising administering to a living animal, including a human, a therapeutically effective amount of a compound selected from those of Formula I

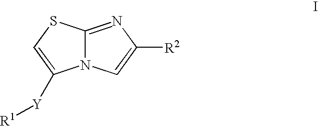

wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroarylC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6a} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;

and optical isomers, polymorphs and pharmaceutically-acceptable acid and base addition salts, hydrates, and solvates thereof;
wherein the term “C₁₋₆alkyl” represents straight or branched chain alkyl groups, examples of such alkyl groups include methyl, ethyl, n-propyl, 2-propyl, n-butyl, 2-butyl, iso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl, and 3-ethylbutyl; the term “C₂₋₆alkenyl” represents straight or branched chain alkenyl groups; the term “cycloC₃₋₁₂alkyl” represents monocyclic, bicyclic or tricyclic alkyl groups including cyclopropyl, cyclobutyl, cyclopentyl, cyclohexyl, bicyclo[2.2.1]heptyl and adamantanyl, wherein the monocyclic, bicyclic or tricyclic alkyl group is optionally substituted by one or more substituents, which may be the same or different, selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl” represents phenyl or naphthyl, wherein the phenyl or naphthyl group is optionally substituted by one or more substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-C₁₋₆alkylamino, C₁₋₆alkylcarbonylamino, C₁₋₆alkylsulfonylamino, pyrrolidinyl, piperidinyl, morpholinyl, and piperazinyl or optionally substituted by C₁₋₆alkylenedioxy; the term “heteroaryl” represents an aromatic 5-6 membered ring comprising one to four heteroatoms selected from oxygen, sulfur and nitrogen or a bicyclic ring system having one 5-6 membered ring comprising one to four heteroatoms selected from oxygen, sulfur and nitrogen fused with a benzene ring or a 5-6 membered ring comprising one to four heteroatoms selected from oxygen, sulfur and nitrogen, wherein the heteroaryl is optionally substituted by one or more substituents, which may be the same or different, selected independently from halogen, trifluoromethyl, C₁₋₆ alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, pyrrolidinyl, piperidinyl, morpholinyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyl, benzoxazolyl, benzothiazolyl, quinolinyl and isoquinolinyl; the term “heterocyclyl” represents a saturated or unsaturated non-aromatic 3 to 12 membered ring comprising one to four heteroatoms selected from oxygen, sulfur and nitrogen or a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more substituents selected independently from a halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, pyrrolidinyl, piperidinyl, morpholinyl, pyridinyl, and aryl; examples of such heterocyclyl groups include piperidinyl, morpholinyl or piperazinyl; and the term “halogen” represents fluorine, chlorine, bromine and iodine.

Such a method wherein the condition associated with abnormal glutamate neurotransmission, or wherein modulation of mGluR receptors results in therapeutic benefit, is selected from: AIDS-related dementia, Alzheimer's disease, Creutzfeld-Jakob's syndrome, bovine spongiform encephalopathy (BSE) or other prion related infections, diseases involving mitochondrial dysfunction, diseases involving β-amyloid and/or tauopathy such as Down's syndrome, hepatic encephalopathy, Huntington's disease, motor neuron diseases such as amyotrophic lateral sclerosis (ALS), multiple sclerosis (MS), olivoponto-cerebellar atrophy, post-operative cognitive deficit (POCD), lupus disease, neuronal ceroid lipofuscinosis, neurodegenerative cerebellar ataxias, Parkinson's disease, Parkinson's dementia, mild cognitive impairment, dementia pugilistica, vascular and frontal lobe dementia, cognitive impairment, eye injuries, eye diseases, eye disorders, glaucoma, retinopathy, macular degeneration, head and brain and spinal cord injuries, head and brain and spinal cord trauma, hypoglycaemia, hypoxia, perinatal hypoxia, ischaemia, ischaemia resulting from cardiac arrest or stroke or bypass operations or transplants, convulsions, epilepsy, myoclonic epilepsy, epileptic convulsions, temporal lobe epilepsy, glioma and other tumours, cancer, oral cancer, squamous cell carcinoma (SCC), oral squamous cell carcinoma (SSC), neoplasia, hyperplasia, dysplasia, cancer, carcinoma, sarcoma, oral cancer, squamous cell carcinoma (SCC), oral squamous cell carcinoma (SCC), lung cancer, lung adenocarcinoma, breast cancer, prostate cancer, gastric cancer, liver cancer, colon cancer, colorectal carcinoma, brain tumor, tumor of a nerve tissue, malignant glioma, astroglioma, neuroglioma, neuroblastoma, glioblastoma, medulloblastoma, cancer of skin cells, melanoma, malignant melanoma, epithelial neoplasm, lymphoma, myeloma, Hodgkin's disease, Burkett's lymphoma, leukemia, thymoma, inner ear insult, inner ear insult in tinnitus, tinnitus, sound or drug-induced inner ear insult, sound or drug-induced tinnitus, L-dopa-induced and tardive dyskinesias, L-dopa-induced dyskinesia in Parkinson's disease therapy, chorea, athetosis, stereotypy, ballism, Tic disorder, torticollis spasmodicus, blepharospasm, focal and generalized dystonia, nystagmus, hereditary cerebellar taxias, corticobasale degeneration, tremor, essential tremor, abuse, addiction, nicotine addiction, nicotine abuse, alcohol addiction, alcohol abuse, opiate addiction, opiate abuse, cocaine addiction, cocaine abuse, amphetamine addiction, amphetamine abuse, obesity, anxiety and panic disorders, attention deficit hyperactivity disorder (ADHD), attention deficit syndrome (ADS), restless leg syndrome, hyperactivity in children, autism, dementia, dementia in Alzheimer's disease, dementia in Korsakoff syndrome, vascular dementia, dementia in HIV infections, major depressive disorder or depression, depression resulting from Borna virus infection, and bipolar manic-depressive disorder, drug tolerance, drug tolerance to opioids, movement disorders, dystonia, dyskinesia, L-Dopa-induced dyskinesia, tardive dyskinesia, dyskinesia in Huntington's disease, fragile-X syndrome, Huntington's chorea, irritable bowel syndrome (IBS), migraine, multiple sclerosis, muscle spasms, pain, chronic pain, acute pain, inflammatory pain, neuropathic pain, diabetic neuropathic pain (DNP), cancer pain, pain related to rheumatic arthritis, allodynia, hyperalgesia, nociceptive pain, post traumatic stress disorder, schizophrenia, positive or cognitive or negative symptoms of schizophrenia, spasticity, Tourette's syndrome, urinary incontinence, vomiting, pruritic conditions, pruritis, sleep disorders, micturition disorders, neuromuscular disorder in the lower urinary tract, gastroesophageal reflux disease (GERD), lower esophageal sphincter (LES) disease, functional gastrointestinal disorders, dyspepsia, regurgitation, respiratory tract infection, bulimia nervosa, chronic laryngitis, asthma, reflux-related asthma, lung disease, eating disorders, obesity and obesity-related disorders, binge eating disorder, agoraphobia, generalized anxiety disorder, obsessive-compulsive disorder, panic disorder, posttraumatic stress disorder, social phobia, substance-induced anxiety disorder, delusional disorder, schizoaffective disorder, schizophreniform disorder, substance-induced psychotic disorder, delirium, or for cognitive enhancement and/or neuroprotection

Such a method wherein the compound is administered in the form of a pharmaceutical composition thereof comprising at least one compound of Formula I in combination with one or more pharmaceutically-acceptable diluents, excipients, or carriers.

Further, the use of at least one compound of Formula I

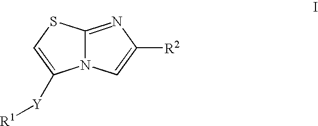

wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroarylC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6a} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
   and optical isomers, polymorphs and pharmaceutically-acceptable acid and base addition salts, hydrates, and solvates thereof;

wherein the term “C₁₋₆alkyl” represents straight or branched chain alkyl groups, examples of such alkyl groups include methyl, ethyl, n-propyl, 2-propyl, n-butyl, 2-butyl, iso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl, and 3-ethylbutyl; the term “C₂₋₆alkenyl” represents straight or branched chain alkenyl groups; the term “cycloC₃₋₁₂alkyl” represents monocyclic, bicyclic or tricyclic alkyl groups including cyclopropyl, cyclobutyl, cyclopentyl, cyclohexyl, bicyclo[2.2.1]heptyl and adamantanyl, optionally substituted by one or more substituents, which may be the same or different, selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl” represents phenyl or naphthyl, wherein the phenyl or naphthyl group is optionally substituted by one or more substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-C₁₋₆alkylamino, C₁₋₆alkylcarbonylamino, C₁₋₆alkylsulfonylamino, pyrrolidinyl, piperidinyl, morpholinyl, and piperazinyl or optionally substituted by C₁₋₆alkylenedioxy; the term “heteroaryl” represents an aromatic 5-6 membered ring comprising one to four heteroatoms selected from oxygen, sulfur and nitrogen or a bicyclic ring system having one 5-6 membered ring comprising one to four heteroatoms selected from oxygen, sulfur and nitrogen fused with a benzene ring or a 5-6 membered ring comprising one to four heteroatoms selected from oxygen, sulfur and nitrogen, wherein the heteroaryl is optionally substituted by one or more substituents, which may be the same or different, selected independently from halogen, trifluoromethyl, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, pyrrolidinyl, piperidinyl, morpholinyl, pyridinyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyi, benzoxazolyl, benzothiazolyl, quinolinyl and isoquinolinyl; the term “heterocyclyl” represents a saturated or unsaturated non-aromatic 3 to 12 membered ring comprising one to four heteroatoms selected from oxygen, sulfur and nitrogen or a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more substituents, which may be the same or different, selected independently from halogen, trifluoromethyl, C₁₋₆ alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, pyrrolidinyl, piperidinyl, morpholinyl, pyridyl, and aryl; examples of such heterocyclyl groups include piperidinyl, morpholinyl or piperazinyl; and the term “halogen” represents fluorine, chlorine, bromine and iodine,

for the manufacturing of a medicament for the prevention and/or treatment of a condition or disease in an animal including a human being which condition or disease is affected or facilitated by the modulatory effect of Group I mGluR modulators or for the manufacturing of a medicament for enhancing cognition.

The compounds of Formula I used according to the present invention for the manufacturing of a medicament have been found to be modulators of Group I mGluR receptors.

Such a medicament may be used for the prevention and/or treatment of AIDS-related dementia, Alzheimer's disease, Creutzfeld-Jakob's syndrome, bovine spongiform encephalopathy (BSE) or other prion related infections, diseases involving mitochondrial dysfunction, diseases involving β-amyloid and/or tauopathy such as Down's syndrome, hepatic encephalopathy, Huntington's disease, motor neuron diseases such as amyotrophic lateral sclerosis (ALS), multiple sclerosis (MS), olivoponto-cerebellar atrophy, post-operative cognitive deficit (POCD), lupus disease, neuronal ceroid lipofuscinosis, neurodegenerative cerebellar ataxias, Parkinson's disease, Parkinson's dementia, mild cognitive impairment, dementia pugilistica, vascular and frontal lobe dementia, cognitive impairment, eye injuries, eye diseases, eye disorders, glaucoma, retinopathy, macular degeneration, head and brain and spinal cord injuries, head and brain and spinal cord trauma, hypoglycaemia, hypoxia, perinatal hypoxia, ischaemia, ischaemia resulting from cardiac arrest or stroke or bypass operations or transplants, convulsions, epilepsy, myoclonic epilepsy, epileptic convulsions, temporal lobe epilepsy, glioma and other tumours, cancer, oral cancer, squamous cell carcinoma (SCC), oral squamous cell carcinoma (SSC), neoplasia, hyperplasia, dysplasia, cancer, carcinoma, sarcoma, oral cancer, squamous cell carcinoma (SCC), oral squamous cell carcinoma (SCC), lung cancer, lung adenocarcinoma, breast cancer, prostate cancer, gastric cancer, liver cancer, colon cancer, colorectal carcinoma, brain tumor, tumor of a nerve tissue, malignant glioma, astroglioma, neuroglioma, neuroblastoma, glioblastoma, medulloblastoma, cancer of skin cells, melanoma, malignant melanoma, epithelial neoplasm, lymphoma, myeloma, Hodgkin's disease, Burkett's lymphoma, leukemia, thymoma, inner ear insult, inner ear insult in tinnitus, tinnitus, sound or drug-induced inner ear insult, sound or drug-induced tinnitus, L-dopa-induced and tardive dyskinesias, L-dopa-induced dyskinesia in Parkinson's disease therapy, chorea, athetosis, stereotypy, ballism, Tic disorder, torticollis spasmodicus, blepharospasm, focal and generalized dystonia, nystagmus, hereditary cerebellar taxias, corticobasale degeneration, tremor, essential tremor, abuse, addiction, nicotine addiction, nicotine abuse, alcohol addiction, alcohol abuse, opiate addiction, opiate abuse, cocaine addiction, cocaine abuse, amphetamine addiction, amphetamine abuse, obesity, anxiety and panic disorders, attention deficit hyperactivity disorder (ADHD), attention deficit syndrome (ADS), restless leg syndrome, hyperactivity in children, autism, dementia, dementia in Alzheimer's disease, dementia in Korsakoff syndrome, vascular dementia, dementia in HIV infections, major depressive disorder or depression, depression resulting from Borna virus infection, and bipolar manic-depressive disorder, drug tolerance, drug tolerance to opioids, movement disorders, dystonia, dyskinesia, L-Dopa-induced dyskinesia, tardive dyskinesia, dyskinesia in Huntington's disease, fragile-X syndrome, Huntington's chorea, irritable bowel syndrome (IBS), migraine, multiple sclerosis, muscle spasms, pain, chronic pain, acute pain, inflammatory pain, neuropathic pain, diabetic neuropathic pain (DNP), cancer pain, pain related to rheumatic arthritis, allodynia, hyperalgesia, nociceptive pain, post traumatic stress disorder, schizophrenia, positive or cognitive or negative symptoms of schizophrenia, spasticity, Tourette's syndrome, urinary incontinence, vomiting, pruritic conditions, pruritis, sleep disorders, micturition disorders, neuromuscular disorder in the lower urinary tract, gastroesophageal reflux disease (GERD), lower esophageal sphincter (LES) disease, functional gastrointestinal disorders, dyspepsia, regurgitation, respiratory tract infection, bulimia nervosa, chronic laryngitis, asthma, reflux-related asthma, lung disease, eating disorders, obesity and obesity-related disorders, binge eating disorder, agoraphobia, generalized anxiety disorder, obsessive-compulsive disorder, panic disorder, posttraumatic stress disorder, social phobia, substance-induced anxiety disorder, delusional disorder, schizoaffective disorder, schizophreniform disorder, substance-induced psychotic disorder, delirium, or for cognitive enhancement and/or neuroprotection

Such a medicament wherein the medicament is for the prevention and/or treatment of addiction, neuropathic pain, L-dopa-induced and tardive dyskinesias, ALS, fragile-X syndrome, Parkinson's disease, anxiety disorders, epilepsy, positive and/or negative symptoms of schizophrenia, cognitive impairment, or for cognitive enhancement and/or neuroprotection.

Further, a pharmaceutical composition comprising, together with one or more pharmaceutically acceptable excipients or vehicles, at least one compound of Formula I

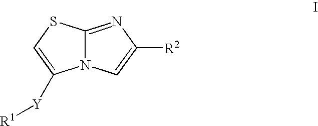

wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroaryC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6a} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;

and optical isomers, polymorphs and pharmaceutically-acceptable acid and base addition salts, hydrates, and solvates thereof;

wherein the term “C₁₋₆alkyl” represents straight or branched chain alkyl groups, examples of such alkyl groups include methyl, ethyl, n-propyl, 2-propyl, n-butyl, 2-butyl, iso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, methylpentyl, 3-methylpentyl, 4-methylpentyl, 2-dimethylbutyl, 3-dimethylbutyl, 2-ethylbutyl, and 3-ethylbutyl; the term “C₂₋₆alkenyl” represents straight or branched chain alkenyl groups; the term “cycloC₃₋₁₂alkyl” represents monocyclic, bicyclic or tricyclic alkyl groups including cyclopropyl, cyclobutyl, cyclopentyl, cyclohexyl, bicyclo[2.2.1]heptyl and adamantanyl, optionally substituted by one or more substituents selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl” represents phenyl or naphthyl, wherein the phenyl or naphthyl group is optionally substituted by one or more substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-C₁₋₆alkylamino, C₁₋₆alkylcarbonylamino, C₁₋₆alkylsulfonylamino, pyrrolidinyl, piperidinyl, morpholinyl, and piperazinyl or optionally substituted by C₁₋₆alkylenedioxy; the term “heteroaryl” represents an aromatic 5-6 membered ring comprising one to four heteroatoms selected from oxygen, sulfur and nitrogen or a bicyclic ring system having one 5-6 membered ring comprising one to four heteroatoms selected from oxygen, sulfur and nitrogen fused with a benzene ring or a 5-6 membered ring comprising one to four heteroatoms selected from oxygen, sulfur and nitrogen, wherein the heteroaryl is optionally substituted by one or more substituents, which may be the same or different, selected independently from halogen, trifluoromethyl, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, pyrrolidinyl, piperidinyl, morpholinyl, pyridyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyl, benzoxazolyl, benzothiazolyl, quinolyl and isoquinolyl; the term “heterocyclyl” represents a saturated or unsaturated non-aromatic 3 to 12 membered ring comprising one to four heteroatoms selected from oxygen, sulfur and nitrogen or a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more substituents selected independently from a halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, pyrrolidinyl, piperidinyl, morpholinyl, pyridyl, and aryl; examples of such heterocyclyl groups include piperidinyl, morpholinyl or piperazinyl; and the term “halogen” represents fluorine, chlorine, bromine and iodine.

Further, a composition comprising a combination of a compound selected from those of Formula I

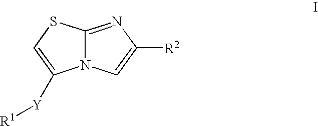

wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroarylC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6a} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;

and optical isomers, polymorphs and pharmaceutically-acceptable acid and base addition salts, hydrates, and solvates thereof;

wherein the term “C₁₋₆alkyl” represents straight or branched chain alkyl groups, examples of such alkyl groups include methyl, ethyl, n-propyl, 2-propyl, n-butyl, 2-butyl, isso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl, and 3-ethylbutyl; the term “C₂₋₆alkenyl” represents straight or branched chain alkenyl groups; the term “cycloC₃₋₁₂alkyl” represents monocyclic, bicyclic or tricyclic alkyl groups including cyclopropyl, cyclobutyl, cyclopentyl, cyclohexyl, bicyclo[2.2.1]heptyl and adamantanyl, optionally substituted by one or more substituents selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl” represents phenyl or naphthyl, wherein the phenyl or naphthyl group is optionally substituted by one or more substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-C₁₋₆alkylamino, C₁₋₆alkylcarbonylamino, C₁₋₆alkylsulfonylamino, pyrrolidinyl, piperidinyl, morpholinyl, and piperazinyl or optionally substituted by C₁₋₆alkylenedioxy; the term “heteroaryl” represents an aromatic 5-6 membered ring comprising one to four heteroatoms selected from oxygen, sulfur and nitrogen or a bicyclic ring system having one 5-6 membered ring comprising one to four heteroatoms selected from oxygen, sulfur and nitrogen fused with a benzene ring or a 5-6 membered ring comprising one to four heteroatoms selected from oxygen, sulfur and nitrogen, wherein the heteroaryl is optionally substituted by one or more substituents, which may be the same or different, selected independently from halogen, trifluoromethyl, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, pyrrolidinyl, piperidinyl, morpholinyl, pyridyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyl, benzoxazolyl, benzothiazolyl, quinolyl and isoquinolyl; the term “heterocyclyl” represents a saturated or unsaturated non-aromatic 3 to 12 membered ring comprising one to four heteroatoms selected from oxygen, sulfur and nitrogen or a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more substituents selected independently from a halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, pyrrolidinyl, piperidinyl, morpholinyl, pyridyl, and aryl; examples of such heterocyclyl groups include piperidinyl, morpholinyl or piperazinyl; and the term “halogen” represents fluorine, chlorine, bromine and iodine;

and an NMDA receptor antagonist.

Such a compostion wherein the NMDA receptor antagonist is selected from memantine and neramexane and pharmaceutically acceptable salts, polymorphs, hydrates, and solvates thereof.

Moreover, a method of providing neuroprotection to a living animal, including a human, comprising the step of administering to a living animal, including a human, a therapeutically effective amount of a composition comprising a compound selected from those of Formula I

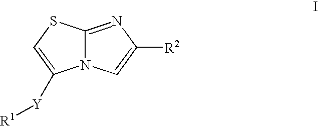

wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroaryC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6a} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;

and optical isomers, polymorphs and pharmaceutically-acceptable acid and base addition salts, hydrates, and solvates thereof;
wherein the term “C₁₋₆alkyl” represents straight or branched chain alkyl groups, examples of such alkyl groups include methyl, ethyl, n-propyl, 2-propyl, n-butyl, 2-butyl, iso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl, and 3-ethylbutyl; the term “C₂₋₆alkenyl” represents straight or branched chain alkenyl groups; the term “cycloC₃₋₁₂alkyl” represents monocyclic, bicyclic or tricyclic alkyl groups including cyclopropyl, cyclobutyl, cyclopentyl, cyclohexyl, bicyclo[2.2.1]heptyl and adamantanyl, optionally substituted by one or more substituents selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl”0 represents phenyl or naphthyl, wherein the phenyl or naphthyl group is optionally substituted by one or more substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-C₁₋₆alkylamino, C₁₋₆alkylcarbonylamino, C₁₋₆alkylsulfonylamino, pyrrolidinyl, piperidinyl, morpholinyl, and piperazinyl or optionally substituted by C₁₋₆alkylenedioxy; the term “heteroaryl” represents an aromatic 5-6 membered ring comprising one to four heteroatoms selected from oxygen, sulfur and nitrogen or a bicyclic ring system having one 5-6 membered ring comprising one to four heteroatoms selected from oxygen, sulfur and nitrogen fused with a benzene ring or a 5-6 membered ring comprising one to four heteroatoms selected from oxygen, sulfur and nitrogen, wherein the heteroaryl is optionally substituted by one or more substituents, which may be the same or different, selected independently from halogen, trifluoromethyl, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, pyrrolidinyl, piperidinyl, morpholinyl, pyridyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyl, benzoxazolyl, benzothiazolyl, quinolyl and isoquinolyl; the term “heterocyclyl” represents a saturated or unsaturated non-aromatic 3 to 12 membered ring comprising one to four heteroatoms selected from oxygen, sulfur and nitrogen or a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more substituents selected independently from a halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, pyrrolidinyl, piperidinyl, morpholinyl, pyridyl, and aryl; examples of such heterocyclyl groups include piperidinyl, morpholinyl or piperazinyl; and the term “halogen” represents fluorine, chlorine, bromine and iodine;
   and an NMDA receptor antagonist.

Further, the use of a composition comprising a compound selected from those of Formula I

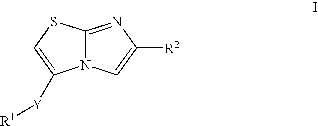

wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroarylC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6b} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;

and optical isomers, polymorphs and pharmaceutically-acceptable acid and base addition salts, hydrates, and solvates thereof;
wherein the term “C₁₋₆alkyl” represents straight or branched chain alkyl groups, examples of such alkyl groups include methyl, ethyl, n-propyl, 2-propyl, n-butyl, 2-butyl, iso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl, and 3-ethylbutyl; the term “C₂₋₆alkenyl” represents straight or branched chain alkenyl groups; the term “cycloC₃₋₁₂alkyl” represents monocyclic, bicyclic or tricyclic alkyl groups including cyclopropyl, cyclobutyl, cyclopentyl, cyclohexyl, bicyclo[2.2.1]heptyl and adamantanyl, optionally substituted by one or more substituents selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl” represents phenyl or naphthyl, wherein the phenyl or naphthyl group is optionally substituted by one or more substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-C₁₋₆alkylamino, C₁₋₆alkylcarbonylamino, C₁₋₆alkylsulfonylamino, pyrrolidinyl, piperidinyl, morpholinyl, and piperazinyl or optionally substituted by C₁₋₆alkylenedioxy; the term “heteroaryl” represents an aromatic 5-6 membered ring comprising one to four heteroatoms selected from oxygen, sulfur and nitrogen or a bicyclic ring system having one 5-6 membered ring comprising one to four heteroatoms selected from oxygen, sulfur and nitrogen fused with a benzene ring or a 5-6 membered ring comprising one to four heteroatoms selected from oxygen, sulfur and nitrogen, wherein the heteroaryl is optionally substituted by one or more substituents, which may be the same or different, selected independently from halogen, trifluoromethyl, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, pyrrolidinyl, piperidinyl, morpholinyl, pyridyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyl, benzoxazolyl, benzothiazolyl, quinolyl and isoquinolyl; the term “heterocyclyl” represents a saturated or unsaturated non-aromatic 3 to 12 membered ring comprising one to four heteroatoms selected from oxygen, sulfur and nitrogen or a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more substituents selected independently from a halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, pyrrolidinyl, piperidinyl, morpholinyl, pyridyl, and aryl; examples of such heterocyclyl groups include piperidinyl, morpholinyl or piperazinyl; and the term “halogen” represents fluorine, chlorine, bromine and iodine;
   and an NMDA receptor antagonist for the manufacture of a medicament to provide neuroprotection in an animal, including a human.

In an additional embodiment of the present invention, the compounds of Formula I may be represented by Formula I′:

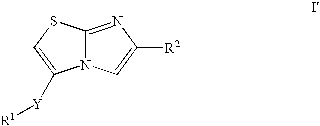

wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroarylC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6a} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
   and optical isomers, polymorphs and pharmaceutically-acceptable acid and base addition salts, hydrates, and solvates thereof;

wherein the term “C₁₋₆alkyl” represents straight or branched chain alkyl groups, examples of such alkyl groups include methyl, ethyl, n-propyl, 2-propyl, n-butyl, 2-butyl, iso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl, and 3-ethylbutyl; the term “C₂₋₆alkenyl” represents straight or branched chain alkenyl groups; the term “cycloC₃₋₁₂alkyl” represents monocyclic, bicyclic or tricyclic alkyl groups including cyclopropyl, cyclobutyl, cyclopentyl, cyclohexyl, bicyclo[2.2.1]heptyl and adamantanyl, wherein the cyclic alkyl group is optionally substituted by one or more substituents, which may be the same or different, selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl” represents phenyl or naphthyl, wherein the phenyl or naphthyl group is optionally substituted by one or more substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylamino, di-C₁₋₆alkylamino, C₁₋₆alkylcarbonylamino, piperidinyl, morpholinyl, and piperazinyl or optionally substituted by C₁₋₆alkylenedioxy; the term “heteroaryl” represents an aromatic 5-6 membered ring comprising one to four heteroatoms selected from oxygen, sulfur and nitrogen, and a bicyclic ring system having one 5-6 membered ring comprising one to four heteroatoms selected from oxygen, sulfur and nitrogen fused with a benzene ring or a 5-6 membered ring comprising one to four heteroatoms selected from oxygen, sulfur and nitrogen, wherein the heteroaryl is optionally substituted by one or more substituents, which may be the same or different selected independently from halogen, trifluoromethyl, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, piperidinyl, morpholinyl, pyridyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyl, benzoxazolyl, benzothiazolyl, quinolyl and isoquinolyl; the term “heterocyclyl” represents a saturated or unsaturated non-aromatic 3 to 12 membered ring comprising one to four heteroatoms selected from oxygen, sulfur and nitrogen, and a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more substituents selected independently from a halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, piperidinyl, morpholinyl, pyridyl, and aryl; examples of such heterocyclyl groups include piperidinyl, morpholinyl and piperazinyl; and the term “halogen” represents fluorine, chlorine, bromine and iodine.

Such a compound of Formula I′ wherein Y represents a single bond, R¹ represents aryl or heteroaryl, and R² represents cycloC₃₋₁₂alkyl.

Such a compound of Formula I′ wherein R² represents branched C₁₋₆alkyl.

Such a compound of Formula I′ wherein R² represents 2-propyl, 2-butyl, iso-butyl, tert-butyl, 2-pentyl, 3-pentyl, iso-pentyl, 2-methylbutyl, tert-amyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl or 3-ethylbutyl.

Such a compound of Formula I′ wherein R² represents branched C₁₋₆alkyl, Y represents a single bond and
R¹ represents aryl optionally substituted by one or more substituents, which may be the same or different, selected independently from C₁₋₆alkyl, C₁₋₆alkoxy, halogen, and C₁₋₆alkoxycarbonyl.

Such a compound of Formula I′ wherein R² represents branched C₁₋₆alkyl, Y represents a single bond and R¹ represents phenyl optionally substituted by one or more substituents selected from C₁₋₆alkyl, C₁₋₆alkoxy, halogen, and C₁₋₆alkoxycarbonyl.

Such a compound of Formula I′ wherein
R² represents branched C₁₋₆alkyl, including 2-propyl, 2-butyl, iso-butyl, tert-butyl, 2-pentyl, 3-pentyl, iso-pentyl, 2-methylbutyl, tert-amyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl or 3-ethylbutyl;
Y represents a single bond; and
R¹ represents aryl, including phenyl, optionally substituted by one or more substituents selected independently from C₁₋₆alkyl, including methyl, C₁₋₆alkoxy, including methoxy, halogen, including bromine, C₁₋₆alkoxycarbonyl, including methoxycarbonyl.

Further, a pharmaceutical composition comprising, together with one or more pharmaceutically acceptable excipients or vehicles, at least one compound of Formula I′

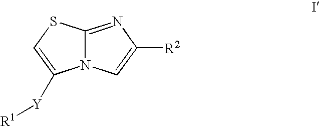

wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroarylC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6a} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;

and optical isomers, polymorphs and pharmaceutically-acceptable acid and base addition salts, hydrates, and solvates thereof;
wherein the term “C₁₋₆alkyl” represents straight or branched chain alkyl groups, examples of such alkyl groups include methyl, ethyl, n-propyl, 2-propyl, n-butyl, 2-butyl, iso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl, and 3-ethylbutyl; the term “C₂₋₆alkenyl” represents straight or branched chain alkenyl groups; the term “cycloC₃₋₁₂alkyl” represents monocyclic, bicyclic or tricyclic alkyl groups including cyclopropyl, cyclobutyl, cyclopentyl, cyclohexyl, bicyclo[2.2.1]heptyl and adamantanyl, optionally substituted by one or more substituents selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl” represents phenyl or naphthyl, wherein the phenyl or naphthyl group is optionally substituted by one or more substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylamino, di-C₁₋₆alkylamino, C₁₋₆alkylcarbonylamino, piperidinyl, morpholinyl, and piperazinyl or optionally substituted by C₁₋₆alkylenedioxy; the term “heteroaryl” represents an aromatic 5-6 membered ring comprising one to four heteroatoms selected from oxygen, sulfur and nitrogen, and a bicyclic ring system having one 5-6 membered ring comprising one to four heteroatoms selected from oxygen, sulfur and nitrogen, fused with a benzene ring or a 5-6 membered ring comprising one to four heteroatoms selected from oxygen, sulfur and nitrogen, wherein the heteroaryl is optionally substituted by one or more substituents, which may be the same or different, selected independently from halogen, trifluoromethyl, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, piperidinyl, morpholinyl, pyridyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyl, benzoxazolyl, benzothiazolyl, quinolyl and isoquinolyl; the term “heterocyclyl” represents a saturated or unsaturated non-aromatic 3 to 12 membered ring comprising one to four heteroatoms selected from oxygen, sulfur and nitrogen, and a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more substituents selected independently from a halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, piperidinyl, morpholinyl, pyridyl, and aryl; examples of such heterocyclyl groups include piperidinyl, morpholinyl or piperazinyl; and the term “halogen” represents fluorine, chlorine, bromine and iodine.

Moreover, a method for treating or preventing a condition or disease associated with abnormal glutamate neurotransmission or a method for modulating Group I mGluR receptors to achieve therapeutic benefit, or a method for enhancing cognition, such method comprising administering to a living animal, including a human, a therapeutically effective amount of a compound selected from those of Formula I′

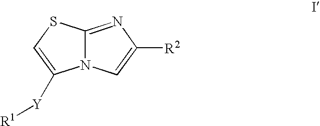

wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroarylC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6a} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
   and optical isomers, polymorphs and pharmaceutically-acceptable acid and base addition salts, hydrates, and solvates thereof;

wherein the term “C₁₋₆alkyl” represents straight or branched chain alkyl groups, examples of such alkyl groups include methyl, ethyl, n-propyl, 2-propyl, n-butyl, 2-butyl, iso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl, and 3-ethylbutyl; the term “C₂₋₆alkenyl” represents straight or branched chain alkenyl groups; the term “cycloC₃₋₁₂alkyl” represents monocyclic, bicyclic or tricyclic alkyl groups including cyclopropyl, cyclobutyl, cyclopentyl, cyclohexyl, bicyclo[2.2.1]heptyl and adamantanyl, wherein the monocyclic, bicyclic or tricyclic alkyl group is optionally substituted by one or more substituents, which may be the same or different, selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl” represents phenyl, or naphthyl, wherein the phenyl or naphthyl group is optionally substituted by one or more substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylamino, di-C₁₋₆alkylamino, C₁₋₆alkylcarbonylamino, pyrrolidinyl, piperidinyl, morpholinyl, and piperazinyl or optionally substituted by C₁₋₆alkylenedioxy; the term “heteroaryl” represents an aromatic 5-6 membered ring comprising one to four heteroatoms selected from oxygen, sulfur and nitrogen, and a bicyclic ring system having one 5-6 membered ring comprising one to four heteroatoms selected from oxygen, sulfur and nitrogen fused with a benzene ring or a 5-6 membered ring comprising one to four heteroatoms selected from oxygen, sulfur and nitrogen, wherein the heteroaryl is optionally substituted by one or more substituents, which may be the same or different, selected independently from halogen, trifluoromethyl, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, pyrrolidinyl, piperidinyl, morpholinyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyl, benzoxazolyl, benzothiazolyl, quinolinyl and isoquinolinyl; the term “heterocyclyl” represents a saturated or unsaturated non-aromatic 3 to 12 membered ring comprising one to four heteroatoms selected from oxygen, sulfur and nitrogen, and a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more substituents selected independently from a halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, pyrrolidinyl, piperidinyl, morpholinyl, pyridinyl, and aryl; examples of such heterocyclyl groups include piperidinyl, morpholinyl or piperazinyl; and the term “halogen” represents fluorine, chlorine, bromine and iodine.

Such a method wherein the condition associated with abnormal glutamate neurotransmission, or wherein modulation of mGluR receptors results in therapeutic benefit, is selected from: AIDS-related dementia, Alzheimer's disease, Creutzfeld-Jakob's syndrome, bovine spongiform encephalopathy (BSE) or other prion related infections, diseases involving mitochondrial dysfunction, diseases involving β-amyloid and/or tauopathy such as Down's syndrome, hepatic encephalopathy, Huntington's disease, motor neuron diseases such as amyotrophic lateral sclerosis (ALS), multiple sclerosis (MS), olivoponto-cerebellar atrophy, post-operative cognitive deficit (POCD), Parkinson's disease, Parkinson's dementia, mild cognitive impairment, dementia pugilisitca, vascular and frontal lobe dementia, cognitive impairment, eye injuries or diseases, glaucoma, retinopathy, macular degeneration, head and spinal cord injuries/trauma, hypoglycaemia, hypoxia, perinatal hypoxia, ischaemia resulting from cardiac arrest or stroke or bypass operations or transplants, convulsions, glioma and other tumours, inner ear insult, inner ear insult in tinnitus, sound or drug-induced inner ear insult, L-dopa-induced and tardive dyskinesias, addiction, nicotine addiction, alcohol addiction, opiate addiction, cocaine addiction, amphetamine addiction, obesity addiction, anxiety and panic disorders, attention deficit hyperactivity disorder (ADHD), restless leg syndrome, hyperactivity in children, autism, convulsions/epilepsy, dementia, dementia in Alzheimer's disease, dementia in Korsakoff syndrome, vascular dementia, dementia in HIV infections, major depressive disorder or depression, depression resulting from Borna virus infection, and bipolar manic-depressive disorder, drug tolerance, drug tolerance to opioids, movement disorders, dystonia, dyskinesia, L-Dopa-induced dyskinesia, tardive dyskinesia or dyskinesia in Huntington's disease, fragile-X syndrome, Huntington's chorea, irritable bowel syndrome (IBS), migraine, multiple sclerosis, muscle spasms, pain, chronic pain and acute pain, inflammatory pain, neuropathic pain, allodynia, hyperalgesia, nociceptive pain, Parkinson's disease, post traumatic stress disorder, schizophrenia, positive or cognitive or negative symptoms of schizophrenia, spasticity, Tourette's syndrome, urinary incontinence and vomiting, pruritic conditions, pruritis, sleep disorders, micturition disorders, neuromuscular disorder in the lower urinary tract, gastroesophageal reflux disease (GERD), lower esophageal sphincter (LES) disease, functional gastrointestinal disorders, dyspepsia, regurgitation, respiratory tract infection, bulimia nervosa, chronic laryngitis, asthma, reflux-related asthma, lung disease, eating disorders, obesity and obesity-related disorders, agoraphobia, generalized anxiety disorder, obsessive-compulsive disorder, panic disorder, posttraumatic stress disorder, social phobia, substance-induced anxiety disorder, delusional disorder, schizoaffective disorder, schizophreniform disorder, substance-induced psychotic disorder, delirium, or for cognitive enhancement and/or neuroprotection.

Further the use of at least one compound of Formula I′

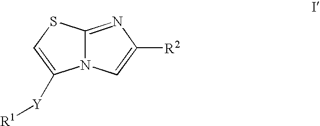

wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroarylC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6a} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
   and optical isomers, polymorphs and pharmaceutically-acceptable acid and base addition salts, hydrates, and solvates thereof;

wherein the term “C₁₋₆alkyl” represents straight or branched chain alkyl groups, examples of such alkyl groups include methyl, ethyl, n-propyl, 2-propyl, n-butyl, 2-butyl, iso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, 3-methylpentyl, 4-methylpentyl, 2-dimethylbutyl, 3-dimethylpentyl, 2-ethylbutyl, and 3-ethylbutyl; the term “C₂₋₆alkenyl” represents straight or branched chain alkenyl groups; the term “cycloC₃₋₁₂alkyl” represents monocyclic, bicyclic or tricyclic alkyl groups including cyclopropyl, cyclobutyl, cyclopentyl, cyclohexyl, bicyclo[2.2.1]heptyl and adamantanyl, optionally substituted by one or more substituents, which may be the same or different, selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl” represents phenyl or naphthyl, wherein the phenyl or naphthyl group is optionally substituted by one or more substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylamino, di-C₁₋₆alkylamino, C₁₋₆alkylcarbonylamino, pyrrolidinyl, piperidinyl, morpholinyl, and piperazinyl or optionally substituted by C₁₋₆alkylenedioxy; the term “heteroaryl” represents an aromatic 5-6 membered ring comprising one to four heteroatoms selected from oxygen, sulfur and nitrogen, and a bicyclic ring system having one 5-6 membered ring comprising one to four heteroatoms selected from oxygen, sulfur and nitrogen fused with a benzene ring or a 5-6 membered ring comprising one to four heteroatoms selected from oxygen, sulfur and nitrogen, wherein the heteroaryl is optionally substituted by one or more substituents, which may be the same or different, selected independently from halogen, trifluoromethyl, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, pyrrolidinyl, piperidinyl, morpholinyl, pyridinyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyl, benzoxazolyl, benzothiazolyl, quinolinyl and isoquinolinyl; the term “heterocyclyl” represents a saturated or unsaturated non-aromatic 3 to 12 membered ring comprising one to four heteroatoms selected from oxygen, sulfur and nitrogen, and a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more substituents, which may be the same or different, selected independently from halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, pyrrolidinyl, piperidinyl, morpholinyl, pyridyl, and aryl; examples of such heterocyclyl groups include piperidinyl, morpholinyl or piperazinyl; and the term “halogen” represents fluorine, chlorine, bromine and iodine,

for the manufacturing of a medicament for the prevention and/or treatment of a condition or disease in an animal including a human being which condition or disease is affected or facilitated by the modulatory effect of Group I mGluR modulators or for the manufacturing of a medicament for enhancing cognition.

Such a use wherein the condition associated with abnormal glutamate neurotransmission, or wherein modulation of mGluR receptors results in therapeutic benefit, is selected from: AIDS-related dementia, Alzheimer's disease, Creutzfeld-Jakob's syndrome, bovine spongiform encephalopathy (BSE) or other prion related infections, diseases involving mitochondrial dysfunction, diseases involving β-amyloid and/or tauopathy such as Down's syndrome, hepatic encephalopathy, Huntington's disease, motor neuron diseases such as amyotrophic lateral sclerosis (ALS), multiple sclerosis (MS), olivoponto-cerebellar atrophy, post-operative cognitive deficit (POCD), Parkinson's disease, Parkinson's dementia, mild cognitive impairment, dementia pugilisitca, vascular and frontal lobe dementia, cognitive impairment, eye injuries or diseases, glaucoma, retinopathy, macular degeneration, head and spinal cord injuries/trauma, hypoglycaemia, hypoxia, perinatal hypoxia, ischaemia resulting from cardiac arrest or stroke or bypass operations or transplants, convulsions, glioma and other tumours, inner ear insult, inner ear insult in tinnitus, sound or drug-induced inner ear insult, L-dopa-induced and tardive dyskinesias, addiction, nicotine addiction, alcohol addiction, opiate addiction, cocaine addiction, amphetamine addiction, obesity addiction, anxiety and panic disorders, attention deficit hyperactivity disorder (ADHD), restless leg syndrome, hyperactivity in children, autism, convulsions/epilepsy, dementia, dementia in Alzheimer's disease, dementia in Korsakoff syndrome, vascular dementia, dementia in HIV infections, major depressive disorder or depression, depression resulting from Borna virus infection, and bipolar manic-depressive disorder, drug tolerance, drug tolerance to opioids, movement disorders, dystonia, dyskinesia, L-Dopa-induced dyskinesia, tardive dyskinesia or dyskinesia in Huntington's disease, fragile-X syndrome, Huntington's chorea, irritable bowel syndrome (IBS), migraine, multiple sclerosis, muscle spasms, pain, chronic pain and acute pain, inflammatory pain, neuropathic pain, allodynia, hyperalgesia, nociceptive pain, Parkinson's disease, post traumatic stress disorder, schizophrenia, positive or cognitive or negative symptoms of schizophrenia, spasticity, Tourette's syndrome, urinary incontinence and vomiting, pruritic conditions, pruritis, sleep disorders, micturition disorders, neuromuscular disorder in the lower urinary tract, gastroesophageal reflux disease (GERD), lower esophageal sphincter (LES) disease, functional gastrointestinal disorders, dyspepsia, regurgitation, respiratory tract infection, bulimia nervosa, chronic laryngitis, asthma, reflux-related asthma, lung disease, eating disorders, obesity and obesity-related disorders, agoraphobia, generalized anxiety disorder, obsessive-compulsive disorder, panic disorder, posttraumatic stress disorder, social phobia, substance-induced anxiety disorder, delusional disorder, schizoaffective disorder, schizophreniform disorder, substance-induced psychotic disorder, delirium, or for cognitive enhancement and/or neuroprotection.

Specific compounds of Formula I within the present invention include but are not limited to:
6-Adamantan-1-yl-3-(2,5-dimethoxyphenyl)-imidazo[2,1-b]thiazole,
6-Adamantan-1-yl-3-(2,5-difluorophenyl)-imidazo[2,1-b]thiazole,
6-Adamantan-1-yl-3-(4-methylphenyl)-imidazo[2,1-b]thiazole,
6-Adamantan-1-yl-3-(2,4-dimethylphenyl)-imidazo[2,1-b]thiazole,
6-Adamantan-1-yl-3-(2,4-dimethoxyphenyl)-imidazo[2,1-b]thiazole,
6-Adamantan-1-yl-3-benzo[1,3]dioxol-5-yl-imidazo[2,1-b]thiazole,
6-Adamantan-1-yl-3-(3,4-dimethoxyphenyl)-imidazo[2,1-b]thiazole,
6-Adamantan-1-yl-3-benzofuran-2-yl-imidazo[2,1-b]thiazole,
6-Adamantan-1-yl-3-(4-fluorophenyl)-imidazo[2,1-b]thiazole,
6-Adamantan-1-yl-3-thiophen-2-yl-imidazo[2,1-b]thiazole,
6-Adamantan-1-yl-3-(4-methoxy-3-methyl-phenyl)-imidazo[2,1-b]thiazole,
6-Adamantan-1-yl-3-(3-trifluoromethoxyphenyl)-imidazo[2,1-b]thiazole,
6-(Adamantan-1-yl)-3-(2,4,6-trimethylphenyl)-imidazo[2,1-b]thiazole,
6-Adamantan-1-yl-3-(2-trifluoromethylphenyl)-imidazo[2,1-b]thiazole,
6-Adamantan-1-yl-3-(2,5-diethylphenyl)-imidazo[2,1-b]thiazole,
6-Cyclohexyl-3-(2,5-difluorophenyl)-imidazo[2,1-b]thiazole,
3-(6-Adamantan-1-yl-imidazo[2,1-b]thiazol-3-yl)-5-methoxy-1,2-dimethyl-1H-indole,
6-Adamantan-1-yl-3-(3-bromophenyl)-imidazo[2,1-b]thiazole,
6-Adamantan-1-yl-3-(3-acetylaminophenyl)-imidazo[2,1-b]thiazole,
6-Adamantan-1-yl-3-(3-aminophenyl)-imidazo[2,1-b]thiazole,
3-(2,5-Dimethoxyphenyl)-6-(1-methyl-1-phenylethyl)-imidazo[2,1-b]thiazole,
3-(2,5-Dimethylphenyl)-6-(1-methyl-1-phenylethyl)-imidazo[2,1-b]thiazole,
3-(2,5-Dimethoxyphenyl)-6-piperidin-1-yl-imidazo[2,1-b]thiazole,
6-Azepan-1-yl-3-(2,5-dimethoxyphenyl)-imidazo[2,1-b]thiazole,
3-(2,4-Dimethoxyphenyl)-6-[1-(3-fluorophenyl)-1-methyl-ethyl]-imidazo[2,1-b]thiazole,
3-(2,4-Dimethoxyphenyl)-6-(1-methyl-1-phenyl-ethyl)-imidazo[2,1-b]thiazole,
6-Adamantan-1-yl-3-(3-dimethylaminophenyl)-imidazo[2,1-b]thiazole,
6-Adamantan-1-yl-3-(1,2,5-trimethyl-1H-pyrrol-3-yl)-imidazo[2,1-b]thiazole,
6-Adamantan-1-yl-3-(1-methyl-1H-pyrrol-2-yl)-imidazo[2,1-b]thiazole,
6-Adamantan-1-yl-3-(3-trifluoromethylphenyl)-imidazo[2,1-b]thiazole,
6-Adamantan-1-yl-3-(2,5-dimethylthiophen-3-yl)-imidazo[2,1-b]thiazole,
6-Cyclohexyl-3-(2,5-dimethoxyphenyl)-imidazo[2,1-b]thiazole,
6-Adamantan-1-yl-3-(2,5-dimethylphenyl)-imidazo[2,1-b]thiazole,
4-(6-Adamantan-1-yl-imidazo[2,1-b]thiazol-3-yl)-benzonitrile,
4-(6-Adamantan-1-yl-imidazo[2,1-b]thiazol-3-yl)benzene-1,3-diol,
3-(6-Adamantan-1-yl-imidazo[2,1-b]thiazol-3-yl)phenyl]-methanol,
3-(2,5-Dimethoxyphenyl)-6-[1-(4-fluorophenyl)1-methyl-ethyl]-imidazo[2,1-b]thiazole,
6-Adamantan-1-yl-3-(3-methoxyphenyl)-imidazo[2,1-b]thiazole,
3-(6-Adamantan-1-yl-imidazo[2,1-b]thiazol-3-yl)phenol,
Acetic acid 3-(6-adamantan-1-yl-imidazo[2,1-b]thiazol-3-yl)phenylester,
5-(6-Adamantan-1-yl-imidazo[2,1-b]thiazol-3-yl)-2-methoxy-phenylamine,
6-Adamantan-1-yl-3-(4-methoxyphenyl)-imidazo[2,1-b]thiazole,
3-(3-bromophenyl)-6-tert-butyl-imidazo[2,1-b]thiazole,
6-tert-Butyl-3-(2,4-dimethylphenyl)-imidazo[2,1-b]thiazole,
6-tert-Butyl-3-(p-tolyl)-imidazo[2,1-b]thiazole,
6-tert-Butyl-3-(3-methoxyphenyl)-imidazo[2,1-b]thiazole,
6-tert-Butyl-3-(2,5-dimethoxyphenyl)-imidazo[2,1-b]thiazole,
6-Adamantan-1-yl-3-(6-aminopyridin-3-yl)imidazo[2,1-b]thiazole,
6-Adamantan-1-yl-3-pyridin-3-yl-imidazo[2,1-b]thiazole,
6-Adamantan-1-yl-3-(6-methoxy-pyridin-3-yl)-imidazo[2,1-b]thiazole,
6-Adamantan-1-yl-3-(2-fluoro-pyridin-3-yl)-imidazo[2,1-b]thiazole,
6-Adamantan-1-yl-3-pyridin-4-yl-imidazo[2,1-b]thiazole,
6-Adamantan-1-yl-3-(2,4-dimethoxy-pyrimidin-5-yl)-imidazo[2,1-b]thiazole,
8-[3-(3-Methoxyphenyl)-imidazo[2,1-b]thiazol-6-yl]-8-aza-spiro[4.5]decane,
8-[3-(3-Methoxyphenyl)-imidazo[2,1-b]thiazol-6-yl]-1,4-dioxa-8-aza-spiro[4.5]decane,
8-{1-[3-(3-Methoxyphenyl)-imidazo[2,1-b]thiazol-6-yl]-1-methyl-ethyl}-8-aza-spiro[4.5]decane,
8-{1-[3-(3-Methoxy-phenyl)-imidazo[2,1-b]thiazol-6-yl]-1-methyl-ethyl}-1,4-dioxa-8-aza-spiro[4.5]decane,
6-Adamantan-1-yl-3-(3,4-difluorophenyl)-imidazo[2,1-b]thiazole,
3-(6-tert-Butyl-imidazo[2,1-b]thiazol-3-yl)benzoic acid methyl ester,
Acetic acid, 3-(6-Adamantan-1-yl-imidazo[2,1-b]thiazol-3-yl)benzyl ester,
2-(6-Adamantan-1-yl-imidazo[2,1-b]thiazol-3-yl)phenylamine,
N[3-(6-Adamantan-1-yl-imidazo[2,1-b]thiazol-3-yl)-phenyl]-methanesulfamide,
3-(6-Adamantan-1-yl-imidazo[2,1-b]thiazol-3-yl)-benzonitrile,
Acetic acid, 3-acetoxy-4-(6-Adamantan-1-yl-imidazo[2,1-b]thiazol-3-yl)phenyl ester,
Acetic acid, 2-acetoxy-4-(6-Adamantan-1-yl-imidazo[2,1-b]thiazol-3-yl)phenyl ester,
4-(6-Adamantan-1-yl-imidazo[2,1-b]thiazol-3-yl)benzene-1,2-diol,
Acetic acid, 4-acetoxy-3-(6-Adamantan-1-yl-imidazo[2,1-b]thiazol-3-yl)phenyl ester,
2-(6-Adamantan-1-yl-imidazo[2,1-b]thiazol-3-yl)benzene-1,4-diol,
6-Adamantan-1-yl-3-pyridin-2-yl-imidazo[2,1-b]thiazole,
6-Adamantan-1-yl-3-(4-amino-3-methoxyphenyl)imidazo[2,1-b]thiazole,
6-Adamantan-1-yl-3-(3-chlorophenyl)-imidazo[2,1-b]thiazole,
3-(6-Adamantan-1-yl-imidazo[2,1-b]thiazol-3-yl)benzoic acid methyl ester,
6-Adamantan-1-yl-3-(3,5-dimethoxyphenyl)-imidazo[2,1-b]thiazole,
N-[5-(6-Adamantan-1-yl-imidazo[2,1-b]thiazol-3-yl)-2-methoxy-phenyl]-acetamide,
6-Adamantan-1-yl-2-phenyl-imidazo[2,1-b]thiazole,
6-Adamantan-1-yl-3-(2,5-dimethoxyphenyl)-imidazo[2,1-b]thiazole hydrochloride,
6-Adamantan-1-yl-3-(2,5-difluorophenyl)-imidazo[2,1-b]thiazole hydrochloride,
6-Adamantan-1-yl-3-(4-methylphenyl)-imidazo[2,1-b]thiazole hydrochloride,
6-Adamantan-1-yl-3-(2,4-dimethylphenyl)-imidazo[2,1-b]thiazole hydrochloride,
6-Adamantan-1-yl-3-(2,4-dimethoxyphenyl)-imidazo[2,1-b]thiazole hydrochloride,
6-Adamantan-1-yl-3-benzo[1,3]dioxol-5-yl-imidazo[2,1-b]thiazole hydrochloride,
6-Adamantan-1-yl-3-(3,4-dimethoxyphenyl)-imidazo[2,1-b]thiazole hydrochloride,
6-Adamantan-1-yl-3-benzofuran-2-yl-imidazo[2,1-b]thiazole hydrochloride,
6-Adamantan-1-yl-3-(4-fluorophenyl)-imidazo[2,1-b]thiazole hydrochloride,
6-Adamantan-1-yl-3-thiophen-2-yl-imidazo[2,1-b]thiazole hydrobromide,
6-Adamantan-1-yl-3-(4-methoxy-3-methyl-phenyl)-imidazo[2,1-b]thiazole hydrochloride,
6-Adamantan-1-yl-3-(3-trifluoromethoxyphenyl)-imidazo[2,1-b]thiazole hydrochloride,
6-(Adamantan-1-yl)-3-(2,4,6-trimethylphenyl)-imidazo[2,1-b]thiazole hydrochloride,
6-Adamantan-1-yl-3-(2-trifluoromethylphenyl)-imidazo[2,1-b]thiazole hydrochloride,
6-Adamantan-1-yl-3-(2,5-diethylphenyl)-imidazo[2,1-b]thiazole hydrochloride,
6-Cyclohexyl-3-(2,5-difluorophenyl)-imidazo[2,1-b]thiazole hydrochloride,
3-(6-Adamantan-1-yl-imidazo[2,1-b]thiazol-3-yl)-5-methoxy-1,2-dimethyl-1H-indole hydrochloride,
6-Adamantan-1-yl-3-(3-bromophenyl)-imidazo[2,1-b]thiazole hydrochloride,
6-Adamantan-1-yl-3-(3-acetylaminophenyl)-imidazo[2,1-b]thiazole hydrochloride,
6-Adamantan-1-yl-3-(3-aminophenyl)-imidazo[2,1-b]thiazole hydrochloride,
3-(2,5-Dimethoxyphenyl)-6-(1-methyl-1-phenylethyl)-imidazo[2,1-b]thiazole hydrochloride,
3-(2,5-Dimethylphenyl)-6-(1-methyl-1-phenylethyl)-imidazo[2,1-b]thiazole hydrochloride,
3-(2,5-Dimethoxyphenyl)-6-piperidin-1-yl-imidazo[2,1-b]thiazole hydrochloride,
6-Azepan-1-yl-3-(2,5-dimethoxyphenyl)-imidazo[2,1-b]thiazole hydrochloride,
3-(2,4-Dimethoxyphenyl)-6-[1-(3-fluorophenyl)-1-methyl-ethyl]-imidazo[2,1-b]thiazole hydrochloride,
3-(2,4-Dimethoxyphenyl)-6-(1-methyl-1-phenyl-ethyl)-imidazo[2,1-b]thiazole hydrochloride,
6-Adamantan-1-yl-3-(3-dimethylaminophenyl)-imidazo[2,1-b]thiazole hydrochloride,
6-Adamantan-1-yl-3-(1,2,5-trimethyl-1H-pyrrol-3-yl)-imidazo[2,1-b]thiazole hydrochloride,
6-Adamantan-1-yl-3-(1-methyl-1H-pyrrol-2-yl)-imidazo[2,1-b]thiazole hydrochloride,
6-Adamantan-1-yl-3-(3-trifluoromethylphenyl)-imidazo[2,1-b]thiazole hydrochloride,
6-Adamantan-1-yl-3-(2,5-dimethylthiophen-3-yl)-imidazo[2,1-b]thiazole hydrochloride,
6-Cyclohexyl-3-(2,5-dimethoxyphenyl)-imidazo[2,1-b]thiazole hydrochloride,
6-Adamantan-1-yl-3-(2,5-dimethylphenyl)-imidazo[2,1-b]thiazole hydrochloride,
4-(6-Adamantan-1-yl-imidazo[2,1-b]thiazol-3-yl)-benzonitrile hydrochloride,
4-(6-Adamantan-1-yl-imidazo[2,1-b]thiazol-3-yl)benzene-1,3-diol hydrochloride,
3-(6-Adamantan-1-yl-imidazo[2,1-b]thiazol-3-yl)phenyl]-methanol hydrochloride,
3-(2,5-Dimethoxyphenyl)-6-[1-(4-fluorophenyl) 1 -methyl-ethyl]-imidazo[2,1-b]thiazole hydrochloride,
6-Adamantan-1-yl-3-(3-methoxyphenyl)-imidazo[2,1-b]thiazole hydrochloride,
3-(6-Adamantan-1-yl-imidazo[2,1-b]thiazol-3-yl)phenol hydrochloride,
Acetic acid 3-(6-adamantan-1-yl-imidazo[2,1-b]thiazol-3-yl)phenylester hydrochloride,
5-(6-Adamantan-1-yl-imidazo[2,1-b]thiazol-3-yl)-2-methoxy-phenylamine hydrochloride,
6-Adamantan-1-yl-3-(4-methoxyphenyl)-imidazo[2,1-b]thiazole hydrochloride,
3-(3-bromophenyl)-6-tert-butyl-imidazo[2,1-b]thiazole hydrochloride,
6-tert-Butyl-3-(2,4-dimethylphenyl)-imidazo[2,1-b]thiazole hydrochloride,
6-tert-Butyl-3-(p-tolyl)-imidazo[2,1-b]thiazole hydrochloride,
6-tert-Butyl-3-(3-methoxyphenyl)-imidazo[2,1-b]thiazole hydrochloride,
6-tert-Butyl-3-(2,5-dimethoxyphenyl)-imidazo[2,1-b]thiazole hydrochloride,
6-Adamantan-1-yl-3-(6-aminopyridin-3-yl)imidazo[2,1-b]thiazole hydrochloride,
6-Adamantan-1-yl-3-pyridin-3-yl-imidazo[2,1-b]thiazole hydrochloride,
6-Adamantan-1-yl-3-(6-methoxy-pyridin-3-yl)-imidazo[2,1-b]thiazole hydrochloride,
6-Adamantan-1-yl-3-(2-fluoro-pyridin-3-yl)-imidazo[2,1-b]thiazole hydrochloride,
6-Adamantan-1-yl-3-pyridin-4-yl-imidazo[2,1-b]thiazole hydrochloride,
6-Adamantan-1-yl-3-(2,4-dimethoxy-pyrimidin-5-yl)-imidazo[2,1-b]thiazole hydrochloride,
8-[3-(3-Methoxyphenyl)-imidazo[2,1-b]thiazol-6-yl]-8-aza-spiro[4.5]decane hydrochloride,
8-[3-(3-Methoxyphenyl)-imidazo[2,1-b]thiazol-6-yl]-1,4-dioxa-8-aza-spiro[4.5]decane hydrochloride,
8-{1-[3-(3-Methoxyphenyl)-imidazo[2,1-b]thiazol-6-yl]-1-methyl-ethyl}-8-aza-spiro[4.5]decane hydrochloride,
8-{1-[3-(3-Methoxy-phenyl)-imidazo[2,1-b]thiazol-6-yl]-1-methyl-ethyl}-1,4-dioxa-8-aza-spiro[4.5]decane hydrochloride,
6-Adamantan-1-yl-3-(3,4-difluorophenyl)-imidazo[2,1-b]thiazole hydrochloride,
3-(6-tert-Butyl-imidazo[2,1-b]thiazole-3-yl)benzoic acid methyl ester hydrochloride,
Acetic acid, 3-(6-Adamantan-1-yl-imidazo[2,1-b]thiazol-3-yl)benzyl ester hydrochloride,
2-(6-Adamantan-1-yl-imidazo[2,1-b]thiazol-3-yl)phenylamine hydrochloride,
N[3-(6-Adamantan-1-yl-imidazo[2,1-b]thiazol-3-yl)-phenyl]-methanusulfonamide hydrochloride,
3-(6-Adamantan-1-yl-imidazo[2,1-b]thiazol-3-yl)-benzonitrile hydrochloride,
Acetic acid, 3-acetoxy-4-(6-Adamantan-1-yl-imidazo[2,1-b]thiazol-3-yl)phenyl ester hydrochloride,
Acetic acid, 2-acetoxy-4-(6-Adamantan-1-yl-imidazo[2,1-b]thiazol-3-yl)phenyl ester hydrochloride,
4-(6-Adamantan-1-yl-imidazo[2,1-b]thiazol-3-yl)benzene-1,2-diol hydrochloride,
Acetic acid, 4-acetoxy-3-(6-Adamantan-1-yl-imidazo[2,1-b]thiazol-3-yl)phenyl ester hydrochloride,
2-(6-Adamantan-1-yl-imidazo[2,1-b]thiazol-3-yl)benzene-1,4-diol hydrochloride,
6-Adamantan-1-yl-3-pyridin-2-yl-imidazo[2,1-b]thiazole dihydrochloride,
6-Adamantan-1-yl-3-(4-amino-3-methoxyphenyl)imidazo[2,1-b]thiazole hydrochloride,
6-Adamantan-1-yl-3-(3-chlorophenyl)-imidazo[2,1-b]thiazole hydrochloride,
3-(6-Adamantan-1-yl-imidazo[2,1-b]thiazol-3-yl)benzoic acid methyl ester hydrochloride,
6-Adamantan-1-yl-3-(3,5-dimethoxyphenyl)-imidazo[2,1-b]thiazole hydrochloride,
N-[5-(6-Adamantan-1-yl-imidazo[2,1-b]thiazol-3-yl)-2-methoxy-phenyl]-acetamide hydrochloride,
6-Adamantan-1-yl-2-phenyl-imidazo[2,1-b]thiazole hydrochloride,

and optical isomers, polymorphs and pharmaceutically-acceptable acid and base addition salts, hydrates, and solvates thereof.

### DETAILED DESCRIPTION OF THE INVENTION

For the purpose of the present invention, the carbon atom content of various hydrocarbon-contaihing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, (C₁₋₃)alkyl refers to alkyl of one to three carbon atoms, inclusive, (i.e., methyl, ethyl, propyl, and isopropyl), straight and branched forms thereof.

As used herein and as far as it is not defined in different manner elsewhere in this description or the accompanied claims, the term “C₁₋₆alkyl” represents straight or branched chain alkyl groups having 1, 2, 3, 4, 5 or 6 carbon atoms, examples of such alkyl groups include methyl, ethyl, n-propyl, 2-propyl, n-butyl, 2-butyl, iso-butyl, tert-butyl, n-pentyl, 2-pentyl, 3-pentyl, iso-pentyl, 2-methylbutyl, tert-amyl, n-hexyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl, and 3-ethylbutyl; the term “C₂₋₆alkenyl” represents straight or branched chain alkenyl groups having 2, 3, 4, 5 or 6 carbon atoms; the term “cycloC₃₋₁₂alkyl” represents monocyclic or bicyclic, or tricyclic alkyl groups having 3, 4, 5, 6, 7, 8, 9, 10, 11 or 12 carbon atoms, including cyclopropyl, cyclobutyl, cyclopentyl, cyclohexyl, bicyclo[2.2.1]heptyl and adamantanyl, wherein the “cycloC₃₋₁₂alkyl”-ring is optionally substituted by one or more (e.g., 1, 2, 3, or 4) substituents, which may be the same or different, selected independently from halogen, trifluoromethyl, trifluromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, di-C₁₋₆alkylamino, C₁₋₆alkylcarbonylamino, and C₁₋₆alkylenedioxy; the term “aryl” represents phenyl or naphthyl, wherein the phenyl or naphthyl group is optionally substituted by one or more (e.g., 1, 2, 3, or 4) substituents, which may be the same or different, selected independently from halogen, trifluoromethyl, trifluromethoxy, C₁₋₆alkyl, hydroxyC₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-C₁₋₆alkylamino, C₁₋₆alkylcarbonylamino, C₁₋₆alkylsulfonylamino, pyrrolidinyl, piperidinyl, morpholinyl, and piperazinyl or optionally substituted by C₁₋₆alkylenedioxy; the term C₁₋₆alkylenedioxy represents a straight or branched alkylenedioxy group having 1, 2, 3, 4, 5 or 6 carbon atoms, examples of such alkylenedioxy groups include —O—CH₂—O—, —O—(CH₂)₂—O— and —O—C(CH₃)₂—O—; the term “biaryl” represents biphenylene, including 4,4′-biphenylene, wherein one or both phenyl rings may optionally be substituted independently by one or more (e.g., 1, 2, 3, or 4) of the substituents independently selected from halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, pyrrolidinyl, piperidinyl, morpholinyl, and pyridinyl; the term “heteroaryl” represents an aromatic 5-6 membered ring comprising one to four heteroatoms selected from oxygen, sulfur and nitrogen or a bicyclic ring system having one 5-6 membered ring comprising one to four heteroatoms selected from oxygen, sulfur and nitrogen fused with a benzene ring or a 5-6 membered ring comprising one to four heteroatoms selected from oxygen, sulfur and nitrogen, wherein the heteroaryl is optionally substituted by one or more (e.g., 1, 2, 3, or 4) substituents, which may be the same or different, selected independently from halogen, trifluoromethyl, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkylcarbonyloxy, C₁₋₆alkylamino, di-C₁₋₆alkylamino, pyrrolidinyl, piperidinyl, morpholinyl, pyridinyl, and aryl; examples of such heteroaryl groups include furyl, thiophenyl, pyrrolyl, oxazolyl, isoxazolyl, thiazolyl, imidazolyl, oxadiazolyl, tetrazolyl, pyridinyl, pyrimidyl, benzofuryl, benzothiophenyl, indolyl, benzimidazolyl, benzoxazolyl, benzothiazolyl, quinolyl and isoquinolyl; the term “heterocyclyl” represents a saturated or unsaturated non-aromatic 3 to 12 membered ring comprising one to four heteroatoms selected from oxygen, sulfur and nitrogen or a saturated or unsaturated non-aromatic bicyclic ring system having 3 to 12 members comprising one to six heteroatoms selected from oxygen, sulfur and nitrogen, wherein the heterocyclic ring or ring system is optionally substituted by one or more (e.g., 1, 2, 3, or 4) substituents, which may be the same or different, selected independently from a halogen, trifluoromethyl, C₁₋₆alkyl, C₂₋₆alkenyl, C₁₋₆alkoxy, amino, hydroxy, nitro, cyano, C₁₋₆alkoxycarbonyl, C₁₋₆alkylamino, di-C₁₋₆alkylamino, pyrrolidinyl, piperidinyl, morpholinyl, pyridinyl, and aryl; examples of such heterocyclyl groups include piperidinyl, morpholinyl or piperazinyl; and the term “halogen” represents fluorine, chlorine, bromine and iodine.

The compounds of the present invention are named according to the IUPAC or CAS nomenclature system. Abbreviations which are well known to one of ordinary skill in the art may be used (e.g. “Ph” for phenyl, “Me” for methyl, “Et” for ethyl, “h” for hour or hours, and “rt” for room temperature).

Memantine, also known as 1-amino-3,5-dimethyladamantane, is disclosed, U.S. Pat. Nos. 4,122,193; 4,273,774; and 5,061,703, the subject matter of which patents is hereby incorporated by reference.

Neramexane, also known as 1-amino-1,3,3,5,5-pentamethylcyclohexane, is disclosed in detail in U.S. Pat. Nos. 6,034,134 and 6,071,966, the subject matter of which patents is hereby incorporated by reference.

Memantine and neramexane are systemically-active noncompetitive NMDA receptor antagonists having moderate affinity for the receptor. They exhibit strong voltage dependent characteristics and fast blocking/unblocking kinetics (Parsons et al., 1999, supra; Görtelmeyer et al., Arzneim-Forsch/Drug Res., 1992, 42:904-913; Winblad et al., Int. J. Geriat. Psychiatry, 1999, 14:135-146; Rogawski, Amino Acids, 2000, 19: 133-49; Danysz et al., Curr. Pharm. Des., 2002, 8:835-43; Jirgensons et. al., Eur. J. Med. Chem., 2000, 35: 555-565).

The term “analog” or “derivative” is used herein in the conventional pharmaceutical sense, to refer to a molecule that structurally resembles a reference molecule (such as imidazothiazole), but has been modified in a targeted and controlled manner to replace one or more specific substituents of the reference molecule with an alternate substituent, thereby generating a molecule which is structurally similar to the reference molecule. Synthesis and screening of analogs (e.g., using structural and/or biochemical analysis), to identify slightly modified versions of a known compound which may have improved or biased traits (such as higher potency and/or selectivity at a specific targeted receptor type, greater ability to penetrate mammalian blood-brain barriers, fewer side effects, etc.) is a drug design approach that is well known in pharmaceutical chemistry.

In addition, using methods known to those skilled in the art, analogs and derivatives of the compounds of the invention can be created which have improved therapeutic efficacy in controlling CNS diseases, i.e., higher potency and/or selectivity at a specific targeted receptor type, either greater or lower ability to penetrate mammalian blood-brain barriers (e.g., either higher or lower blood-brain barrier permeation rate), fewer side effects, etc.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

Compounds of the present invention may be in the form of pharmaceutically acceptable salts. ”Pharmaceutically acceptable salts” refers to those salts which possess the biological effectiveness and properties of the parent compound and which are not biologically or otherwise undesirable. The nature of the salt or isomer is not critical, provided that it is non-toxic and does not substantially interfere with the desired pharmacological activity.

It will be appreciated by those skilled in the art that compounds of the invention having a chiral center may exist in and be isolated in optically active and racemic forms. Some compounds may exhibit polymorphism. It is to be understood that the present invention ecompasses any racemic, optically-active, polymorphic, tautomeric, or stereoisomeric form, or mixture thereof, of a compound of the invention, which possesses the useful properties described herein.

The following Schemes describe the preparation of compounds of Formula I of the present invention. All of the starting materials are prepared by procedures described in these schemes, by procedures well known to one of ordinary skill in organic chemistry or can be obtained commercially. All of the final compounds of the present invention are prepared by procedures described in these charts or by procedures analogous thereto, which procedures would be well known to one of ordinary skill in organic chemistry. All of the variables used in the schemes are as defined below or as in the claims.

A compound of one embodiment of Formula I is prepared by condensation of 2-aminothiazole derivative 1 with an appropriate α-bromoketone 2 (Scheme 1).

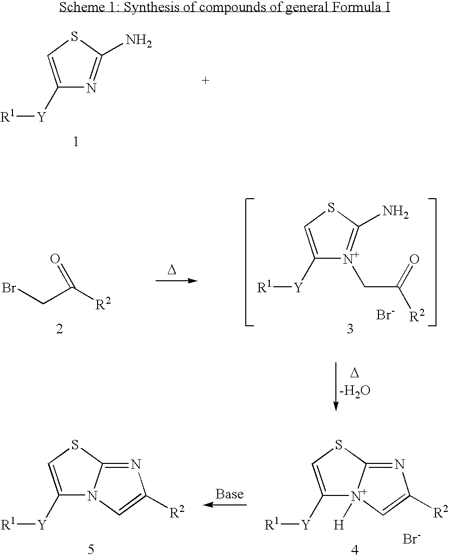

The condensation may conveniently be effected by refluxing a solution of reactants in an alcohol (e.g. ethanol, isopropanol or n-butanol). The reaction proceeds via a ring-alkylated intermediate 3, which upon cyclization and dehydration is converted into a hydrobromide salt of imidazo[2,1-b]thiazole 4. A free base 5 may be liberated from this salt by treatment with a base, such as a potassium carbonate solution. The free base may be further converted into a hydrochloride or any other pharmaceutically acceptable salt according to known procedures. 4-Substituted 2-aminothiazoles 1 are synthesized from corresponding α-bromoketones by condensation with thiourea according to well-documented literature precedents.

Alternatively, (Scheme 2) a compound of Formula I is prepared via alkylation of an imidazolinethione derivative 6 with an appropriate α-bromoketone 7 to give an intermediate 8 which then either cyclizes spontaneously, or is cyclized in the presence of polyphosphoric acid into an imidazo[2,1-b]thiazole derivative 5.

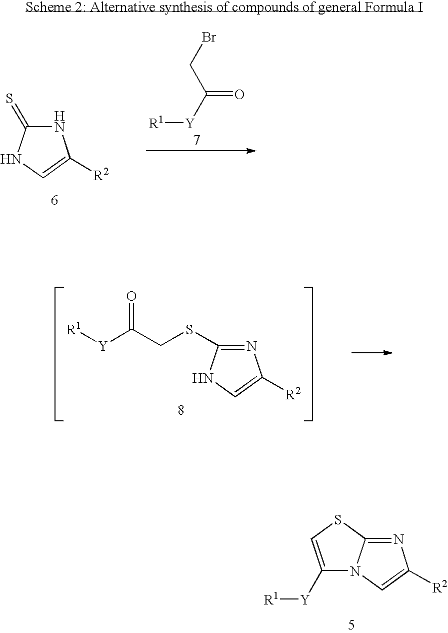

The reaction may conveniently be carried out by refluxing a solution of reactants in ethanol to provide a hydrobromide salt of imidazo[2,1-b]thiazole, from which a free base 5 is liberated by treatment with a base, such as potassium carbonate solution. The free base may be further converted into a hydrochloride or any other pharmaceutically acceptable salt according to known procedures.

Imidazolinethione derivatives 6 are conveniently prepared by a 2-step procedure, outlined in Scheme 3.

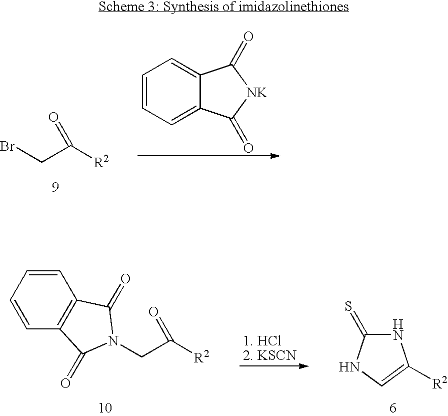

An α-bromoketone 9 is treated with potassium phthalimide to give an allkylated phthalimide derivative 10. Hydrolysis of compound 10 in refluxing aqueous hydrochloric acid yields an intermediate α-aminoketone which is treated with potassium thiocyanate in situ to yield an imidazolinethione 6.

Additional imidazo[2.1b]thiazoles of general Formula I may be prepared according to a novel strategy Scheme 4. This approach employs Suzuki coupling as a key step.

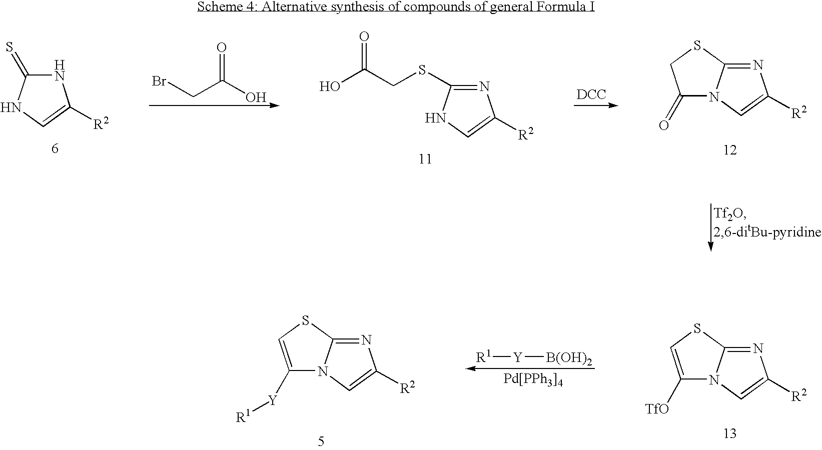

Imidazolinethione 6 is alkylated with bromoacetic acid to give a thioacid 11, which is cyclized into a lactam 12 by treatment with DCC. Lactam 12 is converted to an enol triflate 13 via reaction with triflic anhydride in the presence of 2,6-di-tert-butylpyridine as a base. Enol triflate 13 may be used in Suzuki coupling reactions with a variety of boronic acids or esters to provide 3,6-disubstituted imidazo[2,1-b]thiazoles 5. The free bases thus prepared may be further converted into hydrochlorides or other pharmaceutically acceptable salts according to known procedures. Given the high compatibility of Suzuki coupling with different functional groups, this approach allows for the preparation of a wide variety of compounds of general Formula I.

It will be apparent to those skilled in the art that the described synthetic procedures are merely representative in nature and that alternative synthetic processes are known to one of ordinary skill in organic chemistry.

### Experimental Part

The compounds and their preparation of the present invention will be better understood in connection with the following examples, which are intended as an illustration of and not a limitation upon the scope of the invention.

Hereinafter, “DMF” is defined as N,N-dimethylformamide, “HCI” as hydrochloric acid, “DMSO” as dimethylsulfoxide and “TMS” as tetramethylsilane.

### Synthesis of imidazo[2,1-b]thiazoles

### General Procedure 1

A mixture of a 2-aminothiazole (1 mmol) and an α-bromoketone (1.5 mmol) is refluxed in isopropanol (5 mL) for 12 h.

### Method A

The precipitate formed upon cooling is filtered, washed with isopropanol and dried to give sufficiently pure title compound as a hydrobromide salt.

### Method B

The mixture is evaporated in vacuo to dryness and the residue is partitioned between ethyl acetate and potassium carbonate solution. The organic phase is washed with water and brine, dried over anhydrous sodium sulfate and concentrated in vacuo. Purification of the residue by flash column chromatography provides the title compound as a free base. A portion (ca. 20 mg) of free base is dissolved in 2 mL of diethylether and treated with excess of an ethereal hydrogen chloride solution. The precipitate is filtered, washed with ether and dried to give the title compound as a hydrochloride salt.

### EXAMPLE 1

### 6-Adamantan-1-yl-3-(2,5-dimethylphenyl)-imidazo[2,1-b]thiazole hydrochloride

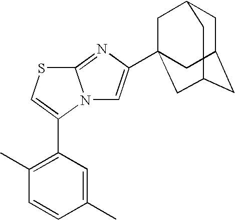

The title compound is synthesized according to General Procedure 1, Method B by reacting 2-amino-4-(2,5-dimethylphenyl)thiazole with 1-bromoacetyladamantane.

Physical characteristics are as follows:

Mp 158-160° C.; ¹H NMR (DMSO-d₆, TMS) δ: 1.73, 1.93, 2.02, 2.19, 2.34, 6.51, 6.81, 7.20

### EXAMPLE 2

### 6-Adamantan-1-yl-3-(2,5-difluorophenyl)-imidazo[2,1-b]thiazole hydrochloride

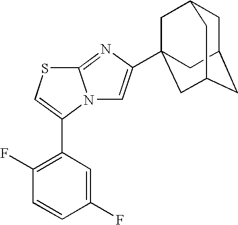

The title compound is synthesized according to General Procedure 1, Method B by reacting 2-amino-4-(2,5-difluorophenyl)thiazole with 1-bromoacetyladamantane.

Physical characteristics are as follows:

Mp 175-178° C.; ¹H NMR (DMSO-d₆, TMS) δ: 1.71, 1.92, 2.03, 6.76, 7.16, 7.22-7.36, 7.71.

### EXAMPLE 3

### 4-(6-Adamantan-1-yl-imidazo[2,1-b]thiazol-3-yl)-benzonitrile hydrochloride

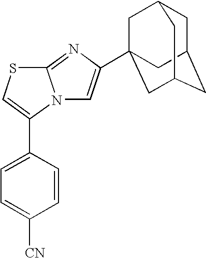

The title compound is synthesized according to General Procedure 1, Method B by reacting 2-amino-4-(4-cyanophenyl)thiazole with 1-bromoacetyladamantane.

Physical characteristics are as follows:

Mp 142-144° C.; ¹H NMR (DMSO-d₆, TMS) δ: 1.71, 1.92, 2.03, 6.86, 7.49, 7.65, 7.92.

### EXAMPLE 4

### 6-Adamantan-1-yl-3-(2,5-dimethoxyphenyl)-imidazo[2,1-b]thiazole hydrochloride

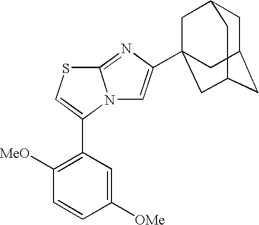

The title compound is synthesized according to General Procedure 1, Method B by reacting 2-amino-4-(2,5-dimethoxyphenyl)thiazole with 1-bromoacetyladamantane.

Physical characteristics are as follows:

Mp 133-135° C.; ¹H NMR (DMSO-d₆, TMS) δ: 1.71, 1.91, 2.03, 3.75, 3.77, 7.08, 7.19, 7.57, 7.66.

### EXAMPLE 5

### 6-Adamantan-1-yl-3-(4-methylphenyl)-imidazo[2,1-b]thiazole hydrochloride

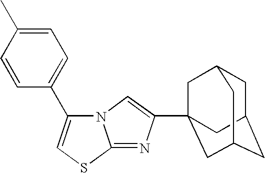

The title compound is synthesized according to General Procedure 1, Method B by reacting 2-amino-4-(4-methylphenyl)thiazole with 1-bromoacetyladamantane.

Physical characteristics are as follows:

Mp 244-247° C.; ¹H NMR (DMSO-d₆, TMS) δ: 1.71, 1.92, 2.03, 2.38, 7.38, 7.54, 7.65, 7.76.

### EXAMPLE 6

### 6-Adamantan-1-yl-3-(2,4-dimethylphenyl)-imidazo[2,1-b]thiazole hydrochloride

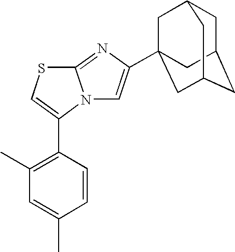

The title compound is synthesized according to General Procedure 1, Method B by reacting 2-amino-4-(2,4-dimethylphenyl)thiazole with 1-bromoacetyladamantane.

Physical characteristics are as follows:

Mp 227-230° C.; ¹H NMR (CDCl₃, TMS) (free base) δ: 1.75, 1.94, 2.03, 2.22, 2.39, 6.53, 6.83, 7.11, 7.16, 7.27.

### EXAMPLE 7

### 6-Adamantan-1-yl-3-(3,4-dimethoxyphenyl)-imidazo[2,1-b]thiazole hydrochloride

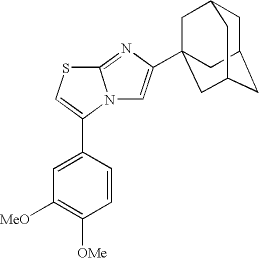

The title compound is synthesized according to General Procedure 1, Method B by reacting 2-amino-4-(3,4-dimethoxyphenyl)thiazole with 1-bromoacetyladamantane.

Physical characteristics are as follows:

Mp 196-197° C.; ¹H NMR (DMSO-d₆, TMS) δ: 1.72, 1.95, 2.03, 3.82, 3.84, 7.12, 7.27, 7.34, 7.58, 7.85.

### EXAMPLE 8

### 6-Adamantan-1-yl-3-(2,4-dimethoxyphenyl)-imidazo[2,1-b]thiazole hydrochloride

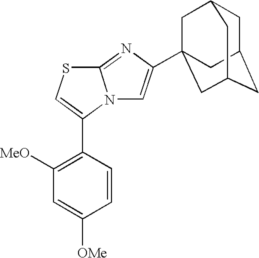

The title compound is synthesized according to General Procedure 1, Method B by reacting 2-amino-4-(2,4-dimethoxyphenyl)thiazole with 1-bromoacetyladamantane.

Physical characteristics are as follows:

Mp 191-192° C.; ¹H NMR (DMSO-d₆, TMS) δ: 1.71, 1.90, 2.03, 3.81, 3.84, 6.85, 6.70, 7.42, 7.46, 7.49.

### EXAMPLE 9

### 6-Adamantan-1-yl-3-benzofuran-2-yl-imidazo[2,1-b]thiazole hydrochloride

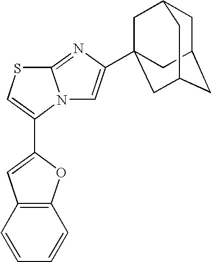

The title compound is synthesized according to General Procedure 1, Method B by reacting 2-amino-4-(benzofuran-2-yl)thiazole with 1-bromoacetyladamantane.

Physical characteristics are as follows:

Mp 310-313° C.; ¹H NMR (DMSO-d₆, TMS) δ: 1.74, 1.98, 2.05, 6.85, 6.70, 7.41, 7.73, 7.87, 8.08.

### EXAMPLE 10

### 6-Adamantan-1-yl-3-(4-fluorophenyl)-imidazo[2,1-b]thiazole hydrochloride

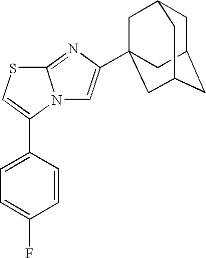

The title compound is synthesized according to General Procedure 1, Method B by reacting 2-amino-4-(4-fluorophenyl)thiazole with 1-bromoacetyladamantane.

Physical characteristics are as follows:

Mp 150-153° C.; ¹H NMR (DMSO-d₆, TMS) δ: 1.72, 1.93, 2.03, 6.84, 7.37-7.46, 7.64, 7.79-7.86.

### EXAMPLE 11

### 6-Adamantan-1-yl-3-benzo[1,3]dioxol-5-yl-imidazo[2,1-b]thiazole hydrochloride

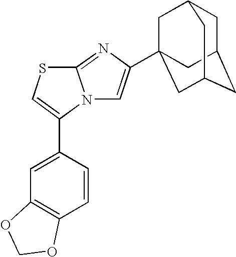

The title compound is synthesized according to General Procedure 1, Method B by reacting 2-amino-4-(benzo[1,3]dioxol-5-yl)thiazole with 1-bromoacetyladamantane.

Physical characteristics are as follows:

Mp 172-175° C.; ¹H NMR (Free base, CDCl₃, TMS) δ: 1.77, 1.99, 2.07, 6.06, 6.61, 6.93, 7.07, 7.11, 7.25.

### EXAMPLE 12

### 6-Adamantan-1-yl-3-thiophen-2-yl-imidazo[2,1-b]thiazole hydrobromide

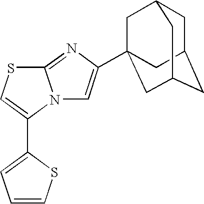

The title compound is synthesized according to General Procedure 1, Method A by reacting 2-amino-4-(thiophen-2-yl)thiazole with 1-bromoacetyladamantane.

Physical characteristics are as follows:

Mp>135° C. (decomp.); ¹H NMR (DMSO-d₆, TMS) δ: 1.72, 1.93, 2.03, 7.28, 7.67, 7.78, 7.80, and 7.92.

### EXAMPLE 13

### 6-Adamantan-1-yl-3-(4-methoxy-3-methyl-phenyl)-imidazo[2,1-b]thiazole hydrochloride

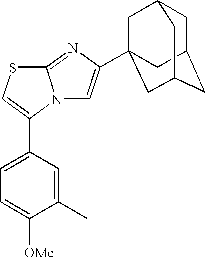

The title compound is synthesized according to General Procedure 1, Method B by reacting 2-amino-4-(4-methoxy-3-methylphenyl)thiazole with 1-bromoacetyladamantane.

Physical characteristics are as follows:

Mp 179-181° C.; ¹H NMR (DMSO-d₆, TMS) δ: 1.72, 1.95, 2.04, 2.23, 3.86, 7.11, 7.52, 7.58, and 7.81.

### EXAMPLE 14

### 6-Adamantan-1-yl-3-(4-methoxyphenyl)-imidazo[2,1-b]thiazole hydrochloride

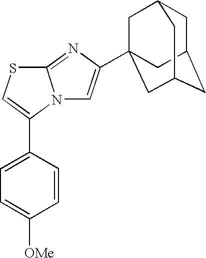

The title compound is synthesized according to General Procedure 1, Method B by reacting 2-amino-4-(4-methoxyphenyl)thiazole with 1-bromoacetyladamantane.

Physical characteristics are as follows:

Mp 310-313° C.; ¹H NMR (DMSO-d₆, TMS) δ: 1.74, 1.95, 2.05, 3.85, 7.14, 7.51, 7.72, 7.78.

### EXAMPLE 15

### 6-Adamantan-1-yl-3-(3-trifluoromethoxyphenyl)-imidazo[2,1-b]thiazole hydrochloride

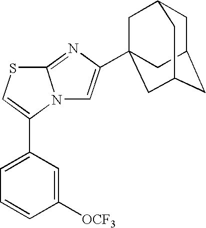

The title compound is synthesized according to General Procedure 1, Method B by reacting 2-aamino-4-(3-trifluoromethoxyphenyl)thiazole with 1-bromoacetyladamantane.

Physical characteristics are as follows:

Mp 189-201° C.; ¹H NMR (CDCl₃, TMS) δ: 1.80, 2.07, 2.12, 7.34, 7.43, 7.46, 7.56-7.70.

### EXAMPLE 16

### 6-Adamantan-1-yl-3-(2,5-dimethylthiophen-3-yl)-imidazo[2,1-b]thiazole hydrochloride

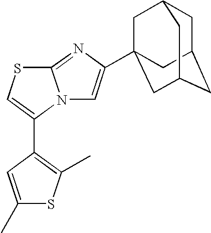

The title compound is synthesized according to General Procedure 1, Method B by reacting 2-amino-4-(2,5-dimethylthiophen-3-yl)thiazole with 1-bromoacetyladamantane.

Physical characteristics are as follows:

Mp 206-209° C.; ¹H NMR (free base, CDCl₃, TMS) δ: 1.76, 1.97, 2.06, 2.43, 2.47, 6.47, 6.78, 7.04.

### EXAMPLE 17

### 6-Adamantan-1-yl-3-(3-trifluoromethylphenyl)-imidazo[2,1-b]thiazole hydrochloride

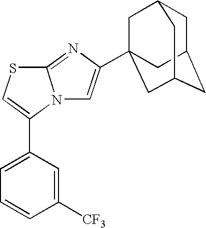

The title compound is synthesized according to General Procedure 1, Method B by reacting 2-amino-4-(3-trifluoromethylphenyl)thiazole with 1-bromoacetyladamantane.

Physical characteristics are as follows:

Mp 211-213° C.; ¹H NMR (CDCl₃, TMS) δ: 1.79, 2.07, 2.12, 7.22, 7.43, 7.76-7.83, 7.87.

### EXAMPLE 18

### 6-(Adamantan-1-yl)-3-(2,4,6-trimethylphenyl)-imidazo[2,1-b]thiazole hydrochloride

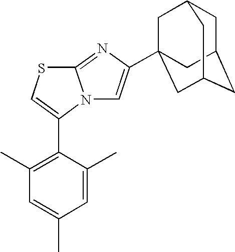

The title compound is synthesized according to General Procedure 1, Method B by reacting 2-amino-4-(2,4,6-trimethylphenyl)thiazole with 1-bromoacetyladamantane.

Physical characteristics are as follows:

Mp 163-164° C.; ¹H NMR (CDCl₃, TMS) δ: 1.74, 1.92, 2.03, 2.09, 2.35, 6.47, 6.64, 6.98.

### EXAMPLE 19

### 6-Adamantan-1-yl-3-(2-trifluoromethylphenyl)-imidazo[2,1-b]thiazole hydrochloride

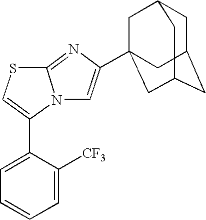

The title compound is synthesized according to General Procedure 1, Method B by reacting 2-amino-4-(2-trifluoromethylphenyl)thiazole with 1-bromoacetyladamantane.

Physical characteristics are as follows:

Mp 175-176° C.; ¹H NMR (DMSO-d₆) δ: 1.71, 1.92, 2.02, 7.53, 7.66, 7.80, 7.85-7.95, 8.02.

### EXAMPLE 20

### 6-Adamantan-1-yl-3-(2,5-diethylphenyl)-imidazo[2,1-b]thiazole hydrochloride

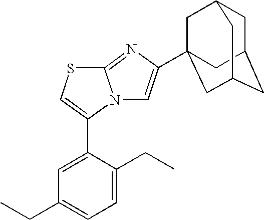

The title compound is synthesized according to General Procedure 1, Method B by reacting 2-amino-4-(2,5-diethylphenyl)thiazole with 1-bromoacetyladamantane.

Physical characteristics are as follows:

Mp 103-106° C.; ¹H NMR (DMSO-d₆, TMS) δ: 1.02, 1.17, 1.68, 1.89, 1.99, 2.44, 2.62, 7.24, 7.35, 7.37, 7.53.

### EXAMPLE 21

### 6-Adamantan-1-yl-3-(1-methyl-1H-pyrrol-2-yl)-imidazo[2,1-b]thiazole hydrochloride

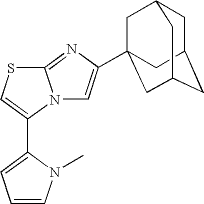

The title compound is synthesized according to General Procedure 1, Method B by reacting 2-amino-4-(1-methyl-1H-pyrrol-2-yl)thiazole with 1-bromoacetyladamantane.

Physical characteristics are as follows:

Mp 207-210° C.; ¹H NMR (DMSO-d₆, TMS) δ: 1.72, 1.94, 2.03, 3.69, 6.22, 6.64, 7.08, 7.53, 7.64.

### EXAMPLE 22

### 6-Adamantan-1-yl-3-(1,2,5-trimethyl-1H-pyrrol-3-yl)-imidazo[2,1-b]thiazole hydrochloride

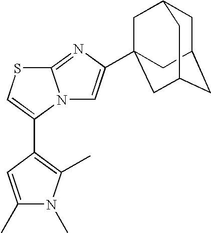

The title compound is synthesized according to General Procedure 1, Method B by reacting 2-amino-4-(1,2,3-trimethyl-1H-pyrrol-3-yl)thiazole with 1-bromoacetyladamantane.

Physical characteristics are as follows:

Mp 169-172° C.; ¹H NMR (Free base, CDCl₃, TMS) δ: 1.77, 1.99, 2.04, 2.28, 3.47, 6.08, 6.32, 7.17.

### EXAMPLE 23

### 6-Cyclohexyl-3-(2,5-dimethoxyphenyl)-imidazo[2,1-b]thiazole hydrochloride

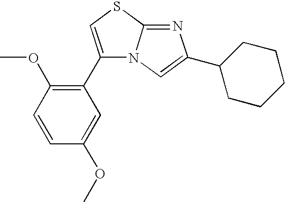

The title compound is synthesized according to General Procedure 1, Method B by reacting 2-amino-4-(2,5-dimethylphenyl)thiazole with 1-bromoacetylcyclohexane.

Physical characteristics are as follows:

Mp 158-160° C.; ¹H NMR (free base, CDCl₃, TMS) δ: 1.27, 1.68-1.81, 2.04-2.09, 2.65, 3.78, 3.81, 6.74, 6.96, 7.03, 7.05.

### EXAMPLE 24

### 6-Cyclohexyl-3-(2,5-difluorophenyl)-imidazo[2,1-b]thiazole hydrochloride

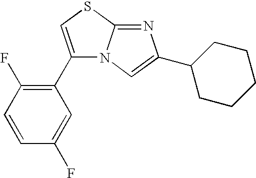

The title compound is synthesized according to General Procedure 1, Method B by reacting 2-amino-4-(2,5-difluorophenyl)thiazole with 1-bromoacetylcyclohexane.

Physical characteristics are as follows:

Mp 143-145° C.; ¹H NMR (free base, CDCl₃, TMS) δ: 1.36-1.47, 1.70-1.81, 2.05-2.10, 2.65, 6.89, 7.12-7.23, 7.31.

### EXAMPLE 25

### 3-(6-Adamantan-1-yl-imidazo[2,1-b]thiazol-3-yl)-5-methoxy-1,2-dimethyl-1H-indole hydrochloride

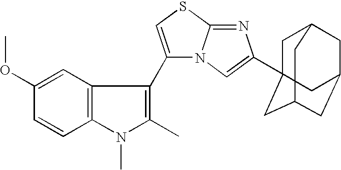

The title compound is synthesized according to General Procedure 1, Method B by reacting 3-(2-aminothiazol-4-yl)-5-methoxy-1,2-dimethyl-1H-indole with 1-bromoacetyladamantane.

Physical characteristics are as follows:

Mp 172-175° C.; ¹H NMR (Free base, CDCl₃, TMS) δ: 1.75, 1.95, 2.03, 2.44, 3.77, 3.73, 6.49, 6.83, 6.90, 6.97, 7.24.

### EXAMPLE 26

### 6-Adamantan-1-yl-3-(3-bromophenyl)-imidazo[2,1-b]thiazole

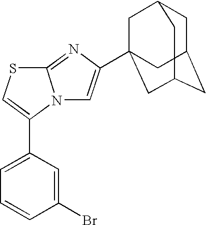

The title compound is synthesized according to General Procedure 1, Method B by reacting 2-amino-4-(3-bromophenyl)thiazole with 1-bromoacetyladamantane.

### EXAMPLE 27

### 6-Adamantan-1-yl-3-(3-acetylaminophenyl)-imidazo[2,1-b]thiazole

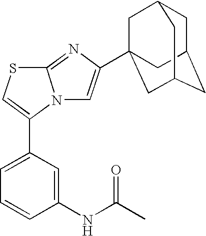

N,N′-dimethylethylenediamine (0.12 mmol) is added dropwise to a slurry of copper (I) iodide (0.1 mmol) in 1 mL of dioxane. The resulting clear solution is transferred via cannula to a solution of 6-adamantan-1-yl-3-(3-bromophenyl)-imidazo[2,1-b]thiazole (1 mmol), acetamide (1.5 mmol) and potassium phosphate (2 mmol) in 3 mL of dioxane. The mixture is heated to 110° C. for 12 h, then cooled and partitioned between ethyl acetate and water. The organic phase is washed with water, brine, dried over anhydrous sodium sulfate and concentrated in vacuo. Purification of the residue by flash column chromatography provides the title compound as a colorless crystalline solid.

Physical characteristics are as follows:

Mp 98-100° C.; ¹H NMR (CDCl₃, TMS) δ: 1.74, 1.96, 2.03, 2.21, 6.67, 7.33, 7.31, 7.41, 7.54, 7.92, 8.22.

### EXAMPLE 28

### 6-Adamantan-1-yl-3-(3-aminophenyl)-imidazo[2,1-b]thiazole hydrochloride

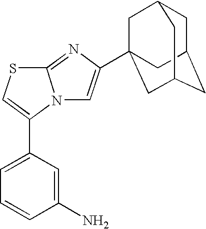

6-Adamantan-1-yl-3-(3-acetylaminophenyl)-imidazo[2,1-b]thiazole (0.5 mmol) is suspended in concentrated hydrochloric acid. The mixture is heated to 60° C. for 24 h, then cooled, neutralized with sodium bicarbonate solution and extracted with ethyl acetate. The organic phase is washed with water, brine, dried over anhydrous sodium sulfate and concentrated in vacuo. Purification of the residue by flash column chromatography provides the title compound as a free base. A portion (ca. 20 mg) of free base is dissolved in 2 mL of diethyl ether and treated with excess of ethereal hydrogen chloride solution. The precipitate is filtered, washed with ether and dried to give the title compound as a hydrochloride salt.

Physical characteristics are as follows:

Mp>180° C. (decomp.); ¹H NMR (free base, CDCl₃, TMS) δ: 1.77, 1.98, 2.06, 6.64, 6.76, 6.90, 6.99, 7.27, 7.29.

### EXAMPLE 29

### 3-(2,5-Dimethoxyphenyl)-6-(1-methyl-1-phenylethyl)-imidazo[2,1-b]thiazole

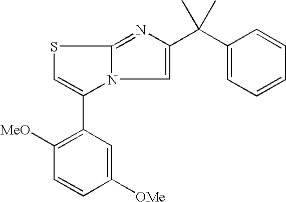

The title compound is prepared according to General Procedure 1.

### EXAMPLE 30

### 3-(2,5-Dimethylphenyl)-6-(1-methyl-1-phenylethyl)-imidazo[2,1-b]thiazole

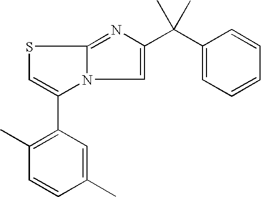

The title compound is prepared according to General Procedure 1

### EXAMPLE 31

### 3-(2,5-Dimethoxyphenyl)-6-piperdin-1-yl-imidazo[2,1-b]thiazole

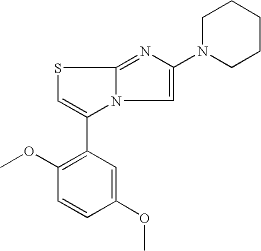

The title compound is prepared according to General Procedure 1.

### EXAMPLE 32

### 6-Azepan-1-yl-3-2,5-dimethoxyphenyl)-imidazo[2,1-b]thiazole

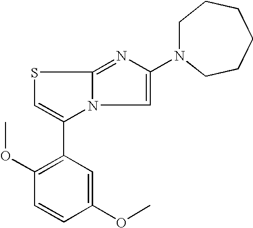

The title compound is prepared according to General Procedure 1.

### EXAMPLE 33

### 3-(2,5-Dimethoxyphenyl)-6-[1-(4-fluorophenyl)-1-methyl-ethyl]-imidazo[2,1-b]thiazole hydrochloride

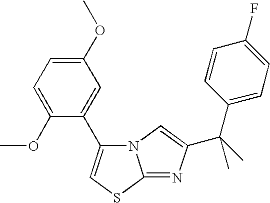

The title compound is synthesized according to General Procedure 1, Method B by reacting 2-amino-4-(2,5-dimethoxyphenyl)thiazole with 1-bromo-3-(4-fluorophenyl)-3-methyl-butan-2-one.

Physical characteristics are as follows:

Mp 155-157° C.; ¹H NMR (free base, CDCl₃, TMS) δ: 1.89, 3.75, 3.81, 6.80, 6.95-7.10, 7.28, 7.36-7.42.

### EXAMPLE 34

### 3-(2,4-Dimethoxyphenyl)-6-[1-(3-fluorophenyl)-1-methyl-ethyl]-imidazo[2,1-b]thiazole hydrochloride

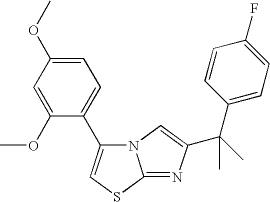

The title compound is synthesized according to General Procedure 1, Method B by reacting 2-amino-4-(2,4-dimethoxyphenyl)thiazole with 1-bromo-3-(3-fluorophenyl)-3-methyl-butan-2-one.

Physical characteristics are as follows:

Mp 122-126° C.; ¹H NMR (free base, CDCl₃, TMS) δ: 1.70, 3.80, 3.87, 6.56, 6.58, 6.64, 6.84, 6.95, 7.05, 7.10-7.28, 7.36.

### EXAMPLE 35

### 3-(2,4-Dimethoxyphenyl)-6-(1-methyl-1-phenyl-ethyl)-imidazo[2,1-b]thiazole hydrochloride

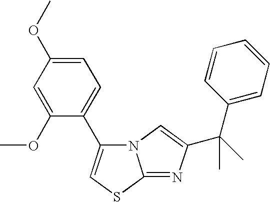

The title compound is synthesized according to General Procedure 1, Method B by reacting 2-amino-4-(2,4-dimethoxyphenyl)thiazole with 1-bromo-3-phenyl-3-methyl-butan-2-one.

Physical characteristics are as follows:

Mp 150-152° C.; ¹H NMR (free base, CDCl₃, TMS) δ: 1.72, 3.79, 3.86, 6.56, 6.58, 6.64, 6.95, 7.10-7.40.

### EXAMPLE 36

### 6-tert-Butyl-3-(2,4-dimethylphenyl)-imidazo[2,1-b]thiazole hydrochloride

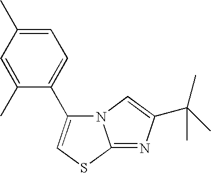

The title compound is synthesized according to General Procedure 1, Method B by reacting 2-amino-4-(2,4-dimethylphenyl)thiazole with 1-bromo-3,3-dimethyl-butan-2-one

Physical characteristics are as follows:

Mp 182-185° C.; ¹H NMR (DMSO-d₆, TMS) δ: 1.31, 2.22, 2.37, 7.20, 7.28, 7.38, 7.48, 7.52.

### EXAMPLE 37

### 6-tert-Butyl-3-(p-tolyl)-imidazo[2,1-b]thiazole hydrochloride

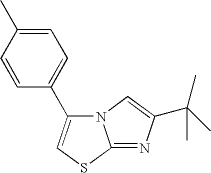

The title compound is synthesized according to General Procedure 1, Method B by reacting 2-amino-4-(4-methylphenyl)thiazole with 1-bromo-3,3-dimethyl-butan-2-one.

Physical characteristics are as follows:

Mp 190-192° C.; ¹H NMR (DMSO-d₆, TMS) δ: 1.34, 2.39, 7.40, 7.60, 7.67, 7.85.

### EXAMPLE 38

### 6-tert-Butyl-3-(3-methoxyphenyl)-imidazo[2,1-b]thiazole hydrochloride

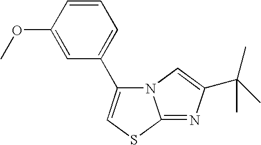

The title compound is synthesized according to General Procedure 1, Method B by reacting 2-amino-4-(3-methoxylphenyl)thiazole with 1-bromo-3,3-dimethyl-butan-2-one.

Physical characteristics are as follows:

Mp 162-165° C.; ¹H NMR (DMSO-d₆, TMS) δ: 1.34, 3.84, 7.13, 7.28-7.35, 7.51, 7.67, 7.87.

### EXAMPLE 39

### 6-tert-Butyl-3-(2,5-dimethoxyphenyl)-imidazo[2,1-b]thiazole hydrochloride

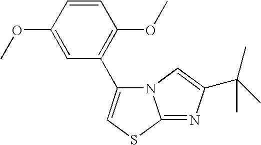

The title compound is synthesized according to General Procedure 1, Method B by reacting 2-amino-4-(2,5-dimethoxylphenyl)thiazole with 1-bromo-3,3-dimethyl-butan-2-one.

Physical characteristics are as follows:

Mp 169-171° C.; ¹H NMR (DMSO-d₆, TMS) δ: 1.33, 3.77, 3.79, 7.01, 7.10-7.24, 7.53, 7.60.

### EXAMPLE 40

### 3-(3-bromophenyl)-6-tert-butyl-imidazo[2,1-b]thiazole hydrochloride

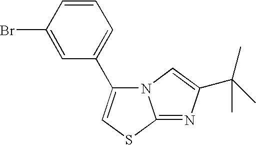

The title compound is synthesized according to General Procedure 1, Method B by reacting 2-amino-4-(3-bromophenyl)thiazole with 1-bromo-3,3-dimethyl-butan-2-one.

Physical characteristics are as follows:

Mp 188-190° C.; ¹H NMR (CDCl₃, TMS) δ: 1.35, 6.73, 7.29, 7.39, 7.56-7.63, 7.77.

### EXAMPLE 41

### 8-[3-(3-Methoxyphenyl)-imidazo[2,1-b]thiazol-6-yl]-8-aza-spiro[4.5]decane hydrochloride

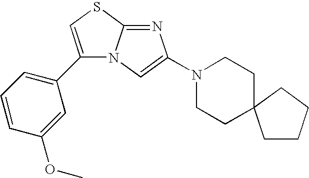

The title compound is synthesized according to General Procedure 1, Method B by reacting 2-amino-4-(3-methoxyphenyl)thiazole with 1-1-(8-Aza-spiro[4.5]dec-8-yl)-2-chloro-ethanone.

### EXAMPLE 42

### 8-[3-(3-Methoxyphenyl)-imidazo[2,1-b]thiazol-6-yl]-1,4-dioxa-8-aza-spiro[4.5]decane hydrochloride

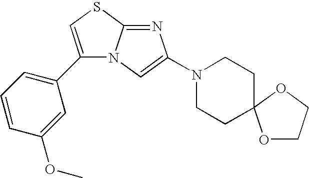

The title compound is synthesized according to General Procedure 1, Method B by reacting 2-amino-4-(3-methoxyphenyl)thiazole with 2-chloro-1-(1,4-dioxa-8-aza-spiro[4.5]dec-8-yl)-ethanone.

### EXAMPLE 43

### 8-{1-[3-(3-Methoxyphenyl)-imidazo[2,1-b]thiazol-6-yl]-1-methyl-ethyl}-8-aza-spiro[4.5]decane hydrochloride

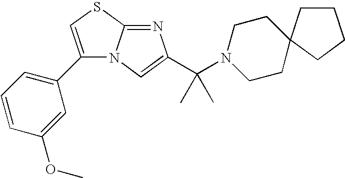

The title compound is synthesized according to General Procedure 1, Method B by reacting 2-amino-4-(3-methoxyphenyl)thiazole with 3-(8-aza-spiro[4.5]dec-8-yl)-1-bromo-3-methyl-butan-2-one.

### EXAMPLE 44

### 8-{1-[3-(3-Methoxy-phenyl)-imidazo[2,1-b]thiazol-6-yl]-1-methyl-ethyl}-1,4-dioxa-8-aza-spiro[4.5]decane hydrochloride

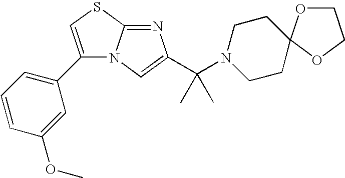

The title compound is synthesized according to General Procedure 1, Method B by reacting 2-amino-4-(3-methoxyphenyl)thiazole with 1-chloro-3-(1,4-dioxa-8-aza-spiro[4.5]dec-8-yl)-3-methyl-butan-2-one.

### Preparation 1

### Synthesis of 6-Adamantan-1-yl-imidazo[2,1-b]thiazol-3-one

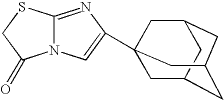

Bromoacetic acid (0.62 g, 4.50 mmol) is added to a solution of 4-adamantan-1-yl-1,3-dihydro-imidazole-2-thione (1 g, 4.27 mmol) in 10 mL of ethanol. The mixture is heated to reflux for 4 h, then cooled to room temperature. The precipitate is filtered, washed with cold ethanol and dried to give 1.14 g of (4-adamantan-1-yl-1H-imidazol-2-ylsulfanyl)-acetic acid as a white powder. This material is suspended in 40 mL of methylene chloride and DCC (0.96 g, 4.60 mmol) is added. The mixture is stirred at room temperature for 24 h; then diluted with aqueous sodium carbonate solution. The organic phase is separated, dried over anhydrous Na₂SO₄ and evaporated in vacuo. The residue is purified by flash column chromatography to provide the title compound as a crystalline solid.

Physical characteristics are as follows:

Mp 130-132° C.; ¹H NMR (CDCl₃, TMS) δ: 1.72, 1.86, 2.01, 4.24, 6.98.

### Preparation 2

### Synthesis of Trifluoromethanesulfonic acid 6-adamantan-1-yl-imidazo[2,1-b]thiazol-3-yl ester

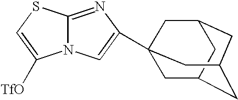

Triflic anhydride (158 μL, 0.94 mmol) is dropwise added to a solution of 6-adamantan-1-yl-imidazo[2,1-b]thiazol-3-one (129 mg, 0.47 mmol) and 2,6-di-^{t}butylpyridine (286 μL, 1.27 mmol) at −78° C. The mixture is stirred at that temperature for 1 h, then allowed to reach room temperature and then partitioned between aqueous sodium carbonate solution and ethyl acetate. The organic phase is separated, dried over anhydrous Na₂SO₄ and evaporated in vacuo. The residue is purified by flash column chromatography to provide the title compound as a colorless oil.

Physical characteristics are as follows:

¹H NMR (CDCl₃, TMS) δ: 1.73, 1.93, 2.03, 6.59, 7.08.

### General Procedure 2—Suzuki coupling reactions of trifluoromethanesulfonic acid 6-adamantan-1-yl-imidazo[2,1-b]thiazol-3-yl ester

A solution of 0.45 mmol of boronic acid or ester, 0.03 mmol of Pd[PPh₃]₄, and 0.25 mmol of trifluoromethanesulfonic acid 6-adamantan-1-yl-imidazo[2,1-b]thiazole-3-yl ester in 2 mL of THF is combined with 2 mL of 2M aqueous K₂CO₃ solution. The heterogenuous mixture is heated by microwaves at 120° C. for 20 min, then partitioned between aqueous sodium carbonate solution and ethyl acetate. The organic phase is separated, dried over anhydrous Na₂SO₄ and evaporated in vacuo. The residue is purified by flash column chromatography to provide the coupling product as a free base. A portion (ca 20 mg) of free base is dissolved in diethylether (2 mL) and and treated with excess ethereal hydrogen chloride solution. The precipitate is filtered, washed with ether and dried to give the title compound as hydrochloride salt.

### EXAMPLE 45

### 6-Adamantan-1-yl-3-(6-aminopyridin-3-yl)imidazo[2,1-b]thiazole hydrochloride

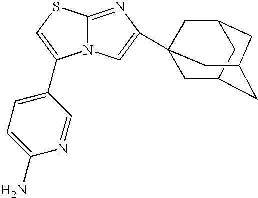

The title compound is synthesized according to General Procedure 2 by reacting trifluoromethanesulfonic acid 6-adamantan-1-yl-imidazo[2,1-b]thiazol-3-yl ester with 5-(4,4,5,5-tetramethyl-[1,3,2]dioxaborolan-2-yl)-pyridin-2-ylamine.

Physical characteristics are as follows:

Mp 226° C. (decomp.); ¹H NMR (free base, CDCl₃, TMS) δ: 1.75, 1.96, 2.04, 6.57, 6.60, 7.19, 7.64, 8.31.

### EXAMPLE 46

### 6-Adamantan-1-yl-3-pyridin-3-yl-imidazo[2,1-b]thiazole hydrochloride

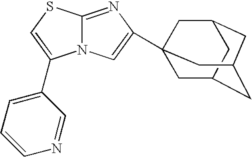

The title compound is synthesized according to General Procedure 2 by reacting trifluoromethanesulfonic acid 6-adamantan-1-yl-imidazo[2,1-b]thiazol-3-yl ester with 3-(4,4,5,5-tetramethyl-[1,3,2]dioxaborolan-2-yl)-pyridine.

Physical characteristics are as follows:

Mp 250-253° C.; ¹H NMR (free base, CDCl3, TMS) δ: 1.78, 1.98, 2.07, 6.81, 7.27, 7.46, 7.95, 8.71, 8.91.

### EXAMPLE 47

### 6-Adamantan-1-yl-3-(6-methoxy-pyridin-3-yl)-imidazo[2,1-b]thiazole hydrochloride

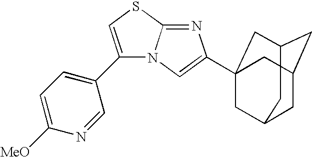

The title compound is synthesized according to General Procedure 2 by reacting trifluoromethanesulfonic acid 6-adamantan-1-yl-imidazo[2,1-b]thiazol-3-yl ester with 2-methoxy-5-pyridineboronic acid.

Physical characteristics are as follows:

Mp 145-148° C.; ¹H NMR (free base, CDCl3, TMS) δ: 1.77, 1.97, 2.04, 4.01, 6.66, 6.89, 7.22, 7.81, 8.45.

### EXAMPLE 48

### 6-Adamantan-1-yl-3-(2-fluoro-pyridin-3-yl)-imidazo[2,1-b]thiazole hydrochloride

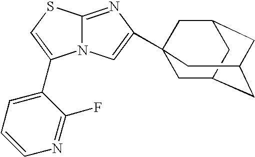

The title compound is synthesized according to General Procedure 2 by reacting trifluoromethanesulfonic acid 6-adamantan-1-yl-imidazo[2,1-b]thiazol-3-yl yl ester with 2-fluoro-3-pyridineboronic acid.

Physical characteristics are as follows:

Mp semisolid; ¹H NMR (free base, CDCl₃, TMS) δ: 1.77, 1.98, 2.08, 6.97, 7.15, 7.38, 8.07, 8.34.

### EXAMPLE 49

### 6-Adamantan-1-yl-3-pyridin-4-yl-imidazo[2,1-b]thiazole hydrochloride

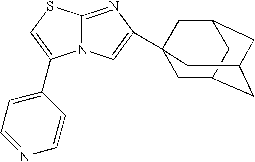

The title compound is synthesized according to General Procedure 2 by reacting trifluoromethanesulfonic acid 6-adamantan-1-yl-imidazo[2,1-b]thiazol-3-yl ester with 4-pyridineboronic acid.

Physical characteristics are as follows:

Mp 213-215° C.; ¹H NMR (DMSO-d₆, TMS) δ: 1.73, 1.97, 2.05, 8.06, 8.12, 8.24, 8.90.

### EXAMPLE 50

### 6-Adamantan-1-yl-3-(2,4-dimethoxy-pyrimidin-5-yl)-imidazo[2,1-b]thiazole hydrochloride

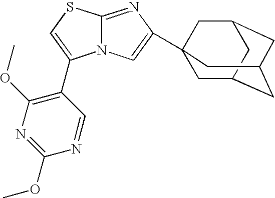

The title compound is synthesized according to General Procedure 2 by reacting trifluoromethanesulfonic acid 6-adamantan-1-yl-imidazo[2,1-b]thiazol-3-yl ester with 2,4-dimethoxy-5-(4,4,5,5-tetramethyl-[1,3,2]dioxaborolan-2-yl)-pyramidine.

Physical characteristics are as follows:

Mp 201-204° C.; ¹H NMR (free base, CDCl₃, TMS) δ: 1.76, 1.95, 2.05, 4.06, 4.07, 6.78, 6.99, 8.45.

### EXAMPLE 51

### 6-Adamantan-1-yl-3-(3-dimethylaminophenyl)-imidazo[2,1-b]thiazole hydrochloride

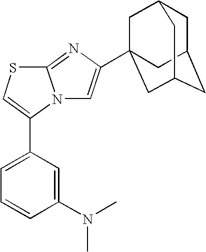

The title compound is synthesized according to General Procedure 2 by reacting trifluoromethanesulfonic acid 6-adamantan-1-yl-imidazo[2,1-b]thiazol-3-yl ester with 3-(4,4,5,5-tetramethyl-[1,3,2]dioxaborolan-2-yl)-N,N-dimethylaniline.

Physical characteristics are as follows:

Mp 136-140° C. (decomp.); ¹H NMR (DMSO-d₆, TMS) (free base) δ: 1.70, 1.96, 2.03, 3.02, 7.27-7.53, 7.49, 7.76, 7.91.

### EXAMPLE 52

### 4-(6-Adamantan-1-yl-imidazo[2,1-b]thiazole-3-yl)benzene-1,3-diol hydrochloride

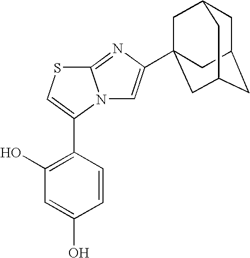

The title compound is synthesized according. to General Procedure 2 by reacting trifluoromethanesulfonic acid 6-adamantan-1-yl-imidazo[2,1-b]thiazol-3-yl ester with 4-(4,4,5,5-tetramethyl-[1,3,2]dioxaborolan-2-yl)-benzene-1,3-diol.

Physical characteristics are as follows:

Mp 248-250° C.; ¹H NMR (DMSO-d₆, TMS) (free base) δ: 1.72, 1.92, 2.04, 6.39, 6.50, 7.23, 7.38, 7.49.

### EXAMPLE 53

### [3-(6-Adamantan-1-yl-imidazo[2,1-b]thiazole-3-yl)phenyl]-methanol

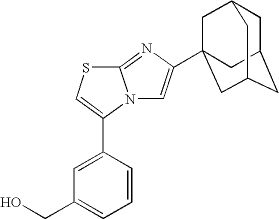

The title compound is synthesized according to General Procedure 2 by reacting trifluoromethanesulfonic acid 6-adamantan-1-yl-imidazo[2,1-b]thiazol-3-yl ester with 3-(4,4,5,5-tetramethyl-[1,3,2]dioxaborolan-2-yl)-benzyl alcohol.

Physical characteristics are as follows:

Mp 165° C. (decomp.); ¹H NMR (CDCl₃, TMS) δ: 1.7, 1.95, 2.04, 4.79, 6.68, 7.27, 7.44-7.55, 7.63.

### EXAMPLE 54

### 6-Adamantan-1-yl-3-(3-methoxyphenyl)-imidazo[2,1-b]thiazole hydrochloride

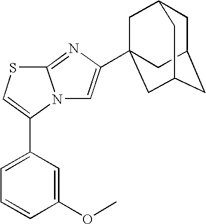

The title compound is synthesized according to General Procedure 2 by reacting trifluoromethanesulfonic acid 6-adamantan-1-yl-imidazo[2,1-b]thiazol-3-yl ester with 3-methoxybenzeneboronic acid.

Physical characteristics are as follows:

Mp 159-161° C.; ¹H NMR (DMSO-d₆, TMS) (free base) δ: 1.73, 1.96, 2.05, 3.85, 7.15, 7.27, 7.33, 7.51, 7.68, 7.83.

### EXAMPLE 55

### 3-(6-Adamantan-1-yl-imidazo[2,1-b]thiazole-3-yl)phenol hydrochloride

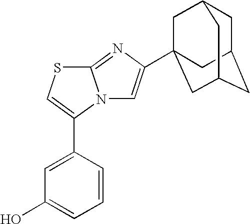

The title compound is synthesized according to General Procedure 2 by reacting trifluoromethanesulfonic acid 6-adamantan-1-yl-imidazo[2,1-b]thiazol-3-yl ester with 3-(4,4,5,5-tetramethyl-[1,3,2]dioxaborolan-2-yl)-phenol.

Physical characteristics are as follows:

Mp 188-190° C.; ¹H NMR (DMSO-d₆, TMS) (free base) δ: 1.72, 1.90, 2.01, 6.89, 7.11, 7.16, 7.31, 7.35, 7.48.

### EXAMPLE 56

### 6-Adamantan-1-yl-3-(3,4-difluorophenyl)-imidazo[2,1-b]thiazole hydrochloride

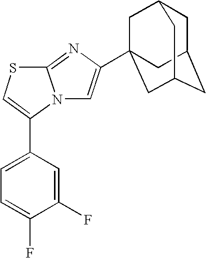

The title compound is synthesized according to General Procedure 2 by reacting trifluoromethanesulfonic acid 6-adamantan-1-yl-imidazo[2,1-b]thiazol-3-yl ester with 3,4-difluorobenzeneboronic acid.

Physical characteristics are as follows:

Mp 170-172° C.; ¹H NMR (DMSO-d₆, TMS) (free base) δ: 1.74, 1.98, 2.07, 7.62-7.70, 7.76, 7.88-7.98.

### EXAMPLE 57

### Acetic acid 3-(6-adamantan-1-yl-imidazo[2,1-b]thiazole-3-yl)phenylester

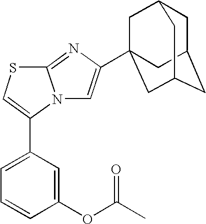

The title compound is synthesized according to General Procedure 2 by reacting trifluoromethanesulfonic acid 6-adamantan-1-yl-imidazo[2,1-b]thiazol-3-yl ester with acetic acid 3-(4,4,5,5-tetramethyl-[1,3,2]dioxaborolan-2-yl)-phenol ester.

Physical characteristics are as follows:

Mp 199-201° C.; ¹H NMR (DMSO-d₆, TMS) δ: 1.74, 1.96, 2.05, 2.32, 6.34, 7.57, 7.60-7.70, 7.81.

### EXAMPLE 58

### 5-(6-Adamantan-1-yl-imidazo[2,1-b]thiazole-3-yl)-2-methoxy-phenylamine hydrochloride

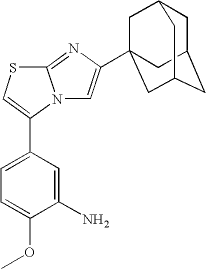

The title compound is synthesized according to General Procedure 2 by reacting trifluoromethanesulfonic acid 6-adamantan-1-yl-imidazo[2,1-b]thiazol-3-yl ester with 2-methoxy-5-(4,4,5,5-tetramethyl-[1,3,2]dioxaborolan-2-yl)-aniline.

Physical characteristics are as follows:

Mp 204° C. (decomp.); ¹H NMR (DMSO-d₆, TMS) δ: 1.72, 1.97, 2.05, 3.91, 7.20, 7.42, 7.50, 7.59, 7.87.

### EXAMPLE 59

### 3-(6-tert-Butyl-imidazo[2,1-b]thiazole-3-yl)benzoic acid methyl ester hydrochloride

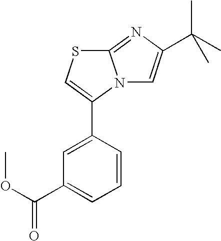

The title compound is synthesized according to General Procedure 2 by reacting trifluoromethanesulfonic acid 6-tert-butylimidazo[2,1-b]thiazol-3-yl ester with 3-methoxycarbonylbenzeneboronic acid.

Physical characteristics are as follows:

Mp 135° C. (decomp.); ¹H NMR (CDCl₃, TMS) (free base) δ: 1.35, 3.96, 6.77, 7.31, 7.59, 7.82, 8.11, 8.29.

### EXAMPLE 60

### Acetic acid, 3-(6-Adamantan-1-yl-imidazo[2,1-b]thiazol-3-yl)benzyl ester hydrochloride

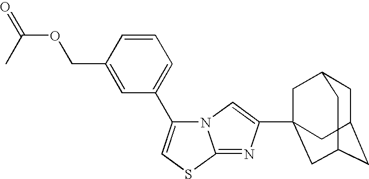

The title compound is synthesized by acetylation of [3-(6-adamantan-1-yl-imidazo[2,1-b]thiazole-3-yl)phenyl]-methanol (Example 53) with acetic anhydride in pyridine.

Physical characteristics are as follows:

Mp 135° C. (decomp.); ¹H NMR (CDCl₃, TMS) δ: 1.80, 2.07, 2.14, 5.20, 7.19, 7.57-7.63

### EXAMPLE 61

### 2-(6-Adamantan-1-yl-imidazo[2,1-b]thiazol-3-yl)phenylamine

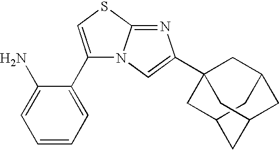

The title compound is synthesized according to General Procedure 2 by reacting trifluoromethanesulfonic acid 6-adamantan-1-yl-imidazo[2,1-b]thiazol-3-yl ester with 2-(4,4,5,5-tetramethyl-[1,3,2]dioxaborolan-2-yl)-phenylamine.

Physical characteristics are as follows:

Mp 160-162° C.; ¹H NMR (CDCl₃, TMS) δ: 1.76, 1.95, 2.04, 3.87, 6.72,6.79-6.88, 7.03, 7.23-7.31

### EXAMPLE 62

### N[3-(6-Adamantan-1-yl-imidazo[2,1-b]thiazol-3-yl)-phenyl]-methanesulfonamide

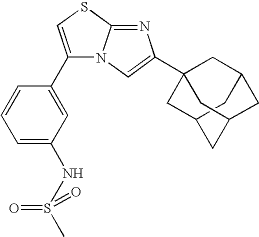

The title compound is synthesized according to General Procedure 2 by reacting trifluoromethanesulfonic acid 6-adamantan-1-yl-imidazo[2,1-b]thiazol-3-yl ester with N-[3-(4,4,5,5-tetramethyl-[1,3,2]dioxaborolan-2-yl)-phenyl]-methanesulfonamide

Physical characteristics are as follows:

Mp 165° C. (decomp.); ¹H NMR (CDCl₃, TMS) δ: 1.77, 1.98, 2.04, 3.09, 6.67,7.26-7.33, 7.46-7.50, 7.56

### EXAMPLE 63

### 3-(6-Adamantan-1-yl-imidazo[2,1-b]thiazol-3-yl)-benzonitrile hydrochloride

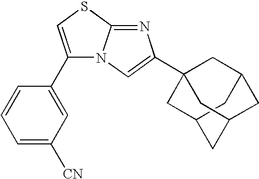

The title compound is synthesized according to General Procedure 2 by reacting trifluoromethanesulfonic acid 6-adamantan-1-yl-imidazo[2,1-b]thiazol-3-yl ester with 3-(4,4,5,5-tetramethyl-[1,3,2]dioxaborolan-2-yl)-benzonitrile.

Physical characteristics are as follows:

Mp 150-153° C.; ¹H NMR (CDCl₃, TMS), (free base) δ: 1.78, 1.98, 2.08, 6.82, 7.61-7.79, 7.87-7.92

### EXAMPLE 64

### Acetic acid, 3-acetoxy-4-(6-Adamantan-1-yl-imidazo[2,1-b]thiazol-3-yl)phenyl ester hydrochloride

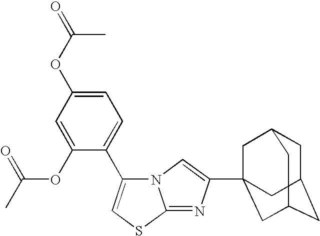

The title compound is synthesized by acetylation of 4-(6-adamantan-1-yl-imidazo[2,1-b]thiazole-3-yl)benzene-1,3-diol (Example 52) with acetic anhydride in pyridine.

Physical characteristics are as follows:

Mp 203-205° C.; ¹H NMR (DMSO-d₆, TMS), δ: 1.72, 1.93, 2.04, 2.11, 2.32, 7.26-7.34, 7.55-7.57, 7.78

### EXAMPLE 65

### Acetic acid, 2-acetoxy-4-(6-Adamantan-1-yl-imidazo[2,1-b]thiazol-3-yl)phenyl ester hydrochloride

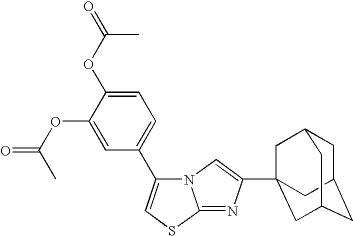

The title compound is synthesized by acetylation of 4-(6-adamantan-1-yl-imidazo[2,1-b]thiazol-3-yl)benzene-1,2-diol (Example 66) with acetic anhydride in pyridine.

Physical characteristics are as follows:

Mp 185-187° C.; ¹H NMR (CDCl₃, TMS) (free base), δ: 1.77, 1.97, 2.06, 2.32, 2.34, 6.71, 7.24-7.35, 7.48-7.54

### EXAMPLE 66

### 4-(6-Adamantan-1-yl-imidazo[2,1-b]thiazol-3-yl)benzene-1,2-diol hydrochloride

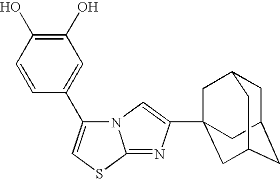

The title compound is synthesized by demethylation of 6-adamantan-1-yl-3-(3,4-dimethoxyphenyl)-imidazol[2,1-b]thiazol (Example 7) with boron tribromide in dichloromethane.

Physical characteristics are as follows:

Mp 303-305° C.; ¹H NMR (DMSO-d₆, TMS) (free base) δ: 1.71, 1.89, 2.00, 6.86, 7.00, 7.08, 7.09, 7.43

### EXAMPLE 67

### Acetic acid, 4-acetoxy-3-(6-Adamantan-1-yl-imidazo[2,1-b]thiazol-3-yl)phenyl ester hydrochloride

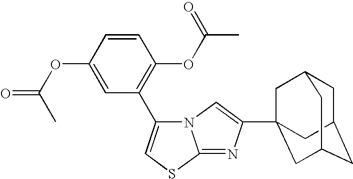

The title compound is synthesized by acetylation of 2-(6-adamantan-1-yl-imidazo[2,1-b]thiazol-3-yl)benzene-1,4-diol (example 68) with acetic anhydride in pyridine.

Physical characteristics are as follows:

Mp 153-155° C.; ¹H NMR (CDCl₃, TMS) (free base) δ: 1.76, 1.95, 2.08, 2.33, 6.74, 7.06, 7.25-7.35

### EXAMPLE 68

### 2-(6-Adamantan-1-yl-imidazo[2,1-b]thiazol-3-yl)benzene-1,4-diol hydrochloride

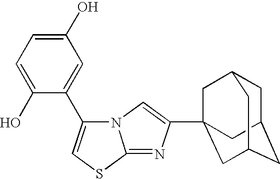

The title compound is synthesized by demethylation of 6-adamantan-1-yl-3-(2,5-dimethoxyphenyl)-imidazo[2,1-b]thiazole (Example 4) with boron tribromide in dichloromethane.

Physical characteristics are as follows:

Mp 290-293° C.; ¹H NMR (DMSO-d₆, TMS) δ: 1.72, 1.93, 2.05, 6.80-6.90, 7.50, 7.57

### EXAMPLE 69

### 6-Adamantan-1-yl-3-pyridin-2-yl-imidazo[2,1-b]thiazole dihydrochloride

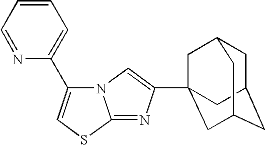

The title compound is synthesized according to General Procedure 2 by reacting trifluoromethanesulfonic acid 6-adamantan-1-yl-imidazo[2,1-b]thiazol-3-yl ester with 2-tributylstannanyl-pyridine

Physical characteristics are as follows:

Mp 218-220° C.; ¹H NMR (DMSO-d₆, TMS) δ: 1.72, 1.97, 2.04, 8.06, 8.18-8.29, 8.90.

### EXAMPLE 70

### 6-Adamantan-1-yl-3-(4-amino-3-methoxyphenyl)imidazo[2,1-b]thiazole hydrochloride

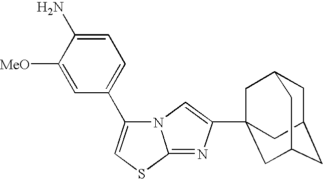

The title compound is synthesized according to General Procedure 2 by reacting trifluoromethanesulfonic acid 6-adamantan-1-yl-imidazo[2,1-b]thiazol-3-yl ester with 2-methoxy-4-(4,4,5,5-tetramethyl-[1,3,2]dioxaborolan-2-yl)-phenylamine

Physical characteristics are as follows:

Mp 160° C. (decomp); ¹H NMR (CDCl₃, TMS) (free base) δ: 1.77, 1.98, 2.04, 3.91, 4.04, 6.52, 6.78, 6.99, 7.07.

### EXAMPLE 71

### 6-Adamantan-1-yl-3-(3-chlorophenyl)-imidazo[2,1-b]thiazole hydrochloride

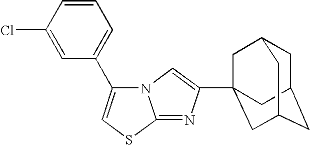

The title compound is synthesized according to General Procedure 2 by reacting trifluoromethanesulfonic acid 6-adamantan-1-yl-imidazo[2,1-b]thiazol-3-yl ester with 3-chlorobenzeneboronic acid

Physical characteristics are as follows:

Mp 179-181° C. (decomp); ¹H NMR (DMSO-d₆, TMS) δ: 1.72, 1.96, 2.04, 7.59-7.63, 7.34, 7.81, 7.82, 7.93.

### EXAMPLE 72

### 3-(6-Adamantan-1-yl-imidazo[2,1-b]thiazol-3-yl)benzoic acid methyl ester hydrochloride

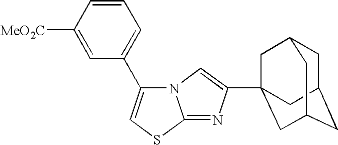

The title compound is synthesized according to General Procedure 2 by reacting trifluoromethanesulfonic acid 6-adamantan-1-yl-imidazo[2,1-b]thiazol-3-yl ester with 3-(4,4,5,5-tetramethyl-[1,3,2]dioxaborolan-2-yl)-benzoic acid methyl ester

Physical characteristics are as follows:

Mp 155° C. (decomp); ¹H NMR (CDCl₃, TMS) (free base) δ: 1.78, 1.99, 2.07, 3.97, 6.79, 7.29, 7.60, 7.83, 8.12, 8.31.

### EXAMPLE 73

### 6-Adamantan-1-yl-3-(3,5-dimethoxyphenyl)-imidazo[2,1-b]thiazole hydrochloride

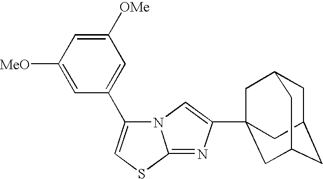

The title compound is synthesized according to General Procedure 1, Method B by reacting 2-amino-4-(3,5-dimethoxyphenyl)thiazole with 1-bromoacetyladamantane.

Physical characteristics are as follows:

Mp 165-167° C.; ¹H NMR (CDCl₃, TMS) (free base) δ: 1.79, 2.07, 2.11, 3.87, 6.63, 6.68, 7.13, 7.30.

### EXAMPLE 74

### N-[5-(6-Adamantan-1-yl-imidazo[2,1-b]thiazol-3-yl)-2-methoxy-phenyl]-acetamide hydrochloride

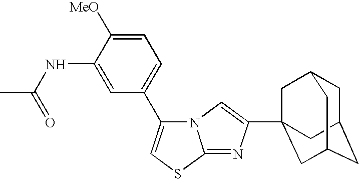

The title compound is synthesized by acetylation of 5-(6-adamantan-1-yl-imidazo[2,1-b]thiazol-3-yl)-2-methoxy-phenylamine (Example 58) with acetic anhydride in pyridine.

Physical characteristics are as follows:

Mp 153-155° C.; ¹H NMR (CDCl₃, TMS) (free base) δ: 1.76, 2.00, 2.03, 2.24, 3.95, 6.65, 6.97, 7.31, 7.37, 7.81, 8.71.

### EXAMPLE 75

### 6-Adamantan-1-yl-2-phenyl-imidazo[2,1-b]thiazole hydrochloride

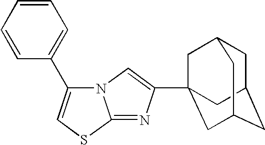

The title compound is synthesized according to General Procedure 1, Method B by reacting 2-amino-4-phenylthiazole with 1-bromoacetyladamantane.

Physical characteristics are as follows:

Mp 195-196° C.; ¹H NMR (DMSO-d₆, TMS) δ: 1.73, 1.94, 2.05, 7.50, 7.71, 7.81, 8.61.

Pure stereoisomeric forms of the compounds and the intermediates of this invention may be obtained by the application of art-known procedures. Diastereomers may be separated by physical separation methods such as selective crystallization and chromatographic techniques, e.g. liquid chromatography using chiral stationary phases. Enantiomers may be separated from each other by selective crystallization of their diastereomeric salts with optically active acids. Alternatively, enantiomers may be separated by chromatographic techniques using chiral stationary phases. Said pure stereoisomeric forms may also be derived from the corresponding pure stereoisomeric form of appropriate starting materials, provided that the reaction occurs stereoselectively. Stereoisomeric forms of Formula I are obviously intended to be included within the scope of this invention.

### Addition Salts

For therapeutic use, salts of the compounds of Formula I are those wherein the counterion is pharmaceutically acceptable. However, salts of acids and bases, which are non-pharmaceutically acceptable, may also find use, for example, in the preparation and purification of pharmaceutically acceptable compounds. All salts whether pharmaceutically acceptable or not are included within the ambit of the present invention. The pharmaceutically acceptable salts as mentioned above are meant to comprise the therapeutically active non-toxic salt forms, which the compounds of Formula I are able to form. The latter can conveniently be obtained by treating the base form with such appropriate acids as inorganic acids, e.g. hydrohalic acids such as hydrochloric, hydrobromic and the like; sulfuric acid; nitric acid; phosphoric acid and the like; or organic acids such as acetic, propanoic, hydroxyacetic, 2-hydroxypropanoic, oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfonic, 2-hydroxybenzoic, 4-amino-2-hydroxybenzioc and the like acids. Conversely, the salt form can be converted by treatment with alkali into the free base form.

### Pharmaceutical Compositions

The active ingredients of the Group I mGluR modulators of the present invention, together with one or more conventional adjuvants, carriers, or diluents, may be placed into the form of pharmaceutical compositions and unit dosages thereof, and in such form may be employed as solids, such as coated or uncoated tablets or filled capsules, liquids, such as solutions, suspensions, emulsions, elixirs, or capsules filled with the same, or thin films/flash doses, all for oral use; in the form of suppositories or capsules for rectal administration or in the form of sterile injectable solutions for parenteral (including intravenous or subcutaneous) use. Such pharmaceutical compositions and unit dosage forms thereof may comprise conventional or new ingredients in conventional or special proportions, with or without additional active compounds or principles, and such unit dosage forms may contain any suitable effective amount of the active ingredient of the Group I mGluR modulators of the present invention commensurate with the intended daily dosage range to be employed. Tablets containing one (1) to one hundred (100) milligrams of active ingredient or, more broadly, zero point five (0.5) to five hundred (500) milligrams per tablet, are accordingly suitable representative unit dosage forms.

The term “carrier” applied to pharmaceutical compositions of the invention refers to a diluent, excipient, or vehicle with which a Group I mGluR modulator of the present invention is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. A. R. Gennaro, 20^{th} Edition, describes suitable pharmaceutical carriers in “Remington: The Science and Practice of Pharmacy”.

### Method of Treating

Due to their high degree of activity and their low toxicity, together presenting a most favorable therapeutic index, the active principles of the invention may be administered to a subject, e.g., a living animal (including a human) body, in need thereof, for the treatment, alleviation, or amelioration, palliation, or elimination of an indication or condition which is susceptible thereto, or representatively of an indication or condition set forth elsewhere in this application, preferably concurrently, simultaneously, or together with one or more pharmaceutically-acceptable excipients, carriers, or diluents, especially and preferably in the form of a pharmaceutical composition thereof, whether by oral, rectal, or parental (including intravenous and subcutaneous) or in some cases even topical route, in an effective amount. Suitable dosage ranges are 1-1000 milligrams daily, preferably 10-500 milligrams daily, and especially 50-500 milligrams daily, depending as usual upon the exact mode of administration, form in which administered, the indication toward which the administration is directed, the subject involved and the body weight of the subject involved, and the preference and experience of the physician or veterinarian in charge.

The term “treat” is used herein to mean to relieve or alleviate at least one symptom of a disease in a subject. Within the meaning of the present invention, the term “treat” also denotes to arrest, delay the onset (i.e., the period prior to clinical manifestation of a disease) and/or reduce the risk of developing or worsening a disease.

The term “combination” is used herein to define a single pharmaceutical composition (formulation) comprising a Group I mGluR modulator of the present invention and an NMDA receptor antagonist, in a formulation known in the art, or two separate pharmaceutical compositions (formulations), one comprising a Group I mGluR modulator of the present invention as formulated above and one comprising an NMDA receptor antagonist in a formulation known in the art, to be administered conjointly.

Within the meaning of the present invention, the term “conjoint administration” is used to refer to administration of a Group I mGluR modulator of the present invention and an NMDA receptor antagonist simultaneously in one composition, or simultaneously in different compositions, or sequentially. For the sequential administration to be considered “conjoint”, however, the Group I mGluR modulator of the present invention and the NMDA receptor antagonist must be administered separated by a time interval that still permits the resultant beneficial effect in a mammal. For example, the Group I mGluR modulator of the present invention and the NMDA receptor antagonist must be administered on the same day (e.g., each—once or twice daily), preferably within an hour of each other, and most preferably simultaneously.

The term “therapeutically effective” applied to dose or amount refers to that quantity of a compound or pharmaceutical composition that is sufficient to result in a desired activity upon administration to a living animal body in need thereof.

The Group I mGluR modulators of the present invention may be administered orally, topically, parenterally, or mucosally (e.g., buccally, by inhalation, or rectally) in dosage unit formulations containing conventional non-toxic pharmaceutically acceptable carriers. It is usually desirable to use the oral route. The Group I mGluR modulators of the present invention may be administered orally in the form of a capsule, a tablet, or the like (see Remington: The Science and Practice of Pharmacy, 20^{th} Edition). The orally administered medicaments may be administered in the form of a time-controlled release vehicle, including diffusion-controlled systems, osmotic devices, dissolution-controlled matrices, and erodible/degradable matrices.

For oral administration in the form of a tablet or capsule, the Group I mGluR modulator active component may be combined with a non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like. For oral administration in liquid form, the Group I mGluR modulator active components may be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) may also be added to stabilize the dosage forms.

The tablets may be coated by methods well known in the art. The Group I mGluR modulators of the present invention may be also introduced in beads, microspheres or microcapsules, e.g., fabricated from polyglycolic acid/lactic acid (PGLA). Liquid preparations for oral administration may take the form of, for example, solutions, syrups, emulsions or suspensions, or they may be presented as a dry product for reconstitution with water or other suitable vehicle before use. Preparations for oral administration may be suitably formulated to give controlled or postponed release of the active compound.

The Group I mGluR modulators of the present invention may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines, as is well known.

The Group I mGluR modulators of the present invention may also be delivered by the use of monoclonal antibodies as individual carriers to which the compound molecules are coupled. The Group I mGluR modulators may also be coupled with soluble polymers as targetable drug carriers. Such polymers include polyvinyl-pyrrolidone, pyran copolymer, polyhydroxy-propyl methacrylamide-phenol, polyhydroxy-ethyl-aspartamide-phenol, or polyethyleneoxide-polylysine substituted with palmitoyl residues. Furthermore, the Group I mGluR modulators may be coupled to a class of biodegradable polymers useful in achieving controlled release of a drug, for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

For administration by inhalation, the Group I mGluR modulators of the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges of, e.g., gelatin for use in an inhaler or insufflator can be formulated containing a powder mix of the compound and a suitable powder base such as lactose or starch.

The formulations comprising the Group I mGluR modulators of the present invention may be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection can be presented in unit dosage form, e.g., in ampoules or in multi-dose containers, with an added preservative. The compositions can take such forms as excipients, suspensions, solutions, or emulsions in oily or aqueous vehicles, and can contain formulatory agents such as suspending, stabilizing and/or dispersing agents. Alternatively, the active ingredient of the Group I mGluR modulators of the present invention can be in powder form for reconstitution with a suitable vehicle, e.g., sterile pyrogen-free water, before use.

The Group I mGluR modulators of the present invention may also be formulated for rectal administration, e.g., as suppositories or retention enemas (e.g., containing conventional suppository bases such as cocoa butter or other glycerides).

The compositions comprising Group I mGluR modulators of the present invention may, if desired, be presented in a pack or dispenser device, which may contain one or more unit dosage forms containing the active ingredient and/or may contain different dosage levels to facilitate dosage titration. The pack may, for example, comprise metal or plastic foil, such as a blister pack. The pack or dispenser device may be accompanied by instructions for administration. The Group I mGluR modulators of the present invention formulated in a compatible pharmaceutical carrier may also be prepared, placed in an appropriate container, and labeled for treatment of an indicated condition.

As disclosed herein, the dose of the components in the compositions of the present invention is determined to ensure that the dose administered continuously or intermittently will not exceed an amount determined after consideration of the results in test animals and the individual conditions of a patient. A specific dose naturally varies depending on the dosage procedure, the conditions of a patient or a subject animal such as age, body weight, sex, sensitivity, feed, dosage period, drugs used in combination, seriousness of the disease. The appropriate dose and dosage times under certain conditions can be determined by the test based on the above-described indices but may be refined and ultimately decided according to the judgment of the practitioner and each patient's circumstances (age, general condition, severity of symptoms, sex, etc.) according to standard clinical techniques.

Toxicity and therapeutic efficacy of the compositions of the invention can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it may be expressed as the ratio LD₅₀/ED₅₀. Compositions that exhibit large therapeutic indices are preferred.

### Examples of Representative Pharmaceutical Compositions

With the aid of commonly used solvents, auxiliary agents and carriers, the reaction products can be processed into tablets, coated tablets, capsules, drip solutions, suppositories, injection and infusion preparations, and the like and can be therapeutically applied by the oral, rectal, parenteral, and additional routes. Representative pharmaceutical compositions follow.

(a) Tablets suitable for oral administration, which contain the active ingredient, may be prepared by conventional tabletting techniques.

(b) For suppositories, any usual suppository base may be employed for incorporation thereinto by usual procedure of the active ingredient, such as a polyethyleneglycol which is a solid at normal room temperature but which melts at or about body temperature.

(c) For parental (including intravenous and subcutaneous) sterile solutions, the active ingredient together with conventional ingredients in usual amounts are employed, such as for example sodium chloride and double-distilled water q.s., according to conventional procedure, such as filtration, aseptic filling into ampoules or IV-drip bottles, and autoclaving for sterility.

Other suitable pharmaceutical compositions will be immediately apparent to one skilled in the art.

### FORMULATION EXAMPLES

The following examples are again given by way of illustration only and are not to be construed as limiting.

### Example 1

### Tablet Formulation

A suitable formulation for a tablet containing 10 milligrams of active ingredient is as follows:

| | mg |
|---|---|
| Active Ingredient | 10 |
| Lactose | 61 |
| Microcrystalline Cellulose | 25 |
| Talcum | 2 |
| Magnesium stearate | 1 |
| Colloidal silicon dioxide | 1 |

### Example 2

### Table Formulation

Another suitable formulation containing 100 mg is as follows:

| | mg |
|---|---|
| Active Ingredient | 100 |
| Polyvinylpyrrolidone, crosslinked | 10 |
| Potato starch | 20 |
| Polyvinylpyrrolidone | 19 |
| Magnesium stearate | 1 |
| Microcrystalline Cellulose | 50 |
| Film coated and colored. | |

The film coating material consists of:

| | |
|---|---|
| Hypromellose | 10 |
| Microcryst. Cellulose | 5 |
| Talcum | 5 |
| Polyethylene glycol | 2 |
| Color pigments | 5 |

### Example 3

### Capsule Formulation

A suitable formulation for a capsule containing 50 milligrams of active ingredient is as follows:

| | mg |
|---|---|
| Active Ingredient | 50 |
| Corn starch | 26 |
| Dibasic calcium phosphate | 50 |
| Talcum | 2 |
| Colloidal silicon dioxide | 2 |

filled in a gelatin capsule.

### Example 4

### Sulution for Injection

A suitable formulation is as follows:

| | | |
|---|---|---|
| Active Ingredient | mg | 10 |
| Sodium chloride | mg | q.s. |
| Water for Injection | mL | add 1.0 |

### Example 5

### Liquid Oral Formulation

A suitable formulation for 1 liter of an oral solution containing 2 milligrams of active ingredient in one milliliter of the mixture is as follows:

| | mg |
|---|---|
| Active Ingredient | 2 |
| Saccharose | 250 |
| Glucose | 300 |
| Sorbitol | 150 |
| Orange flavor | 10 |
| Colorant | q.s. |
| Purified water | add 1000 mL |

### Example 6

### Liquid Oral Formulation

Another suitable formulation for 1 liter of a liquid mixture containing 20 milligrams of active ingredient in one milliliter of the mixture is as follows:

| | G |
|---|---|
| Active Ingredient | 20.00 |
| Tragacanth | 7.00 |
| Glycerol | 50.00 |
| Saccharose | 400.00 |
| Methylparaben | 0.50 |
| Propylparaben | 0.05 |
| Black currant-flavor | 10.00 |
| Soluble Red color | 0.02 |
| Purified water | add 1000 mL |

### Example 7

### Liquid Oral Formulation

Another suitable formulation for 1 of a liquid mixture containing 2 milligrams of active ingredient in one milliliter of the mixture is as follows:

| | G |
|---|---|
| Active Ingredient | 2 |
| Saccharose | 400 |
| Bitter orange peel tincture | 20 |
| Sweet orange peel tincture | 15 |
| Purified water | add 1000 mL |

### Example 8

### Aerosol Formulation

180 g aerosol solution contain:

| | G |
|---|---|
| Active Ingredient | 10 |
| Oleic acid | 5 |
| Ethanol | 81 |
| Purified Water | 9 |
| Tetrafluoroethane | 75 |

15 ml of the solution are filled into aluminum aerosol cans, capped with a dosing valve, purged with 3.0 bar.

### Example 9

### TDS Formulation

100 g solution contain:

| | G |
|---|---|
| Active Ingredient | 10.0 |
| Ethanol | 57.5 |
| Propyleneglycol | 7.5 |
| Dimethylsulfoxide | 5.0 |
| Hydroxyethylcellulose | 0.4 |
| Purified water | 19.6 |

1.8 ml of the solution are placed on a fleece covered by an adhesive backing foil. The system is closed by a protective liner which will be removed before use.

### Example 10

### Nanoparticle Formulation

10 g of polybutylcyanoacrylate nanoparticles contain:

| | G |
|---|---|
| Active Ingredient | 1.00 |
| Poloxamer | 0.10 |
| Butylcyanoacrylate | 8.75 |
| Mannitol | 0.10 |
| Sodium chloride | 0.05 |

Polybutylcyanoacrylate nanoparticles are prepared by emulsion polymerization in a water/0.1 N HCl/ethanol mixture as polymerizsation medium. The nanoparticles in the suspension are finally lyophilized under vacuum.

### Pharmacology

The active principles of the present invention, and pharmaceutical compositions thereof and method of treating therewith, are characterized by unique and advantageous properties, rendering the “subject matter as a whole”, as claimed herein, unobvious. The compounds and pharmaceutical compositions thereof exhibit, in standard accepted reliable test procedures, the following valuable properties and characteristics:

### Methods

### Binding Assays for the Characterization of mGluR5 Antagonist Properties

### [³H]MPEP (2-methyl-6-(phenylethynyl)pyridine) Binding to Transmembrane Allosteric Modulatory Sites of mGluR5 Receptors in Cortical Membranes

### Preparation of Rat Cortical Membranes:

Male Sprague-Dawley rats (200-250 g) are decapitated and their brains are removed rapidly. The cortex is dissected and homogenized in 20 volumes of ice-cold 0.32 M sucrose using a glass-Teflon homogenizer. The homogenate is centrifuged at 1000×g for 10 min. The pellet is discarded and the supernatant centrifuged at 20,000×g for 20 min. The resulting pellet is re-suspended in 20 volumes of distilled water and centrifuged for 20 min at 8000×g. Then the supernatant and the buffy coat are centrifuged at 48,000×g for 20 min in the presence of 50 mM Tris-HC, pH 8.0. The pellet is then re-suspended and centrifuged two to three more times at 48,000×g for 20 min in the presence of 50 mM Tris-HCl, pH 8.0. All centrifugation steps are carried out at 4° C. After resuspension in 5 volumes of 50 mM Tris-HCl, pH 8.0 the membrane suspension is frozen rapidly at −80° C.

On the day of assay the membranes are thawed and washed four times by resuspension in 50 mM Tris-HCI, pH 8.0 and centrifugation at 48,000×g for 20 min. and finally re-suspended in 50 mM Tris-HCl, pH 7.4. The amount of protein in the final membrane preparation (250-500 μg/ml) is determined according to the method of Lowry (Lowry O. H. et al., 1951, J. Biol. Chem., 193, 256-275).

### [³H]MPEP Assay

Incubations are started by adding (³H)-MPEP (50.2 Ci/mmol, 5 nM, Tocris) to vials with 125-250 μg protein (total volume 0.5 ml) and various concentrations of the agents. The incubations are continued at room temperature for 60 min (equilibrium is achieved under the conditions used). Non-specific binding is defined by the addition of unlabeled MPEP (10 μM). Incubations are terminated using a Millipore filter system. The samples are rinsed twice with 4 ml of ice-cold assay buffer over glass fibre filters (Schleicher & Schuell) under a constant vacuum. Following separation and rinse, the filters are placed into scintillation liquid (5 ml Ultima Gold) and radioactivity retained on the filters is determined with a conventional liquid scintillation counter (Hewlett Packard, Liquid Scintillation Analyser).

### Characterization

Specific binding is extremely high i.e. normally >85% and essentially independent of buffer (Tris or HEPES oth 50 mM) and pH (6.8-8.9). There is a clear saturable protein dependence and the chosen protein concentration used for subsequent assays (250-500 μg/ml) is within the linear portion of this dependence. Cold MPEP displaces hot ligand with an IC₅₀ of 18.8±4.1 nM. The Kd of (³H)-MPEP of 13.6 nM is determined by Scatchard analysis and used according to the Cheng Prussoff relationship to calculate the affinity of displacers as Kd values (IC₅₀ of cold MPEP equates to a Ki of 13.7 nM). Bₘₐₓ is 0.56 pm/mg protein.

### Functional Assay of MGLUR5 Receptors

### Materials and Methods

### Astrocyte Culture

Primary astrocyte cultures are prepared from cortices of newborn rats as described by Booher and Sensenbrenner (1972, Neurobiology 2(3):97-105). Briefly, Sprague-Dawley rat pups (2-4 d old) are decapitated and neocortices are dissected, disintegrated with a nylon filter (poresize 80 μm) and carefully triturated. The cell suspension is plated on poly-D-lysine precoated flasks (Costar) and cultivated in Dulbecco's Modified Eagle's Medium (DMEM, InVitrogen) supplemented with 10% heat inactivated fetal calf serum (FCSᵢ, Sigma), 4 mM glutamine (Biochrom) and 50 μg/mL gentamycin (Biochrom) at 37° C. in a humidified atmosphere of 5% CO₂/95% air for 7 d with exchanging the medium at day 2.

After 7 DIV, cells are shaken overnight at 250 rpm to remove oligodendrocytes and microglia. The next day, astrocytes are rinsed twice with CMF-PBS, trypsinized and subplated on poly-D-lysine precoated 96-well plates (Becton Dickinson #6516 or #6640) at a density of 40,000-45,000 cells/well. 24 h after establishing the secondary culture the astrocytes are rinsed with PBS⁺⁺ and fed with astrocyte-defined medium (ADM) consisting of DMEM containing 1× G5-supplement (InVitrogen), 0.5 μg/mL heparan sulfate (Sigma), and 1.5 μg/mL fibronectin (Sigma) (Miller et al., (1993) Brain Res. 618(1):175-8). 3 d later the medium is exchanged and the cells incubated for another 2-3 d, so that at the time of experiments astrocytes are 14-15 DIV.

### Immunocytochemistry

Immunostaining is performed to confirm the presence of classical astrocytic markers such as GFAP as well the expression of mGluR5 receptors.

### Accumulation of [³H]-Inositol Phosphates

After astrocytes are cultured for 12 d ADM is removed and inositol-free DMEM (MP Biomedicals) supplemented with [³H]myo-inositol (0.5 μCi/well; Perkin Elmer), and the ADM chemicals is added. After 48 h the medium is replaced with 100 μL Locke's buffer (plus 20 mM Li⁺, pH 7.4) and incubated for 15 min at 37° C. before replacement with agonists/antagonists in Locke's buffer. The incubation (45 min at 37° C.) is terminated by replacing the Locke's solutions with 100 μL 0.1 M HCl (10 min on ice). The 96 well plates can be frozen at −20° C. at this stage until further analysis. Home made resin exchange columns (AG1-X8 Biorad, 140-14444) are used to separate labeled inositol phosphates by elution with 1 mL of 1 M ammonium formate/0.1 M formic acid into 24-well visiplates (Perkin Elmer). Scintillation liquid (UltimaFlow AF, Perkin Elmer) is added, the plate sealed and vortexed before radioactivity is determined by conventional liquid scintillation counting (Microbeta,Perkin Elmer) as disintegration per minute (DPM).

Alternatively, on the day of assay, columns are washed with 1 mL of 0.1M formic acid followed by 1 mL of distilled water. The contents of each assay well are then added to one column and washed with 1 mL distilled water followed by 1 mL of 5 mM sodium tetraborate/60 mM sodium formate. The retained radioactive inositol phosphates are then eluted with 2×1 mL of 1M ammonium formate/0.1M formic acid into 24-well visiplates. Scintillation liquid (UltimaFlow AF, Perkin Elmer) is added, the plate sealed and vortexed before radioactivity is determined by conventional liquid scintillation counting (Microbeta, Perkin Elmer) as disintegration per minute (DPM).

### Calcium FLIPR Studies

Cultured astrocytes express mGluR5 receptors as shown by immunostaining. The increase of intracellular calcium after stimulation with the mGluR5 agonist DHPG or L-quisqualate is measured using the fluorometric imaging plate reader (FLIPR) and the Ca-Kit (both Molecular Devices, CA). Prior to addition of agonist or antagonist the medium is aspirated and cells are loaded for 2 h at RT with 150 μL of loading buffer consisting of Ca-sensitive dye (MD # R8033) reconstituted in sodium chloride (123 mM), potassium chloride (5.4 mM), magnesium chloride (0.8 mM), calcium chloride (1.8 mM), D-glucose (15 mM), and HEPES (20 mM), pH 7.3. Subsequently, plates are transferred to FLIPR to detect calcium increase with the addition of DHPG (300 μM) or L-quisqualate (100 nM) measured as relative fluorescence units (RFU). If antagonists are tested, these compounds are pre-incubated for 10 min at RT before addition of the respective agonist.

For positive modulators, concentration-response curves for quisqualate are performed in the presence and absence of 10 μM modulator to determine the extent of potentiation/agonist potency increase. Thereafter, concentration-response curves for the positive modulator are performed in the presence of a fixed concentration of quisqualate showing the biggest window for potentiation (normally 10-30 nM).

### Data Analysis

The fluorescence signal increase after addition of agonist reflects the increase of intracellular calcium. Inconsistencies in the amount of cells per well are normalised by using the spatial uniformity correction of the FLIPR software. The mean of replicated temporal data (n=5) is calculated and used for graphical representation. For the evaluation of the pharmacology, the calcium changes in response to different concentrations of agonist or antagonist are determined using a maximum minus minimum (MaxMin) calculation.

All responses (DPM- or RFU-values) are determined as percentage of control (=maximum response at 100 nM quisqualate).

EC₅₀ and IC₅₀ are calculated according the logistic equation using GraFit 5.0 (Erithacus Software).

### Chemicals

Unless otherwise stated all chemicals are purchased from Sigma.

### Functional Assay of mGluR1 Receptors in Cerebellar Granule Cells—Radioactive Assay for Changes in IP3 LEVELS

### Preparation of Cerebellar Granule Cells

Cerebellar cortici are obtained from P8 postnatal Sprague Dawley rats, mechanically disrupted into small pieces with forceps and then transferred to Ca²⁺ and Mg²⁺ free Hank's buffered salt solution (HBSS-CMF) on ice. After three washes in HBSS-CMF, the tissue pieces are incubated 37° C. for 8 minutes in the presence of 0.25% trypsin/0.05% DNase. The enzymatic reaction is stopped with 0.016% DNAase/0.1% ovomucoid before centrifugation at 800 rpm for 5 minutes. The supernatant is replaced twice with NaHCO₃/HEPES-buffered basal Eagle medium (BME) plus 20 mM KCl. Cells are mechanically dissociated in 2 ml of BME by trituration through three Pasteur pipettes of successively decreasing tip diameter and then filtered through a 48 μM gauge filter. Cells are plated at a density of 150,000 cells in 50 μl in each well of poly-L-Lysin pre-coated 96 well plates (Falcon). The cells are nourished with BEM supplemented with 10% foetal calf serum, 2 mM glutamine (Biochrom), 20 mM KCl and gentamycin (Biochrom) and incubated at 36° C. with 5% CO₂ at 95% humidity. After 24 h, cytosine-β-D-arabinofuranoside (AraC, 10 μM) is added to the medium.

### IP₃ Assay With [³H]myo-inositol

After 6 DIV the culture medium is replaced completely with inositol free DMEM (ICN) containing [³H]myo-inositol (Perkin Elmer) at a final concentration of 0.5 μCi/100 μl/well and incubated for a further 48 hours. The culture medium in each well is replaced with 100 μL Locke's buffer (contains in (mM) NaCl (156), KCl (5.6), NaHCO₃ (3.6), MgCl₂ (1.0), CaCl₂ (1.3), Glucose (5.6), HEPES (10)) with additional (20 mM Li, pH 7.4) and incubated for 15 min at 37° C. Locke's buffer is replaced with agonists/agonists/putative mGluR1 ligands in Locke's buffer and incubated for 45 min. These solutions are then replaced by 100 μL 0.1M HCl in each well and incubated for a further 10 mins on ice. The 96 well plates can be frozen at −20° C. at this stage until further analysis.

Homemade resin exchange columns used to separate labeled inositol phosphates. For example, they are are prepared as follows. Empty Bio-Spin Chromatography columns (Biorad) are plugged with filter paper before filling with 1.1-1.2 ml of resin (AG1-X8 Biorad, 140-14444) suspended in 0.1 formic acid (24 g resin per 50 ml acid). The formic acid is allowed to run out before sealing the syringe tips and filling with 200-300 μL of 0.1 M formic acid before storage at 4° C. On the day of assay, columns are washed with 1 ml of 0.1 M formic acid followed by 1 ml of distilled water. The contents of each assay well are then added to one column and washed with 1 ml distilled water followed by 1 ml of 5 mM sodium tetraborate/60 mM sodium formate. The retained radioactive inositol phosphates are then eluted with 2*1 ml of 1 M ammonium formate/0.1 M formic acid into 24-well visiplates. Scintillation liquid (1.2 ml UltimaFlow AF) is added to each well and the plate sealed and vortexed before radioactivity is determined by conventional liquid scintillation counting (Microbeta,Perkin Elmer). Unless otherwise stated, all reagents are obtained from Sigma.

Compounds of the present invention have a potency (EC₅₀ or B-IC₅₀, respectively) range of about 0.5 nM to about 100 μM

### Conclusions

In conclusion, from the foregoing, it is apparent that the present invention provides novel, valuable, and unpredictable applications and uses of the compounds of the present invention, which compounds comprise the active principle according to the present invention, as well as novel pharmaceutical compositions thereof and methods of preparation thereof and of treating therewith, all possessed of the foregoing more specifically-enumerated characteristics and advantages.

The high order of activity of the active agent of the present invention and compositions thereof, as evidenced by the tests reported, is indicative of utility based on its valuable activity in human beings as well as in lower animals. Clinical evaluation in human beings has not been completed, however. It will be clearly understood that the distribution and marketing of any compound or composition falling within the scope of the present invention for use in human beings will of course have to be predicated upon prior approval by governmental agencies, such as the U.S. Federal Food and Drug Administration, which are responsible for and authorized to pass judgment on such questions.

The instant imidazothiazole derivatives represent a novel class of Group I mGluR modulators. In view of their potency, they will be useful therapeutics in a wide range of disorders, including CNS disorders, which involve excessive glutamate induced excitation.

These compounds accordingly find application in the treatment of the following disorders of a living animal body, especially a human: AIDS-related dementia, Alzheimer's disease, Creutzfeld-Jakob's syndrome, bovine spongiform encephalopathy (BSE) or other prion related infections, diseases involving mitochondrial dysfunction, diseases involving β-amyloid and/or tauopathy such as Down's syndrome, hepatic encephalopathy, Huntington's disease, motor neuron diseases such as amyotrophic lateral sclerosis (ALS), multiple sclerosis (MS), olivoponto-cerebellar atrophy, post-operative cognitive deficit (POCD), lupus disease, neuronal ceroid lipofuscinosis, neurodegenerative cerebellar ataxias, Parkinson's disease, Parkinson's dementia, mild cognitive impairment, dementia pugilisitca, vascular and frontal lobe dementia, cognitive impairment, eye injuries, eye diseases, eye disorders, glaucoma, retinopathy, macular degeneration, head and brain and spinal cord injuries/trauma, hypoglycaemia, hypoxia (e.g. perinatal), ischaemia (e.g. resulting from cardiac arrest, stroke, bypass operations or transplants), convulsions, epilepsy, myoclonic epilepsy, epileptic convulsions, temporal lobe epilepsy, glioma and other tumours, cancer, oral cancer, squamous cell carcinoma (SCC), oral squamous cell carcinoma (SSC), neoplasia, hyperplasia, dysplasia, cancer, carcinoma, sarcoma, oral cancer, squamous cell carcinoma (SCC), oral squamous cell carcinoma (SCC), lung cancer, lung adenocarcinoma, breast cancer, prostate cancer, gastric cancer, liver cancer, colon cancer, colorectal carcinoma, brain tumor, tumor of a nerve tissue, malignant glioma, astroglioma, neuroglioma, neuroblastoma, glioblastoma, medulloblastoma, cancer of skin cells, melanoma, malignant melanoma, epithelial neoplasm, lymphoma, myeloma, Hodgkin's disease, Burkett's lymphoma, leukemia, thymoma, inner ear insult (e.g. in tinnitus, sound or drug-induced), tinnitus, sound or drug-induced tinnitus, L-dopa-induced and tardive dyskinesias, L-dopa-induced dyskinesia in Parkinson's disease therapy, chorea, athetosis, stereotypy, ballism, Tic disorder, torticollis spasmodicus, blepharospasm, focal and generalized dystonia, nystagmus, hereditary cerebellar taxias, corticobasale degeneration, tremor, and essential tremor.

These compounds also find application in the treatment of the following disorders of a living animal body, especially a human: abuse and addiction (e.g., nicotine, alcohol, opiate, cocaine, amphetamine), obesity, amyotrophic lateral sclerosis (ALS), anxiety and panic disorders, attention deficit hyperactivity disorder (ADHD), attention deficit syndrome (ADS), restless leg syndrome, hyperactivity in children, autism, dementia (e.g. in Alzheimer's disease, Korsakoff syndrome, vascular dementia, dementia in HIV infections), major depressive disorder or depression (including that resulting from Borna virus infection) and bipolar manic-depressive disorder, drug tolerance (e.g. to opioids), movement disorders, dystonia, dyskinesia (e.g. L-Dopa-induced, tardive dyskinesia or dyskinesia in Huntington's disease), fragile-X syndrome, Huntington's chorea, irritable bowel syndrome (IBS), migraine, multiple sclerosis, muscle spasms, pain, chronic pain, acute pain, inflammatory pain, neuropathic pain, diabetic neuropathic pain (DNP), cancer pain, pain related to rheumatic arthritis, allodynia, hyperalgesia, nociceptive pain, post traumatic stress disorder, schizophrenia (positive, cognitive and negative symptoms), spasticity, Tourette's syndrome, urinary incontinence, vomiting, pruritic conditions (e.g. pruritis), sleep disorders, micturition disorders, neuromuscular disorder in the lower urinary tract, gastroesophageal reflux disease (GERD), lower esophageal sphincter (LES) disease, functional gastrointestinal disorders, dyspepsia, regurgitation, respiratory tract infection, bulimia nervosa, chronic laryngitis, asthma (e.g. reflux-related asthma), lung disease, eating disorders, obesity and obesity-related disorders, binge eating disorder, agoraphobia, generalized anxiety disorder, obsessive-compulsive disorder, panic disorder, social phobia, substance-induced anxiety disorder, delusional disorder, schizoaffective disorder, schizophreniform disorder, substance-induced psychotic disorder, delirium, or for cognitive enhancement and/or neuroprotection.

These compounds also find application in the treatment of indications in of a living animal body, especially a human, wherein a particular condition does not necessarily exist but wherein a particular physiological parameter may be improved through administration of the instant compounds, including cognitive enhancement.

The method-of-treating a living animal body with a compound of the invention, for the inhibition of progression or alleviation of the selected ailment therein, is as previously stated by any normally-accepted pharmaceutical route, employing the selected dosage which is effective in the alleviation of the particular ailment desired to be alleviated.

Use of the compounds of the present invention in the manufacture of a medicament for the treatment of a living animal for inhibition of progression or alleviation of selected ailments or conditions, particularly ailments or conditions susceptible to treatment with a Group I mGluR modulator is carried out in the usual manner comprising the step of admixing an effective amount of a compound of the invention with a pharmaceutically-acceptable diluent, excipient, or carrier, and the method-of-treating, pharmaceutical compositions, and use of a compound of the present invention in the manufacture of a medicament.

Representative pharmaceutical compositions prepared by admixing the active ingredient with a suitable pharmaceutically-acceptable excipient, diluent, or carrier, include tablets, capsules, solutions for injection, liquid oral formulations, aerosol formulations, TDS formulations, and nanoparticle formulations, thus to produce medicaments for oral, injectable, or dermal use, also in accord with the foregoing.

The present invention is not to be limited in scope by the specific embodiments described herein. Indeed, various modifications of the invention in addition to those described herein will become apparent to those skilled in the art from the foregoing description.

All patents, applications, publications, test methods, literature, and other materials cited herein are hereby incoporated by reference.

We claim:

1. A compound selected from those of Formula I 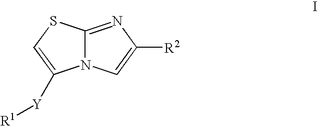
wherein
Y represents a single bond, CR³R⁴, C(═O), NR⁵, NHC(═O), C(═O)NH, OC(═O), C(═O)O, O, S, SO, or SO₂;
R¹ represents aryl, heteroaryl, arylC₁₋₆alkyl, arylC₂₋₆alkenyl, heteroarylC₁₋₆alkyl, heteroarylC₂₋₆alkenyl, C₁₋₆alkyl, or cycloC₃₋₁₂alkyl;
R² represents C₁₋₆alkyl, cycloC₃₋₁₂alkyl, Z-R^{6a}, C(═O)—R^{6b} or C(R⁷)(R⁸)—NR¹⁰R¹¹;
R³ and R⁴, which may be the same or different, each independently represent hydrogen, C₁₋₆alkyl, OH, C₁₋₆alkoxy, or halogen;
R⁵ represents hydrogen or C₁₋₆alkyl;
Z represents CR⁷R⁸, NR⁹, O, S, SO, or SO₂;
R^{6a} represents hydrogen, C₁₋₆alkyl, cycloC₃₋₁₂alkyl, aryl, heteroaryl, or heterocyclyl;
R^{6b} represents hydrogen, C₁₋₆alkyl, cycloC₃₋₁₂alkyl, or aryl;
R⁷ and R⁸, which may be the same or different, each independently represent hydrogen, C₁₋₆alkyl, C₁₋₆alkoxy, or halogen;
R⁹ represents hydrogen, C₁₋₆alkyl, cycloC₃₋₁₂alkyl, aryl, heteroaryl, heterocyclyl, or arylC₁₋₆alkyl
or
R^{6a} and R⁹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
R¹⁰ represents hydrogen, C₁₋₆alkyl, cycloC₃₋₁₂alkyl, aryl, heteroaryl, or heterocyclyl;
R¹¹ represents hydrogen, C₁₋₆alkyl, cycloC₃₋₁₂alkyl, aryl, heteroaryl, heterocyclyl, or arylC₁₋₆alkyl
or
R¹⁰ and R¹¹ together with the nitrogen atom to which they are attached may form a saturated mono-, bi-, spiro- or tricyclic ring system having from 3 to 12 carbon atoms, one or two of which may optionally be replaced by O, S, NH, or N—C₁₋₆alkyl, wherein the ring system is optionally substituted by one or more substituents, which may be the same or different, selected independently from C₁₋₆alkyl, C₁₋₆alkoxy, and halogen;
and optical isomers, polymorphs and pharmaceutically-acceptable acid and base addition salts, hydrates, and solvates thereof.

2. The compound of claim 1, wherein:
Y represents a single bond;
R¹ represents aryl or heteroaryl; and
R² represents cycloC₃₋₁₂alkyl.

3. The compound of claim 2, wherein R² represents adamantyl.

4. The compound of claim 1, wherein R² represents branched C₁₋₆alkyl.

5. The compound of claim 4, wherein R² represents 2-propyl, 2-butyl, iso-butyl, tert-butyl, 2-pentyl, 3-pentyl, iso-pentyl, 2-methylbutyl, tert-amyl, 2-hexyl, 3-hexyl, 1-methylpentyl, 2-methylpentyl, 3-methylpentyl, 4-methylpentyl, 2-dimethylbutyl, 3-dimethylbutyl, 2-ethylbutyl or 3-ethylbutyl.

6. The compound of claim 4, wherein R² represents tert-butyl.

7. The compound of claim 4, wherein:
Y represents a single bond; and
R¹ represents aryl, optionally substituted by one or more substituents, which may be the same or different, selected independently from C₁₋₆alkyl, C₁₋₆alkoxy, halogen, and C₁₋₆alkoxycarbonyl.

8. The compound of claim 7, wherein R¹ represents phenyl optionally substituted by one or more substituents selected from C₁₋₆alkyl, C₁₋₆alkoxy, halogen, and C₁₋₆alkoxycarbonyl.

9. The compound of claim 1, wherein:
Y represents a single bond;
R¹ represents aryl; and
R² represents Z-R^{6a}, wherein Z represents CR⁷R⁸ and R^{6a} represents aryl or cycloC₃₋₁₂alkyl.

10. The compound of claim 9, wherein R¹ represents phenyl optionally substituted by one or more substituents selected from C₁₋₆alkyl, C₁₋₆alkoxy, halogen, and C₁₋₆alkoxycarbonyl.

11. The compound of claim 9, wherein R⁷ and R⁸, which may be the same or different, each independently represent C₁₋₆alkyl and R^{6a} represents phenyl, optionally substituted by one or more substituents selected from C₁₋₆alkyl and halogen, or cycloC₃₋₁₂alkyl.

12. The compound of claim 11 wherein R⁷ and R⁸ each represent methyl.

13. The compound of claim 1, wherein:
Y represents a single bond;
R¹ represents aryl or heteroaryl; and
R² represents C(═O)R^{6b} or C(R⁷)(R⁸)—NR¹⁰R¹¹.

14. The compound of claim 13, wherein R^{6b} represents C₁₋₆alkyl or cycloC₃₋₁₂alkyl.

15. The compound of claim 13, wherein R⁷ and R⁸, which may be the same or different, each independently represent hydrogen or C₁₋₆alkyl and R¹⁰ and R¹¹, together with the nitrogen atom to which they are attached form a monocyclic ring, wherein the ring is optionally substituted by one or more substituents, which may be the same or different, selected independently from C₁₋₆alkyl, C₁₋₆alkoxy, and halogen.

16. The compound of claim 15, wherein R⁷ and R⁸, which may be the same or different, each independently represent hydrogen or methyl and R¹⁰ and R¹¹, together with the nitrogen atom to which they are attached form a piperidine ring, wherein the piperidine ring is optionally substituted by one or more substituents, which may be the same or different, selected independently from C₁₋₆alkyl, C₁₋₆alkoxy, and halogen.

17. The compound of claim 1, which is selected from:
6-Adamantan-1-yl-3-(2,5-dimethoxyphenyl)-imidazo[2,1-b]thiazole,
6-Adamantan-1-yl-3-(2,5-difluorophenyl)-imidazo[2,1-b]thiazole,
6-Adamantan-1-yl-3-(4-methylphenyl)-imidazo[2,1-b]thiazole,
6-Adamantan-1-yl-3-(2,4-dimethylphenyl)-imidazo[2,1-b]thiazole,
6-Adamantan-1-yl-3-(2,4-dimethoxyphenyl)-imidazo[2,1-b]thiazole,
6-Adamantan-1-yl-3-benzo[1,3]dioxol-5-yl-imidazo[2,1-b]thiazole,
6-Adamantan-1-yl-3-(3,4-dimethoxyphenyl)-imidazo[2,1-b]thiazole,
6-Adamantan-1-yl-3-benzofuran-2-yl-imidazo[2,1-b]thiazole,
6-Adamantan-1-yl-3-(4-fluorophenyl)-imidazo[2,1-b]thiazole,
6-Adamantan-1-yl-3-thiophen-2-yl-imidazo[2,1-b]thiazole,
6-Adamantan-1-yl-3-(4-methoxy-3-methyl-phenyl)-imidazo[2,1-b]thiazole,
6-Adamantan-1-yl-3-(3-trifluoromethoxyphenyl)-imidazo[2,1-b]thiazole,
6-(Adamantan-1-yl)-3-(2,4,6-trimethylphenyl)-imidazo[2,1-b]thiazole,
6-Adamantan-1-yl-3-(2-trifluoromethylphenyl)-imidazo[2,1-b]thiazole,
6-Adamantan-1-yl-3-(2,5-diethylphenyl)-imidazo[2,1-b]thiazole,
6-Cyclohexyl-3-(2,5-difluorophenyl)-imidazo[2,1-b]thiazole,
3-(6-Adamantan-1-yl-imidazo[2,1-b]thiazol-3-yl)-5-methoxy-1,2-dimethyl-1H-indole,
6-Adamantan-1-yl-3-(3-bromophenyl)-imidazo[2,1-b]thiazole,
6-Adamantan-1-yl-3-(3-acetylaminophenyl)-imidazo[2,1-b]thiazole,
6-Adamantan-1-yl-3-(3-aminophenyl)-imidazo[2,1-b]thiazole,
3-(2,5-Dimethoxyphenyl)-6-(1-methyl-1-phenylethyl)-imidazo[2,1-b]thiazole,
3-(2,5-Dimethylphenyl)-6-(1-methyl-1-phenylethyl)-imidazo[2,1-b]thiazole,
3-(2,5-Dimethoxyphenyl)-6-piperidin-1-yl-imidazo[2,1-b]thiazole,
6-Azepan-1-yl-3-(2,5-dimethoxyphenyl)-imidazo[2,1-b]thiazole,
3-(2,4-Dimethoxyphenyl)-6-[1-(3-fluorophenyl)-1-methyl-ethyl]-imidazo[2,1-b]thiazole,
3-(2,4-Dimethoxyphenyl)-6-(1-methyl-1-phenyl-ethyl)-imidazo[2,1-b]thiazole,
6-Adamantan-1-yl-3-(3-dimethylaminophenyl)-imidazo[2,1-b]thiazole,
6-Adamantan-1-yl-3-(1,2,5-trimethyl-1H-pyrrol-3-yl)-imidazo[2,1-b]thiazole,
6-Adamantan-1-yl-3-(1-methyl-1H-pyrrol-2-yl)-imidazo[2,1-b]thiazole,
6-Adamantan-1-yl-3-(3-trifluoromethylphenyl)-imidazo[2,1-b]thiazole,
6-Adamantan-1-yl-3-(2,5-dimethylthiophen-3-yl)-imidazo[2,1-b]thiazole,
6-Cyclohexyl-3-(2,5-dimethoxyphenyl)-imidazo[2,1-b]thiazole,
6-Adamantan-1-yl-3-(2,5-dimethylphenyl)-imidazo[2,1-b]thiazole,
4-(6-Adamantan-1-yl-imidazo[2,1-b]thiazol-3-yl)-benzonitrile,
4-(6-Adamantan-1-yl-imidazo[2,1-b]thiazol-3-yl)benzene-1,3-diol,
[3-(6-Adamantan-1-yl-imidazo[2,1-b]thiazol-3-yl)phenyl]-methanol,
3-(2,5-Dimethoxyphenyl)-6-[1-(4-fluorophenyl)1-methyl-ethyl]-imidazo[2,1-b]thiazole,
6-Adamantan-1-yl-3-(3-methoxyphenyl)-imidazo[2,1-b]thiazole,
3-(6-Adamantan-1-yl-imidazo[2,1-b]thiazol-3-yl)phenol,
Acetic acid 3-(6-adamantan-1-yl-imidazo[2,1-b]thiazol-3-yl)phenylester,
5-(6-Adamantan-1-yl-imidazo[2,1-b]thiazol-3-yl)-2-methoxy-phenylamine,
6-Adamantan-1-yl-3-(4-methoxyphenyl)-imidazo[2,1-b]thiazole,
3-(3-bromophenyl)-6-tert-butyl-imidazo[2,1-b]thiazole,
6-tert-Butyl-3-(2,4-dimethylphenyl)-imidazo[2,1-b]thiazole,
6-tert-Butyl-3-(p-tolyl)-imidazo[2,1-b]thiazole,
6-tert-Butyl-3-(3-methoxyphenyl)-imidazo[2,1-b]thiazole,
6-tert-Butyl-3-(2,5-dimethoxyphenyl)-imidazo[2,1-b]thiazole,
6-Adamantan-1-yl-3-(6-aminopyridin-3-yl)imidazo[2,1-b]thiazole,
6-Adamantan-1-yl-3-pyridin-3-yl-imidazo[2,1-b]thiazole,
6-Adamantan-1-yl-3-(6-methoxy-pyridin-3-yl)-imidazo[2,1-b]thiazole,
6-Adamantan-1-yl-3-(2-fluoro-pyridin-3-yl)-imidazo[2,1-b]thiazole,
6-Adamantan-1-yl-3-pyridin-4-yl-imidazo[2,1-b]thiazole,
6-Adamantan-1-yl-3-(2,4-dimethoxy-pyrimidin-5-yl)-imidazo[2,1-b]thiazole,
8-[3-(3-Methoxyphenyl)-imidazo[2,1-b]thiazol-6-yl]-8-aza-spiro[4.5]decane,
8-[3-(3-Methoxyphenyl)-imidazo[2,1-b]thiazol-6-yl]-1,4-dioxa-8-aza-spiro[4.5]decane,
8-{1-[3-(3-Methoxyphenyl)-imidazo[2,1-b]thiazol-6-yl]-1-methyl-ethyl}-8-aza-spiro[4.5]decane,
8-{1-[3-(3-Methoxy-phenyl)-imidazo[2,1-b]thiazol-6-yl]-1-methyl-ethyl}-1,4-dioxa-8-aza-spiro[4.5]decane,
6-Adamantan-1-yl-3-(3,4-difluorophenyl)-imidazo[2,1-b]thiazole,
3-(6-tert-Butyl-imidazo[2,1-b]thiazol-3-yl)benzoic acid methyl ester,
Acetic acid, 3-(6-Adamantan-1-yl-imidazo[2,1-b]thiazol-3-yl)benzyl ester,
2-(6-Adamantan-1-yl-imidazo[2,1-b]thiazol-3-yl)phenylamine,
N[3-(6-Adamantan-1-yl-imidazo[2,1-b]thiazol-3-yl)-phenyl]-methanesulfonamide,
3-(6-Adamantan-1-yl-imidazo[2,1-b]thiazol-3-yl)-benzonitrile,
Acetic acid, 3-acetoxy-4-(6-Adamantan-1-yl-imidazo[2,1-b]thiazol-3-yl)phenyl ester,
Acetic acid, 2-acetoxy-4-(6-Adamantan-1-yl-imidazo[2,1-b]thiazol-3-yl)phenyl ester,
4-(6-Adamantan-1-yl-imidazo[2,1-b]thiazol-3-yl)benzene-1,2-diol,
Acetic acid, 4-acetoxy-3-(6-Adamantan-1-yl-imidazo[2,1-b]thiazol-3-yl)phenyl ester,
2-(6-Adamantan-1-yl-imidazo[2,1-b]thiazol-3-yl)benzene-1,4-diol,
6-Adamantan-1-yl-3-pyridin-2-yl-imidazo[2,1-b]thiazole,
6-Adamantan-1-yl-3-(4-amino-3-methoxyphenyl)imidazo[2,1-b]thiazole,
6-Adamantan-1-yl-3-(3-chlorophenyl)-imidazo[2,1-b]thiazole,
3-(6-Adamantan-1-yl-imidazo[2,1-b]thiazol-3-yl)benzoic acid methyl ester,
6-Adamantan-1-yl-3-(3,5-dimethoxyphenyl)-imidazo[2,1-b]thiazole,
N-[5-(6-Adamantan-1-yl-imidazo[2,1-b]thiazol-3-yl)-2-methoxy-phenyl]-acetamide,
6-Adamantan-1-yl-2-phenyl-imidazo[2,1-b]thiazole,
and optical isomers, polymorphs and pharmaceutically acceptable acid and base addition salts, hydrates, and solvates thereof.

18. A pharmaceutical composition comprising as active ingredient at least one compound as claimed in claim 1 together with one or more pharmaceutically acceptable excipients or vehicles.

19. A method for treating or preventing a condition or disease associated with abnormal glutamate neurotransmission or a method for modulating Group I mGluR receptors to achieve therapeutic benefit, or a method for enhancing cognition, such method comprising the step of administering to a living animal, including a human, a therapeutically effective amount of a compound as claimed in claim 1.

20. The method as claimed in claim 19, wherein the condition associated with abnormal glutamate neurotransmission, or wherein modulation of mGluR receptors results in therapeutic benefit, is selected from: AIDS-related dementia, Alzheimer's disease, Creutzfeld-Jakob's syndrome, bovine spongiform encephalopathy (BSE) or other prion related infections, diseases involving mitochondrial dysfunction, diseases involving β-amyloid and/or tauopathy such as Down's syndrome, hepatic encephalopathy, Huntington's disease, motor neuron diseases such as amyotrophic lateral sclerosis (ALS), multiple sclerosis (MS), olivoponto-cerebellar atrophy, post-operative cognitive deficit (POCD), lupus disease, neuronal ceroid lipofuscinosis, neurodegenerative cerebellar ataxias, Parkinson's disease, Parkinson's dementia, mild cognitive impairment, dementia pugilistica, vascular and frontal lobe dementia, cognitive impairment, eye injuries, eye diseases, eye disorders, glaucoma, retinopathy, macular degeneration, head and brain and spinal cord injuries, head and brain and spinal cord trauma, hypoglycaemia, hypoxia, perinatal hypoxia, ischaemia, ischaemia resulting from cardiac arrest or stroke or bypass operations or transplants, convulsions, epilepsy, myoclonic epilepsy, epileptic convulsions, temporal lobe epilepsy, glioma and other tumours, cancer, oral cancer, squamous cell carcinoma (SCC), oral squamous cell carcinoma (SSC), neoplasia, hyperplasia, dysplasia, cancer, carcinoma, sarcoma, oral cancer, squamous cell carcinoma (SCC), oral squamous cell carcinoma (SCC), lung cancer, lung adenocarcinoma, breast cancer, prostate cancer, gastric cancer, liver cancer, colon cancer, colorectal carcinoma, brain tumor, tumor of a nerve tissue, malignant glioma, astroglioma, neuroglioma, neuroblastoma, glioblastoma, medulloblastoma, cancer of skin cells, melanoma, malignant melanoma, epithelial neoplasm, lymphoma, myeloma, Hodgkin's disease, Burkett's lymphoma, leukemia, thymoma, inner ear insult, inner ear insult in tinnitus, tinnitus, sound or drug-induced inner ear insult, sound or drug-induced tinnitus, L-dopa-induced and tardive dyskinesias, L-dopa-induced dyskinesia in Parkinson's disease therapy, chorea, athetosis, stereotypy, ballism, Tic disorder, torticollis spasmodicus, blepharospasm, focal and generalized dystonia, nystagmus, hereditary cerebellar taxias, corticobasale degeneration, tremor, essential tremor, abuse, addiction, nicotine addiction, nicotine abuse, alcohol addiction, alcohol abuse, opiate addiction, opiate abuse, cocaine addiction, cocaine abuse, amphetamine addiction, amphetamine abuse, obesity addiction, anxiety and panic disorders, attention deficit hyperactivity disorder (ADHD), attention deficit syndrome (ADS), restless leg syndrome, hyperactivity in children, autism, dementia, dementia in Alzheimer's disease, dementia in Korsakoff syndrome, vascular dementia, dementia in HIV infections, major depressive disorder or depression, depression resulting from Borna virus infection, and bipolar manic-depressive disorder, drug tolerance, drug tolerance to opioids, movement disorders, dystonia, dyskinesia, L-Dopa-induced dyskinesia, tardive dyskinesia, dyskinesia in Huntington's disease, fragile-X syndrome, Huntington's chorea, irritable bowel syndrome (IBS), migraine, multiple sclerosis, muscle spasms, pain, chronic pain and acute pain, inflammatory pain, neuropathic pain, diabetic neuropathic pain (DNP), cancer pain, pain related to rheumatic arthritis, allodynia, hyperalgesia, nociceptive pain, post traumatic stress disorder, schizophrenia, positive or cognitive or negative symptoms of schizophrenia, spasticity, Tourette's syndrome, urinary incontinence, vomiting, pruritic conditions, pruritis, sleep disorders, micturition disorders, neuromuscular disorder in the lower urinary tract, gastroesophageal reflux disease (GERD), lower esophageal sphincter (LES) disease, functional gastrointestinal disorders, dyspepsia, regurgitation, respiratory tract infection, bulimia nervosa, chronic laryngitis, asthma, reflux-related asthma, lung disease, eating disorders, obesity and obesity-related disorders, binge eating disorder, agoraphobia, generalized anxiety disorder, obsessive-compulsive disorder, panic disorder, posttraumatic stress disorder, social phobia, substance-induced anxiety disorder, delusional disorder, schizoaffective disorder, schizophreniform disorder, substance-induced psychotic disorder, delirium, or for cognitive enhancement and/or neuroprotection.

21. A composition comprising a combination of a compound as claimed in claim 1 and an NMDA receptor antagonist.

22. The composition as claimed in claim 21, wherein the NMDA receptor antagonist is selected from memantine and neramexane and pharmaceutically acceptable salts, polymorphs, hydrates, and solvates thereof.

23. A method of providing neuroprotection to a living animal, including a human, comprising the step of administering to a living animal, including a human, a therapeutically effective amount of a composition of claim 22.
